SEQUENCE LISTING (8 sequences)

SEQ ID NO: 1 - protein, 15 aa, Artificial Sequence
  protease-sensitive linker
GGGGSGGGGSGGGGS

SEQ ID NO: 2 - protein, 8 aa, Artificial Sequence
  cleavable peptide fragment
LSGRSDNH

SEQ ID NO: 3 - protein, 22 aa, Artificial Sequence
  self-cleaving P2A peptide
GSGATNFSLLKQAGDVEENPGP

SEQ ID NO: 4 - DNA, 19 bp, Artificial Sequence
  primer
acatcgctcagacaccatg

SEQ ID NO: 5 - DNA, 22 bp, Artificial Sequence
  primer
tgtagttgaggtcaatgaaggg

SEQ ID NO: 6 - protein, 451 aa, Artificial Sequence
  hCD3zeta-hNKG2D-P2A-hDAP10/Flag
MRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGSGATNFSLLKQAGDVEENPGPMIHLGHILFLLLLPVAAADYKDDDDKQTTPGERSSLPAFYPGTSGSCSGCGSLSLPLLAGLVAADAVASLLIVGAVFLCARPRRSPAQDGKVYINMPGRG

SEQ ID NO: 7 - protein, 469 aa, Artificial Sequence
  anti-GD2 scFv-CD8H-hCD28-hCD3zeta
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSMRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 1212 aa, Artificial Sequence
  anti-CD73 scFv-linker-cleavable peptide-linker- GD2.CAR-NKG2D.CAR
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSSGGGGSGGGGSGGGGSQSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVLGGGGSGGGGSGGGGSLSGRSDNHGSSGTEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSMRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGSGATNFSLLKQAGDVEENPGPMIHLGHILFLLLLPVAAADYKDDDDKQTTPGERSSLPAFYPGTSGSCSGCGSLSLPLLAGLVAADAVASLLIVGAVFLCARPRRSPAQDGKVYINMPGRG

(19) United States
(12) Patent Application Publication
(10) Pub. No.: US 2022/0362299 A1
(43) Pub. Date: Nov. 17, 2022

(54) ENGINEERED NATURAL KILLER CELLS AND METHODS FOR USING THE SAME IN IMMUNOTHERAPY AND AUTOPHAGY INHIBITON TECHNIQUES

(71) Applicant: Purdue Research Foundation, West Lafayette, IN (US)

(72) Inventors: Sandro Matosevic, Lafayette, IN (US); Jiao Wang, West Lafayette, IN (US)

(21) Appl. No.: 17/770,585

(22) PCT Filed: Oct. 21, 2020
(86) PCT No.: PCT/US2020/056723

§ 371 Date: Apr. 20, 2022

Related U.S. Application Data
(60) Provisional application No. 62/923,644, filed on Oct. 21, 2019.

(51) Int. Cl. A61K 35/17; C07K 14/705; A61P 35/00

(52) U.S. Cl. CPC A61K 35/17; C07K 14/70596; C07K 14/705; A61P 35/00

(57) ABSTRACT

Polynucleotide constructs and multifunctional engineered natural killer (NK) cells expressing such constructs are provided for the treatment of cancer and, in particular, glioblastoma. The constructs are a fusion of a first binding domain that targets at least one cognate ligand on a target cell, a second binding domain specific for an adenosine producing cell surface protein of the target cell or an adenosine-intermediary producing cell surface protein of the target cell and a cleavable linker, and a third binding domain specific for a cancer-associated antigen. Pharmaceutical compositions of the engineered NK cells are also provided, as well as methods of treating glioblastoma using such pharmaceutical compositions alone and in addition to autophagy inhibitors.

### PRIORITY

This application is related to and claims priority benefit of U.S. Provisional Patent Application Ser. No. 62/923,644 to Matosevic et al. filed Oct. 21, 2019. The content of the aforementioned application is hereby incorporated by reference in its entirety into this disclosure.

### FIELD

This disclosure relates in general to the field of cancer therapies and treatments and, more particularly, to multifunctional immunotherapies that utilize natural killer (NK) cells engineered to bear multiple anti-tumor functions and address key drivers of cancer resistance, and in particular glioblastoma resistance, to therapies.

### BACKGROUND

Glioblastoma (GBM) is by far the most common and aggressive malignant type of primary brain tumor in adults and children and one of the most difficult to treat. GBM patients tend to be poorly responsive to traditional treatments and GBM has the worst prognosis of any central nervous system malignancy. Even with advancing diagnostic modalities and aggressive single and multimodal treatment options such as surgery, chemotherapy, radiation techniques, small molecule inhibitors, and the use of multiple antineoplastic drugs, survival rates have only modestly improved over the past several decades, with a median survival of approximately one year. Indeed, of the conventional treatments tested to date, all have failed to improve GBM patient overall survival in phase III clinical trials and a cure for GBM does not exist. Reasons for this failure are multifactorial.

Primarily, GBM is highly infiltrative and invasive by nature, and therapy for GBM is difficult due to its biological location in the brain, the blood brain barrier, and neural parenchyma. Further, the heterogeneity of GBM, along with the complex interactions among different cells within as well as cells surround the tumor, have been appointed as one of the main causes of therapeutic resistance and malignant relapse. Each of the known GBM subtypes (classical, mesenchymal, neural and proneural) display diverse genetic and epigenetic signatures associated with distinct and variable cell plasticities.

A subpopulation of GMB cells, glioma stem-like cells (GSCs) also contribute to treatment resistance. GSCs are capable of demonstrating self-renewal capacity, multi-potency, and induction of tumorigenesis, and are increasingly being recognized as a driving force supporting glioma genesis, resistance to therapy and aggressive recurrence. This is at least in part due to the failure of conventional therapies to eliminate specific GSC subpopulations. However, these cells are poorly recapitulated by conventional GBM model cell lines (including U87MG) which hinders the study of GMB.

GBM progression is also promoted by autophagy—a highly controlled catabolic regulator of cellular energetic balance. Under normal conditions, cells utilize basal levels of autophagy to aid in the maintenance of biological function, homeostasis, quality-control of cell contents, and elimination of old proteins and damaged organelles. In cancer cells, however, authophagy can facilitate tumorigenesis by promoting cancer-cell proliferation and tumor growth. Indeed, autophagy can support GBM metabolism, survival in hypoxia, progression and resistance to therapy. Beclin-1, encoded by the BECN1 gene, has a central role in the promotion of autophagy.

Additionally, GBM expresses multiple immune checkpoints which can either participate in antigen evasion or drive immunosuppression. One of these is ecto-5′-nucleotidase (CD73), a surface enzyme expressed on multiple cells (including both infiltrating immune cells and tumor cells) that mediates the gradual hydrolysis of ATP and ADP to anti-inflammatory adenosine and is upregulated in GBM. CD73, in association with CD39, induces the production of extracellular adenosine from adenosine 5′-triphosphate (ATP). Adenosine, in turn, binds to adenosine receptors on natural killer (NK) cells, resulting in significant immunometabolic dysregulation of NK cell activity. In this manner, immune suppression mediated by adenosinergic pathways, which is very important for maintaining immune system homeostasis, is hijacked in GBM.

In addition to the foregoing, treatment evasion by GBM is also fueled by a heavily immunosuppressive, hypoxic tumor microenvironment (TME), which sets off both metabolic and functional cascades that suppress important effectors of the body's innate immune system. The pathophysiologic conditions of hypoxia and ischemia, such as those found in tumors' TMEs due to inadequate vascularization, also drive the significant metabolic changes in adenine nucleotides such ATP and adenosine diphosphate (ADP). Under normal physiological conditions, ATP is localized in the intracellular compartment; however, levels of extracellular ATP (and thus adenosine) rise significantly in response to hypoxia, ischemia and the setting of malignancy, defining features of the tumor environment. For example, intratumoral extracellular ATP concentrations can be up to 1,000 times higher than those in normal tissues of the same origin cell. These conditions contribute to the dysregulation of NK cells and, thus, suppression of NK cell anti-tumor surveillance and immunity which fuels the tumor's invasiveness.

As important effectors of innate immunity, NK cells are unique and play pivotal functions in cancer immunity surveillance. Unlike T cells that only detect major histocompatibility complex (MHC) presented on infected cell surfaces, NK cell function is driven by a balance of activating and inhibitory receptors through which they interact with pathogens and recognize MHC class I molecules on cancer cells. NK cells can eliminate a variety of abnormal or stressed cells without prior sensitization and even preferentially kill stem-like cells or cancer stem cells. Upon forming immune synapses with target cells, NK cells release cytokines that induce cell lysis. However, GBM employs various tactics to delay, alter, or even stop immune suppressive pathways to prevent the malignant cells from being recognized as dangerous or foreign. These mechanisms prevent the cancer from being eliminated by the immune system, leading to failures in the control of tumor growth and allowing for disease to progress from a very early stage to a lethal state.

Alongside GBM-induced functional inhibition of NK cell responses, the downregulation or mutation of target antigens is commonly observed in GBM and also contributes to immune evasion and resistance to treatment. Though conventional adoptive transfer T cell therapy strategies including dual antigen-targeting or programmable, tumor-sensing chimeric antigen receptors (CARs) have been evaluated pre-clinically to combat such evasion, GBM employs mechanisms beyond antigen escape to avoid targeting. Antigen escape results in decreased efficacy of cell-based antigen-specific monotherapy and is triggered by mechanisms including differential splicing, missense mutations, or lineage switch. Indeed, outgrowth of antigen escape variants has been observed in all clinical studies to date with most GBM-specific and GBM-associated antigens.

There is a dire need for new and effective GBM treatment options. While recapitulating NK cell function lost to multiple complex mechanisms not only presents a significant challenge to conventional therapies, it also requires a larger presence of NK cells specifically within the tumor tissue so that a meaningful clinical response can be mounted. Conventional methods have not been successful in these areas. Therefore, a need exists to develop a commercially viable and safe method for effective GBM treatment options capable of enhanced GBM tumor targeting, increased NK cell recruitment to the TME, and through which NK dysfunction can be rescued.

### SUMMARY

The present disclosure describes the development of novel immunotherapies that target multiple immune evasion mechanisms in GBM. The compositions, systems, and methods hereof combine, for the first time, genetically-engineered natural killer (NK) cells designed to target two or more GBM antigens at once, while also releasing an antibody to block CD73 activity. In at least one embodiment, the present disclosure further combines this cell-based immunotherapy with a small molecule autography inhibitor, for example and without limitation chloroquine. The use of an autography inhibitor in this context results in further GBM inhibition, as well as the secretion of chemokines that attract NK cells to further infiltrate GBM and enhance overall therapy effectiveness. In this manner, the compositions, systems and methods of the present disclosure can restrict GBM escape from immune surveillance and promote recruitment of NK cells to the tumor, thus resulting in sustained anti-GBM responses.

Polynucleotide constructs are provided herein. In at least one embodiment, such constructs comprise: a first sequence encoding at least a first binding domain or fragment thereof operatively linked to a second sequence encoding at least a second binding domain or fragment thereof. The first binding domain or fragment thereof comprises a NK activating receptor or a first protein specific for a first cancer-associated antigen, and the second binding domain or fragment thereof is specific for an adenosine producing cell surface protein of the target cell or an adenosine-intermediary producing cell surface protein of the target cell. The first domain may optionally further encode a hinge domain (e.g., and without limitation, a linker or a spacer), one or more self-cleaving peptides (e.g., and without limitation, P2A, E2A, F2A, T2A), or both. The second binding domain/fragment further comprises a cleavable linker operably linked to the first binding domain. In certain embodiments, the cleavable linker is configured to be cleavable by one or more proteases present in the target cell and/or tumor microenvironment (TME) (such proteases are widely known and understood in the art including, without limitation, lysosomal cysteine proteases, serine protease (such as trypsin), aspartate proteases, threonine proteases, and matrix metalloproteases). In at least one exemplary embodiment, the second binding domain comprises an antibody fragment specific for CD73, CD39, or CD38 and, optionally, may comprise a single chain antibody fragment (scFv).

Exemplary embodiments of the construct further comprise a third sequence encoding at least a third binding domain or fragment thereof. Such third binding domain/fragment comprises a NK activating receptor or a second protein specific for a second cancer-associated antigen. For example, the first binding domain or fragment thereof may comprise a first protein specific for a cancer-associated antigen, and the third binding domain or fragment thereof may comprise an NK activating receptor (e.g., and without limitation, natural killer group 2 member D receptor (NKG2D), NKp30, NKp46, NKp40, or DNAM-1). In at least one exemplary embodiment, the first sequence encodes a first amino acid that is at least 90% identical to SEQ ID NO: 7 and the third sequence encodes a second amino acid that is at least 90% identical to SEQ ID NO: 6.

The cancer-associated antigens of the present disclosure (to which one or more of the binding domains or fragments may be specific) may include one or more of, without limitation, isialoganglioside (GD2), ganglioside G3 (GD3), Her 2 (p185), CD19, CD20, CD56, CD123, CD22, CD30, CD33, CD171, CS-1, C-type lectin-like molecule-1; EpCAM, G250, proteoglycans, GD3, GD2, MHC II, TAG-72, milk mucin core protein, Lewis A antigen, tyrosine-protein kinase transmembrane receptor (ROR1), c-met, epidermal growth factor receptor (EGFR), EGFR variant III, and/or carcinoembryonic antigen (CEA). Furthermore, the first cancer-associated antigen may comprise one type of antigen, whereas the second cancer-associated antigen may comprise the same or a different type of antigen, as desired.

In at least one exemplary embodiment, the first and/or third binding domain or fragment thereof comprises an extracellular ligand-binding domain comprising a NKG2D and the respective sequence further encodes upregulated expression of the NKG2D as compared to expression of NKG2D in a wild-type NK cell. Additionally or alternatively, the second binding domain or fragment thereof may comprise an anti-CD73, an anti-CD39, or an anti-CD38 linked with a scFv, and the first and/or third binding domain or fragment thereof may comprise an anti-GD2 linked with a scFv.

Still further, the first sequence may be expressed in a first chimeric antigen receptor (CAR) and the second sequence is expressed in a second CAR. Further, the target cell may be a cancer cell, a malignant cell in a TME and, in at least one exemplary embodiment, the target cell is a glioblastoma cell or a glioblastoma TME.

In at least one exemplary embodiment, one or both of the first and third sequences may additionally encode one or more signaling domains for promoting cytotoxic or cytolytic activity upon activation. There, such signaling domains are activated upon the binding domain or fragment thereof operably linked thereto binding the target cell. The one or more signaling domains may be selected from a group consisting of an immunoglobulin γ-Fc region receptor III-A (FcγRIIIA), a cluster of differentiation 28 (CD28), a tumor necrosis factor receptor superfamily member 9 (TNFRSF9 or 4-1BB), a tumor necrosis factor receptor superfamily member 4 (TNFRSF4 or OX40), a Fas ligand (FasL), a TNF-related apoptosis-inducing ligand (TRAIL), DNAX-activating protein 10 (DAP10), DNAX-activating protein 12 (DAP12), natural cytotoxicity receptor NKp46, natural cytotoxicity receptor NKp44, natural cytotoxicity receptor NKp30, lymphocyte function-associated antigen 1 (LFA-1), cluster of differentiation 244 (CD244), CD137, CD3 zeta (CD3ζ) and a NKG2D-DAP10 receptor complex. In at least one exemplary embodiment, one or more of the signaling domains encoded by the first sequence is a DAP10 and one or more of the signaling domains encoded by the second sequence is a CD3ζ.

Engineered cells or engineered cell lines are also provided herein. In at least one embodiment, an engineered cell or engineered cell line is provided that expresses any one of the polynucleotide constructs disclosed herein. Further, the engineered cell or engineered cell line may comprise the first sequence that further encodes a hinge domain operably linked to and positioned between the first binding domain or fragment thereof and a signaling domain encoded thereby. In at least one exemplary embodiment, the engineered cell or cell line expresses an amino acid sequence that is at least 80%, 85% or 90% identical to SEQ ID NO: 8.

Other embodiments provide for pharmaceutical compositions comprising a population of the engineered cells of the present disclosure. In such embodiments, one or more signaling domains may be activated upon the binding domain or fragment thereof operably linked thereto binding the target cell, such as for example and without limitation, a cancer or glioblastoma tumor cell. Here, the pharmaceutical composition may comprise a pharmaceutically acceptable carrier and/or any pharmaceutically acceptable diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents, and dispensing agents (depending on the nature of the mode of administration and dosage forms).

Methods are also provided for treating a subject suffering from a cancer using an immunotherapy treatment. In at least one embodiment, such a method comprises administering, or having administered, to a subject a therapeutically effective amount of a pharmaceutical composition comprising a population of engineered NK cells expressing a polynucleotide construct encoding at least: a first binding domain or fragment thereof comprising an NK activating receptor or a first protein specific for a first cancer-associated antigen, and a second binding domain or fragment thereof and a cleavable linker, the second binding domain specific for an adenosine-producing or adenosine-intermediary-producing cell surface protein of a target cell and the cleavable linker operably linked to the first binding domain. In certain embodiments, the polynucleotide construct may further encode a third binding domain or fragment thereof comprising a NK activating receptor or a second protein specific for a second cancer-associated antigen. There, the first binding domain or fragment thereof may comprise a first protein specific for a cancer-associated antigen and the third binding domain or fragment thereof comprises an NK activating receptor and the third binding domain or fragment thereof (or vice versa). The polynucleotide construct may additionally encode one or more signaling domains operably linked to the first and/or third binding domain(s)/fragment(s) to promote cytotoxic or cytolytic activity of the engineered NK cells upon activation.

The step of administering (or having administered) a therapeutically effective amount of the pharmaceutical composition may be performed intravenously, intratumorally, parenterally, or via infusion. Additionally or alternatively, the step of administering, or having administered, to a subject a therapeutically effective amount of a pharmaceutical composition may comprise performing, or having performed, adoptive cell therapy.

Such methods may employ a population of engineered NK cells express an amino acid sequence that is at least 80%, 85%, or 90% identical to SEQ ID NO. 8.

In at least one embodiment, the cancer may be glioblastoma. Additionally or alternatively, the first binding domain or fragment thereof may comprise a first protein specific for GD2, the third binding domain or fragment thereof may comprise an extracellular ligand-binding domain comprising a NKG2D, and the third binding domain or fragment thereof may be specific for CD73, CD39, or CD38.

Other methods of a combination treatment are also provided. In at least one embodiment, the method may further comprise administering, or having administered, to the subject an additional therapeutic treatment comprising an autophagy inhibitor. Such autophagy inhibitor may be, for example and without limitation, a therapeutically effective amount of a small molecule inhibitor and/or the genetic downregulation of a gene in the autophagy pathway (such as, for example, BECN1, p62, β-actin, ATG5, ATG7, LC3B, ATG12, ATG16L1 PI3K-III, ULK1, ULK2, FIP200, and/or LAMP2). Non-limiting examples of such small molecule inhibitors may comprise chloroquine, hydroxychloroquine, spautin-1, SAR405, vertoprofin, and any other pharmaceutical autophagy inhibitor or down-regulator now known or hereinafter discovered. In at least one exemplary embodiment, the autophagy inhibitor comprises chloroquine which, optionally, may be administered at a concentration of between 0.01 μM and 200 μM.

Where the methods hereof comprise combination treatments, the steps of administering (or having administered) to a subject a therapeutically effective amount of a pharmaceutical composition may be performed intravenously, intratumorally, parenterally, or via infusion, and the step of administering, or having administered, to the subject an additional therapeutic treatment may be performed via systemic injection or infusion.

Kits for treating a subject experiencing glioblastoma are also provided. For example, and without limitation, such kits may comprise a therapeutically effective amount of the pharmaceutical composition of the present disclosure; and a therapeutically effective amount of an autophagy inhibitor (whether a pharmaceutical inhibitor, such as a small molecule inhibitor or otherwise) or a composition to achieve the genetic downregulation of the autophagy pathway).

Where the autophagy inhibitor in the kit comprises a small molecule inhibitor, in at least one embodiment the inhibitor may be selected from a group consisting of chloroquine, hydroxychloroquine, spautin-1, SAR405, and vertoprofin.

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1 results from the correlative analysis of gene expression in glioblastoma (GBM) patient data, surface expression of CD73, GD2, and NKG2DL in patient-derived GBM, and illustrate the design of the multifunctional NK-based GBM immunotherapy, with subpart A showing the correlation between normalized expression (FPKM) of selected genes using data from 156 GBM patients (Pearson's correlation coefficients are shown with continuous gradient colors); subpart B showing the correlation between normalized expression (FPKM) of the entire NK gene set and individual genes (correlation expressed as normalized enrichment scores (NES); subpart C showing a Venn diagram representing the number of GBM patients with high expression of at least one of the identified 4 genes: NT5E, B4GALNT1, MICA and MICB; subpart D showing a bar graph representing the patient distribution of gene expression in GBM tumors based on the four genes in identified in subpart C; subpart E showing data regarding surface expression of CD73, GD2 and NKG2DL on different types of patient-derived GBM cells including SJ-GBM2 (pediatric), GBM43 (primary) and GBM10 (recurrent) determined by flow cytometry (with results reported as fold-change (FC) over control; data shown as mean±SEM; and subpart F showing a schematic illustration representative of at least one embodiment of the multifunctional, tumor-responsive engineered NK cells of the present disclosure and their working mechanisms (where TAP stands for tumor associated protease; αCD73 scFv stands for anti-CD73 scFv; and αGD2 scFv stands for anti-GD2 scFv);

FIG. 2 shows graphical data relating to the effects of adenosine on pNK cell viability and activating marker expression, with subpart A showing graphical data related to cell viability (%) of pNK cells after treatment with 1000 μM of adenosine for 24 h; and subpart B showing graphical data related to NKG2D expression on pNK cells after treatment with 1000 μM of adenosine for 24 h (data are shown as mean±SEM);

FIG. 3 shows in silico modeling of the structure of certain constructs of the present disclosure, with subpart A showing a molecular model of tandem anti-GD2 and anti-CD73 scFv extracellular domains linked together via a GS spacer and a cleavable peptide linker (identified by the arrow), the model obtained based on vector sequences using RaptorX, and subpart B showing the docking of extracellular scFv domains with GD2 (indicated by the dashed circle), with docking performed using PatchDock and refined in FireDock using a model of the entire extracellular region shown in subpart A and the cleavable peptide indicated by the arrow (images generated in Chimera);

FIG. 4 shows a schematic representation of at least one embodiment of a multifunctional construct according to at least one embodiment of the present disclosure;

FIG. 5 shows graphical data related to the generation of multifunctional genetically-engineered NK cells of the present disclosure, with subpart A shows a schematic representation of a transgene for a NKG2D.DAP10.CD3ζ-CAR construct (Construct 1B) pursuant to the present disclosure that targets NKG2D ligands (NKG2DL), and subpart B showing a schematic representation of the structure of Construct 1B; subpart C shows graphical data related to cell viability (%) of NK cells engineered to express Construct 1B pursuant to the present disclosure (NKG2D.CAR-NK92), or non-engineered controls (NK92), 48 hours after transfection; subpart D shows NKG2D expression determined by flow cytometry on transfected cells of subpart C; subpart E shows data related to the in vitro cytotoxicity of the cells of subpart C (NKG2D.CAR-NK92) and non-transfected NK-92 cells (NK92) against GBM43 cells at an E/T ratio of 5 over 4 h; subpart F shows a schematic representation of a transgene structure representing the anti-GD2.CD28.CD3ζ-CAR construct (Construct 1A) pursuant to the present disclosure that targets GD2 and subpart G showing a schematic representation of the structure of Construct 1A (anti-GD2.CD28.CD3ζ-CAR); subpart H shows the cell viability (%) of NK-92 cells engineered to express Construct 1A (GD2.CD28.CD3ζ-CAR), or non-engineered controls (NK92) after 48 h transfection; subpart I shows anti-GD2 scFv expression determined by flow cytometry on transfected cells of subpart H; subpart J showing data related to the in vitro cytotoxicity of the cells of subpart H (anti-GD2.CD28.CD3ζ-CAR-NK92) and non-engineered NK-92 controls against GBM43 cells at an E/T ratio of 5 over 4 h; subpart K shows a schematic representation of transgene representing the complete multi-functional Construct 1 according to the present disclosure, Construct 1 comprising two CARs separated by a P2A, the first CAR targeting GD2 with CD28 and CD3ζ signaling domains and linked by a tumor-associated protease cleavable linker to anti-CD73 scFv, which inhibits or otherwise blocks the production of adenosine from GBM once released locally, and the second CAR construct comprising NKG2D-DAP10-CD3ζ and targeting NKG2D ligands an GBM tumor-responsive; subpart L shows a schematic representation of tumor-responsive anti-CD73 scFv secreting dual-specific CAR (Construct 1); subpart M shows data related to the cell viability (%) of NK-92 cells engineered to express the full multi-functional Construct 1 after 48 h transfection; subpart N shows NKG2D expression determined by flow cytometry after 48 h transfection; subpart O shows expression of anti-CD73 scFv and anti-GD2 scFv on NK cells determined by flow cytometry following proteolytic cleavage with uPA to release the anti-CD73 ScFv fragment; subpart P comprises flow cytometry data showing the purity of isolated peripheral blood-derived NK (pNK) cells (CD56⁺CD3⁻); subpart Q shows data related to the cell viability (%) of pNK cells engineered to express the full construct after two rounds of lentiviral transduction; subpart R shows NKG2D expression on engineered pNK cells determined by flow cytometry after two rounds of lentiviral transduction; subpart S shows graphical data related to the expression of anti-CD73 scFv and anti-GD2 scFv on pNK cells determined by flow cytometry after two rounds of lentiviral transduction; and subpart T depicts data representative of the fold expansion of non-transduced pNK and CD73.mCAR-NK cells in NK MACS® medium; (the data shown through subparts P-T is for isolated pNK cells in triplicates from a representative donor; data in FIG. 5 shown as mean±SEM. *p<0.05, **p<0.0);

FIG. 6 shows graphical data related to studies on primary NK cells, with subparts A-D and E-H showing the data for the pNK cells isolated from two separate donors, respectively, where the characterization and expression measurements investigated as described in connection with Example 1 (data shown as mean±SEM. *p<0.05, **p<0.0);

FIG. 7 shows graphical data related to in vitro effector activity of multifunctional genetically-engineered NK cells according to at least one embodiment of the present disclosure against patient-derived GBM, where subpart A shows the in vitro cytotoxicity of NK-92 (control) and CD73.mCAR-NK92 cells (expressing Construct 1) against GBM43 cells at indicated E/T ratios over 4 h; subpart B shows bar graphs representative of the degranulation of NK-92 and CD73.mCAR-NK92 cells [% CD107 and (MFI) CD107] after 4 h coculture with GBM43 cells (E/T ratio, 5:1) (NK cells were analyzed by flow cytometry for surface CD107a expression as a marker of degranulation); subpart C shows IFN-γ production by NK-92 and CD73.mCAR-NK92 cells (% IFN-γ) after 4 h coculture with GBM43 cells (E/T ratio, 5:1); subpart D shows in vitro cytotoxicity of CD73.mCAR-NK92 and CD73.mCAR-NK92 (following aCD73 scFv cleavage) cells against GBM43 cells at indicated E/T ratios over 4 h; subpart E shows CD73 activity of GBM43 cells after incubation with cleaved aCD73 scFv following release from uPA-treated CD73.mCAR-NK92 cells; subparts F-H show in vitro cytotoxicity of pNK and CD73.mCAR-pNK cells against different GBM cells, including SJ-GBM2, GBM43 and GBM10 cells, at indicated E/T ratios over 4 h; subpart I shows degranulation of pNK and CD73.mCAR-pNK cells (% CD107) after 4 h coculture with GBM43 cells (E/T ratio, 5:1) (NK cells were analyzed by flow cytometry for surface CD107a expression as a marker of degranulation); subpart J shows IFN-γ production of pNK and CD73.mCAR-pNK cells (% IFN-γ) after 4 h coculture with GBM43 cells (E/T ratio, 5:1); subpart K shows in vitro cytotoxicity of pNK and CD73.mCAR-pNK (following aCD73 scFv cleavage) cells against GBM43 cells at indicated E/T ratios over 4 h; subpart L shows CD73 activity of GBM43 cells after incubation with cleaved aCD73 scFv following cleavage from uPA-treated CD73.mCAR-NK cells; subpart M shows the relative decrease in CD16 expression on pNK and CD73.mCAR-pNK cells (% of MFI) after 12 h coculture with GBM43 cells (E/T ratio, 5:1); subpart N shows the relative increase in NKG2A expression on pNK and CD73.mCAR-pNK cells (% of MFI) after 12 h coculture with GBM43 cells (E/T ratio, 5:1); and subparts O and P show the in vitro cytotoxicity of pNK and CD73.mCAR-pNK cells against nonmalignant neural cell lines hCMEC/D3 and HCN-2 at indicated E/T ratios over 4 h (data shown through subparts F-P is for isolated pNK cells in at least triplicates from a representative donor; data shown as mean±SEM; *p<0.05, **p<0.01);

FIG. 8 shows a schematic diagram of the reaction employed to detect phosphate formation by CD73 (subpart A), and a phosphate standard curve in a 96-well plate, where the OD value at 620 nm was read after a 30 min incubation (subpart B);

FIG. 9 shows live imaging micrograph images (taken using an IncuCyte S3) of co-culture of GBM43 cells with pNK or CD73.mCAR-pNK cells (pNK cells expressing Construct 1 of the present disclosure), with the small, irregularly-shaped circular dots in the images representing NK cells and the large fusiform cells representing GBM43 cells (scale bar=200 μm);

FIG. 10 shows data relating to the surface expression of CD73, GD2, and NKG2DL on hCMEC/D3 and HCN-2 cells, with expression levels of the three markers on normal brain cell lines determined by flow cytometry and reported as fold-change (FC) overcontrol; data shown as mean±SEM;

FIG. 11 shows graphical data related to the inhibition of autophagy in GBM cells either through BECN1 gene knockdown or pharmacological treatment with CQ, where subpart A shows data from GBM43 cells transfected with BECN1 shRNA (h) lentiviral particles or treated with 50 μM of CQ for 24 h and, thereafter, the cells were lysed and expression levels of BECN1, LC3B, and p62 were analyzed via flow cytometry (with β-actin used as the loading control), and subpart B shows the quantification of relative expression levels of BECN1 (BECN1/β-actin), LC3B (LC3B/β-actin), and p62 (p62/β-actin) (noting that Beclin 1 (BECN1), LC3-II (LC3B) and p62 are the three primary markers of autophagy);

FIG. 12 shows data relating to the effects of targeting autophagy in GBM on NK cell function and homing, where subpart A represents the viability of GBM43 cells after treatment with various concentrations of CQ for 24 h in vitro; subpart B shows cell viabilities (%) plotted versus the Log [CQ concentration (μM)] with the IC₅₀ computed using Prism 5 (GraphPad Software Inc., California); subparts C and D show expression of CCL5 and CXCL10 mRNA in GBM43 cells transfected with BECN1 shRNA lentivirus (BECN1⁻ GBM43) or in cells treated with CQ (results reported as a fold-change (FC) compared to non-transfected controls; subparts E and F show quantification by ELISA of CCL5 and CXCL10 in the supernatant of GBM43 cells following treatment with CQ for 24 h in the presence or absence of various inhibitors, including LY294002, BAY11-7082 and SP600125; subpart G shows a schematic of in vitro experimental bilayer BBB model representing the experimental setup used to evaluate the effects of CCL5 or CXCL10 on pNK cell migration across the BBB; subpartH shows the transmigration of pNK cells through experimental BBB; subpart I shows data regarding the in vitro viability of GBM43 and BECN1⁻ GBM43 cells as determined by CCK-8 assay; subpart J shows a schematic of the experimental design to evaluate the effects of targeting autophagy through BECN1 knockdown on tumor growth and NK cell infiltration (i.e. GBM43 cells were injected into right flank and BECN1⁻ GBM43 cells were injected into the left flank of Rag1^{-/-} mice); subpart K shows data from tumor growth being monitored and recorded on the indicated days; subpart L shows images of immunohistochemical (IHC) staining of NK cells (top row), CCL5 (middle row) or CXCL10 (bottom row) performed on indicated tumor sections using anti-NKp46, anti-CCL5 and anti-CXCL10 antibodies, respectively (Bar=50 μm; 200× magnification); subpart M shows a quantification of NK cells infiltrating control and BECN1⁻ tumors, with cell counts were recorded in 4 consecutive high-power fields (HPFs) at 200× magnification; subparts N and O graph the IHC scores of CCL5 and CXCL10 in control and BECN1⁻ tumors (Note: the data shown in subpart H is for isolated pNK cells from one representative donor in at least triplicates); data are shown as mean±SEM; *p<0.05, **p<0.01;

FIG. 13 shows graphical data relating to chemokine expression in response to inhibition of autophagy on GBM, where expression of CCL2 and CXCL12 mRNA in BECN1⁻ GBM43 or CQ-treated GBM43 cells determined by RT-PCR, GAPDH used as the reference gene (control) and results reported as a FC over control; data shown as mean±SEM; *p<0.05, **p<0.01;

FIG. 14 relates to the use of ELISA array to test concentrations of different types of chemokines in GBM43 cultured medium, with subpart A showing a schematic diagram of the whole experimental process and subpart B showing the determined concentrations of each tested chemokine;

FIG. 15, subpart A shows immunohistochemical (IHC) staining of mouse (Rag1^{-/-}) NK cells performed on indicated GBM43 (control) subcutaneous xenograft tumor sections, and subpart B shows IHC staining of mouse (Rag1^{-/-}) NK cells performed on indicated BECN1⁻ GBM43 subcutaneous xenograft tumor sections, with the IHC staining indicating an enhanced infiltration of NK cells into both peripheral and internal regions of the tumor tissues of BECN1⁻ GBM43 subcutaneous xenograft tumors;

FIG. 16 is graphical data related to the sensitization of GBM cells to NK cell-mediated killing following targeting of autophagy, where subpart shows NKG2DL expression (MFI and %) on GBM43 cells after 24 h treatment with various concentrations of CQ; subpart B shows CD73 expression (MFI and %) on GBM43 cells after 24 h treatment with various concentrations of CQ; subpart C shows CD73 activity of GBM43 cells after treatment with various concentrations of CQ for 24 h; subpart D shows GD2 expression (MFI and %) on GBM43 cells after 24 h treatment with various concentrations of CQ; subpart E shows killing activity of pNK cells against GBM43 cells after treatment with different concentrations of CQ for 24 h; subpart F shows killing activity of NK92 cells and pNK cells against BECN1⁻ GBM43 cells after 4 h co-incubation at an E/T ratio of 5; all data in FIG. 16 are shown in at least triplicates from one representative donor; data are shown as mean±SEM; *p<0.05, **p<0.01;

FIG. 17 relates to the anti-GBM activity of multifunctional engineered NK cells of the present disclosure in a GMB43 xenograft model, with subpart A showing a schematic diagram that illustrates the in vivo treatment program; subpart B showing graphical data relating to tumor growth of individual treatment groups, including PBS, CQ, pNK cells and CD73.mCAR-pNK cells expressing Construct 1 of the present disclosure (tumor size determined by caliper measurements); subpart C showing graphical data related to average tumor weight of the mice in each treatment group after necropsy on day 28 post-start of treatment; subpart D graphing changes in the body weight of the mice in each group during the treatment period; subpart E showing images of immunofluorescence (IF) staining of NK cells (light spots) and cleaved anti-CD73 scFv (indicated by arrows) performed on indicated tumor sections in each treatment group using anti-NKp46 antibody and Protein L (Bar=50 μm; 200× magnification); subpart F showing IHC staining of CD73 performed on indicated tumor sections using anti-CD73 antibody in different treatment groups (Bar=100 μm; 200× magnification; subpart G graphing IHC scores of CD73 expression performed on indicated tumor sections in different treatment groups; subparts H and I showing IHC staining of CCL5 (subpartH) and CXCL10 (subpart I) performed on indicated tumor sections in different treatment groups using anti-CCL5 and anti-CXCL10 antibodies, respectively (Bar=50 μm; 200× magnification); subpart J showing IHC scores of CCL5 or CXCL10 performed on indicated tumor sections in different treatment groups (noting: the data illustrated in this study are for isolated pNK cells from one representative donor in at least triplicates; data shown as mean±SEM; *p<0.05, **p<0.01;

FIG. 18 shows data relating to the generation of luciferase-expressing GBM43 (GBM43-Luc) cells, where GBM43 cells were transfected with commercial luciferase (Luc) lentiviral particles expressing a firefly luciferase 3 gene under an inducible suCMV promoter, puromycin-resistant cells were collected following transduction, and luciferase expression was detected by bioluminescence measurement using an IVIS spectrum, with subpart A showing a representative bioluminescence image indicating the ability of GBM43-Luc cells to express luciferase (signal intensity as a function of cells seeded is shown); subpart B shows graphical data representative of fluorescence intensity as a function of GBM43-Luc cell number (Total flux (photons/sec) quantified using AURA software); subpart C showing growth curves of the GBM43-Luc and parental control GBM43 cells (Data shown as mean±SEM); and

FIG. 19 shows data relating to the in vivo activity of CD73.mCAR-pNK cells in combination with the inhibition of autophagy in an orthotopic GBM43 xenograft model with subpart A showing a schematic diagram illustrating the in vivo treatment program; subparts B and C showing tumor volumes in individual mice of each group over time monitored using bioluminescent imaging; subpart D showing a line graph depicting average tumor size of the mice in each treatment group after necropsy on day 28 post-start of treatment; subpart E showing a line graph of changes in the body weight of the mice in each group during the treatment period; subpart F showing images of IHC staining of CCL5 (left two panels) and CXCL10 (right two panels) performed on indicated tumor sections in different treatment groups using anti-CCL5 and anti-CXCL10 antibodies, respectively (Bar=50 μm; 200× magnification); subpart G showing images of IHC staining of NK cells performed on indicated tumor sections using anti-NKp46 antibody (Bar=50 μm; 200× magnification); subpart H showing a bar graph depicting the quantification of NK cell infiltration into intracranial tumors treated with CD73.mCAR-pNK or CQ+CD73.mCAR-pNK (here, cell counts were recorded in 4 consecutive high-power fields (HPFs) at 200× magnification; subpart I showing images of IHC staining for CD73 performed on indicated tumor sections using anti-CD73 antibody in different treatment groups (Bar=50 μm; 200× magnification); subpart J showing graphical data depicting IHC scores of CD73 expression performed on indicated tumor sections in different treatment groups; subpart K showing a schematic diagram illustrating extracellular adenosine production in the GBM TME; and subpart L showing graphical data depicting adenosine concentration in local brain tissues of mice in each treated group (Note: the data shown in FIG. 19 are for isolated pNK cells in at least triplicates; Data shown as mean±SEM; *p<0.05, **p<0.01).

While the present disclosure is susceptible to various modifications and alternative forms, exemplary embodiments thereof are shown by way of example in the drawings and are herein described in detail.

### BRIEF DESCRIPTION OF THE SEQUENCE LISTINGS

SEQ ID NO: 1 is an amino acid sequence of a protease-sensitive linker as follows:

GGGGSGGGGSGGGGS;

SEQ ID NO: 2 is an amino acid sequence of a cleavable peptide fragment as follows:

LSGRSDNH;

SEQ ID NO: 3 is an amino acid sequence of a “self-cleaving” P2A peptide as follows:

GSGATNFSLLKQAGDVEENPGP;

SEQ ID NO: 4 is a nucleic acid sequence of a primer as follows:

ACATCGCTCAGACACCATG;

SEQ ID NO: 5 is a nucleic acid sequence of a primer as follows: TGTAGTTGAGGTCAATGAAGGG; and

SEQ ID NO: 6 is an artificial amino acid sequence of at least one embodiment of an antigen binding domain or fragment thereof of the present disclosure that comprises a natural killer group 2 member D receptor (NKG2D) (hCD3zeta-hNKG2D-P2A-hDAP10/Flag):

MRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPRGWIRGRRSRHSWEMSEFHNYNLDLKKSDFSTRWQKQ
RCPVVKSKCRENASPFFFCCFIAVAMGIRFIIMVTIWSAVFLNSLFNQEV
QIPLTESYCGPCPKNWICYKNNCYQFFDESKNWYESQASCMSQNASLLKV
YSKEDQDLLKLVKSYHWMGLVHIPTNGSWQWEDGSILSPNLLTIIEMQKG
DCALYASSFKGYIENCSTPNTYICMQRTVGSGATNFSLLKQAGDVEENPG
PMIHLGHILFLLLLPVAAADYKDDDDKQTTPGERSSLPAFYPGTSGSCSG
CGSLSLPLLAGLVAADAVASLLIVGAVFLCARPRRSPAQDGKVYINMPGR
G;

SEQ ID NO: 7 is an artificial amino acid sequence of at least one embodiment of an antigen binding domain or fragment thereof of the present disclosure that specifically binds the cancer-associated antigen disialoganglioside (GD2) and comprises a scFv (anti-GD2 scFv-CD8H-hCD28-hCD3zeta):

EVQLLQSGPELEKPGASVMISCKASGSSFTGYNMNWVRQNIGKSLEWIGA
IDPYYGGTSYNQKFKGRATLTVDKSSSTAYMHLKSLTSEDSAVYYCVSGM
EYWGQGTSVTVSSGGGGSGGGGSGGGGSDVVMTQTPLSLPVSLGDQASIS
CRSSQSLVHRNGNTYLHWYLQKPGQSPKLLIHKVSNRFSGVPDRFSGSGS
GTDFTLKISRVEAEDLGVYFCSQSTHVPPLTFGAGTKLELKTTTPAPRPP
TPAPTIASQPLSLRPEACRPAAGGAVHTRGLDFACDIFWVLVVVGGVLAC
YSLLVTVAFIIFWVRSKRSRLLHSDYMNMTPRRPGPTRKHYQPYAPPRDF
AAYRSMRVKFSRSADAPAYQQGQNQLYNELNLGRREEYDVLDKRRGRDPE
MGGKPQRRKNPQEGLYNELQKDKMAEAYSEIGMKGERRRGKGHDGLYQGL
STATKDTYDALHMQALPPR;

SEQ ID NO: 8 is an artificial amino acid sequence of at least one embodiment of a CAR construct of the present disclosure comprising a first antigen binding domain or fragment thereof that specifically binds the cancer-associated antigen GD2 (SEQ ID NO: 7) operably linked to a second binding domain or fragment thereof specific for CD73 via a cleavable linker, and a third antigen binding domain or fragment thereof comprising NKG2D (SEQ ID NO: 6) (anti-CD73 scFv-linker-cleavable peptide-linker-GD2.CAR-NKG2D.CAR):

EVQLLESGGGLVQPGGSLRLSCAASGFTFSSYAYSWVRQAPGKGLEWVSA
ISGSGGRTYYADSVKGRFTISRDNSKNTLYLQMNSLRAEDTAVYYCARLG
YGRVDEWGRGTLVTVSSGGGGSGGGGSGGGGSQSVLTQPPSASGTPGQRV
TISCSGSLSNIGRNPVNWYQQLPGTAPKLLIYLDNLRLSGVPDRFSGSKS
GTSASLAISGLQSEDEADYYCATWDDSHPGWTFGGGTKLTVLGGGGSGGG
GSGGGGSLSGRSDNHGSSGTEVQLLQSGPELEKPGASVMISCKASGSSFT
GYNMNWVRQNIGKSLEWIGAIDPYYGGTSYNQKFKGRATLTVDKSSSTAY
MHLKSLTSEDSAVYYCVSGMEYWGQGTSVTVSSGGGGSGGGGSGGGGSDV
VMTQTPLSLPVSLGDQASISCRSSQSLVHRNGNTYLHWYLQKPGQSPKLL
IHKVSNRFSGVPDRFSGSGSGTDFTLKISRVEAEDLGVYFCSQSTHVPPL
TFGAGTKLELKTTTPAPRPPTPAPTIASQPLSLRPEACRPAAGGAVHTRG
LDFACDIFWVLVVVGGVLACYSLLVTVAFIIFWVRSKRSRLLHSDYMNMT
PRRPGPTRKHYQPYAPPRDFAAYRSMRVKFSRSADAPAYQQGQNQLYNEL
NLGRREEYDVLDKRRGRDPEMGGKPQRRKNPQEGLYNELQKDKMAEAYSE
IGMKGERRRGKGHDGLYQGLSTATKDTYDALHMQALPPRGSGATNFSLLK
QAGDVEENPGPMRVKFSRSADAPAYQQGQNQLYNELNLGRREEYDVLDKR
RGRDPEMGGKPQRRKNPQEGLYNELQKDKMAEAYSEIGMKGERRRGKGHD
GLYQGLSTATKDTYDALHMQALPPRGWIRGRRSRHSWEMSEFHNYNLDLK
KSDFSTRWQKQRCPVVKSKCRENASPFFFCCFIAVAMGIRFIIMVTIWSA
VFLNSLFNQEVQIPLTESYCGPCPKNWICYKNNCYQFFDESKNWYESQAS
CMSQNASLLKVYSKEDQDLLKLVKSYHWMGLVHIPTNGSWQWEDGSILSP
NLLTIIEMQKGDCALYASSFKGYIENCSTPNTYICMQRTVGSGATNFSLL
KQAGDVEENPGPMIHLGHILFLLLLPVAAADYKDDDDKQTTPGERSSLPA
FYPGTSGSCSGCGSLSLPLLAGLVAADAVASLLIVGAVFLCARPRRSPAQ
DGKVYINMPGRG.

In addition to the foregoing, the above-described sequences are provided in computer readable form encoded in a file filed herewith and herein incorporated by reference. The information recorded in computer readable form is identical to the written Sequence Listings provided above, pursuant to 37 C.F.R. § 1.821(f).

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the compositions, systems and methods hereof may comprise many different configurations, forms, materials, and accessories.

In the following description, numerous specific details are set forth in order to provide a thorough understanding of the present disclosure. Particular examples may be implemented without some or all of these specific details and it is to be understood that this disclosure is not limited to particular biological systems, which can, of course, vary.

Unless defined otherwise, all technical and scientific terms used herein have the same meaning as commonly understood by one of skill in the relevant arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise. Furthermore, unless specifically stated otherwise, the term “about” can allow for a degree of variability in a value or range, including a range of values plus or minus 10% for percentages and plus or minus 1.0 unit for unit values, for example, about 1.0 refers to a range of values from 0.9 to 1.1, or of a stated limit of a range. The term “substantially” can allow for a degree of variability in a value or range as well, for example, within 90%, within 95%, or within 99% of a stated value or of a stated limit of a range.

A “subject” or “patient” as the terms are used herein is a mammal, preferably a human, and is inclusive of male, female, adults, and children.

“Nucleic acid” refers to deoxyribonucleotides or ribonucleotides and polymers thereof in either single- or double-stranded form and complements thereof. The term encompasses nucleic acids containing known nucleotide analogs or modified backbone residues or linkages, that are synthetic, naturally occurring, and non-naturally occurring, have similar binding properties as the reference nucleic acid, and metabolized in a manner similar to the reference nucleotides.

The terms “polypeptide,” “peptide,” and “protein” are used interchangeably herein (unless expressly stated otherwise) to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

As used herein, “adenosinergic” means working on adenosine.

An “antibody fragment” as used herein means a portion of an intact antibody, preferably the antigen-binding or variable region of the intact antibody. Examples of antibody fragments include, for example, single-chain antibody molecules (scFv), nanobodies, F(ab′)₂, F(ab)₂, Fab′, Fab, Fv, or dAb. Regardless of structure, an antibody fragment as used herein binds with the same antigen that is recognized by the full-length antibody. For example, antibody fragments include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). “Single-chain antibodies,” often abbreviated as “scFv” consist of a polypeptide chain that comprises both a V_{H} and a V_{L} domain which interact to form an antigen-binding site. The V_{H} and V_{L} domains are usually linked by a peptide of 1 to 25 amino acid residues. Antibody fragments also include diabodies, triabodies, and single domain antibodies (dAb). While in the present disclosure reference is made to antibodies and various properties of antibodies, the disclosure applies to functional antibody fragments as well unless expressly noted to the contrary.

“Chimeric antigen receptors” (CARs, also known as chimeric T cell receptors) are synthetic constructs designed to be expressed in host T cells or NK cells and to induce an immune response against a specific target antigen (e.g., CD39/CD79) and cells expressing that antigen.

The CAR typically comprises an antibody fragment, such as a single chain antibody (scFv) or Fab fragment, incorporated in a fusion protein that also comprises additional components, such as a CD3-ζ or CD28 transmembrane domain and selective T-cell activating moieties, including the endodomains of CD3-ζ, CD28, OX40, 4-1BB, Lck, and/or ICOS. Various combinations of such elements may also be used. Accordingly, CARs may also be designed to transduce activation signals via co-stimulatory domains such as those utilizing immunoreceptor activation motifs (ITAMs) present in the cytoplasmic tails. Gene constructs utilizing an antigen-binding moiety afford the additional advantage of being “universal” in that they bind native antigen on the target cell surface in an human leukocyte antigen (HLA)-independent fashion and therefore do not need to be collected from a patient or a specific HLA-matched donor.

A CAR according to the embodiments of the present disclosure can be produced by any means known in the art, though preferably it is produced using recombinant DNA techniques. A nucleic acid sequence encoding the several regions of the CAR can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous NK cells. In other words, transduction or transfection can be performed to introduce the CAR expression constructs into NK cells, which may then be used to induce an immune response in the subject. Additionally or alternatively, the CRISPR/Cas9 genome editing technology and the like may also be employed to knockdown particular genes. Techniques for genetic manipulation of NK cells for cancer immunotherapy are generally known in the art.

Further, as noted above, CAR-NK cells may contain a targeting molecule, such as a scFV or Fab, that binds to a disease associated antigen (TAA) or to a hapten on a targetable construct. This avoids the problem that NK cells, unlike T cells, lack antigen specificity for targeting cells to be killed. The cell-targeting scFv or Fab may be linked via a transmembrane domain to one or more intracellular signaling domains to effect lymphocyte activation. Signaling domains used with CAR-NK cells may include, for example, CD3-ζ, CD28, and the like. The CAR constructs of the present disclosure may include any such constructs known in the art. A wide variety of CAR constructs have been reported and are commercially available.

An “antigen binding domain or fragment thereof” or a “binding domain or fragment thereof” of the present disclosure “that binds” a target of interest is one that binds the antigen/target with sufficient affinity such that the protein, binding domain, or engineered cell is useful as a diagnostic and/or therapeutic agent in targeting a protein or a cell or tissue expressing the antigen. With regard to the binding of a protein, binding domain, and/or engineered cell to a target molecule, the term “specific binding” or “specifically binds to” or is “specific for” a particular polypeptide or an epitope on a particular polypeptide target means binding that is measurably different from a non-specific interaction. Specific binding can be measured, for example, by determining by competition with a control molecule that is similar to the target. In at least one embodiment, “specifically binds” refers to binding of the antigen binding domain to its specified adenosine-producing enzyme target receptors (e.g., CD73 or CD39) and not other specified non-target receptors.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it is positioned so as to facilitate translation. Generally, “operably linked” means that the DNA sequences being linked are contiguous and, in the case of leader, contiguous and in a reading phase. However, enhancers do not necessarily have to be contiguous. Linking may be accomplished by ligation at convenient restriction sites. If such sites do not exist, synthetic oligonucleotide adaptors or linkers may be used in accordance with conventional practice.

“Percent (%) amino acid sequence identity” with respect to a reference to a polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity and not considering any conservative substitutions as part of the sequence identity. Alignment for purposes of determining percent amino acid sequence identity can be achieve din various ways that are within the skill of the art, for instance, using publicly available computer software. Those skilled in the art can determine appropriate parameters for aligning sequences, including any algorithms needed to achieve maximal alignment over the full length of the sequences being compared.

“Downregulation” or “down-regulated” may be used interchangeably and refer to a decrease in the level of a marker, such as a gene, nucleic acid, metabolite, transcript, enzyme, protein, or polypeptide, as compared to an established level (e.g., that of a healthy cohort or the subject of interest). “Upregulation” or “up-regulated” or “overexpressed” may also be used interchangeably and refer to an increase in the level of a marker, such as a gene, nucleic acid, metabolite, transcript, protein, enzyme, or polypeptide, as compared to an established level (e.g., that of a healthy control or the subject of interest). For example, relevant in the present application, CD73 may be overexpressed in a patient experiencing a solid tumor or other cancer as compared to a healthy control. Additionally or alternatively, a NK T cell may be engineered to upregulate the expression of NKG2D of the engineered T cell.

A “marker” or “biomarker” as the terms are used herein may be described as being differentially expressed when the level of expression in a subject who is experiencing an active disease state is significantly different from that of a subject or sample taken from a healthy subject. A differentially expressed marker may be overexpressed or underexpressed as compared to the expression level of a normal or control sample or subjects' baseline (i.e. downregulated). The increase or decrease, or quantification of the markers in a biological sample may be determined by any of the several methods known in the art for measuring the presence and/or relative abundance of a gene product or transcript. The level of markers may be determined as an absolute value, or relative to a baseline value, and the level of the subject's markers compared to a cutoff index. Alternatively, the relative abundance of the marker or markers may be determined relative to a control, which may be a clinically normal subject.

The terms “treatment” or “therapy” as used herein (and grammatical variations thereof such as “treat, “treating,” and “therapeutic”) include curative and/or prophylactic interventions in an attempt to alter the natural course of the individual being treated. More particularly, curative treatment refers to any of the alleviation, amelioration and/or elimination, reduction and/or stabilization (e.g., failure to progress to more advanced stages) of a symptom, as well as delay in progression of a symptom of a particular disorder. Prophylactic treatment refers to any of the following: halting the onset, reducing the risk of development, reducing the incidence, delaying the onset, reducing the development, and increasing the time to onset of symptoms of a particular disorder. Desirable effects of treatment include, but are not limited to, preventing occurrence or recurrence of a disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions of the present disclosure are used to delay development of a disease and/or tumor, or to slow (or even halt) the progression of a disease and/or tumor growth.

As used herein, the term “anti-tumor effective amount” refers to an effective amount of construct-expressing NK cells to reduce cancer cell or tumor growth or to decrease tumor volume or number of tumor cells in a subject. “An anti-tumor effective amount” can also refer to an effective amount of engineered NK cells or an engineered NK cell line to increase life expectancy or to alleviate physiological effects associated with the tumor or cancer.

As used herein, the phrases “therapeutically effective dose,” “therapeutically effective amount,” and “effective amount” means (unless specifically stated otherwise) a quantity of a polypeptide and/or engineered cells of the present disclosure which, when administered either one time or over the course of a treatment cycle, affects the health, wellbeing or mortality of a subject (e.g., and without limitation, a diminishment or prevention of effects associated with a cancerous condition). The a appropriate dosage or amount of a polypeptide, engineered cells, or other compound to be administered to a subject for treating a disease, condition, or disorder (including, without limitation, a cancerous condition such as a solid state tumor) as described herein will vary according to several factors including the type and severity of condition being treated, how advanced the disease pathology is, the formulation of the composition, patient response, the judgment of the prescribing physician or healthcare provider, whether one or more constructs are being administered, the route of administration, and the characteristics of the patient or subject being treated (such as general health, age, sex, body weight, and tolerance to drugs). Thus, the absolute amount of engineered cells included in a given unit dosage form can vary widely, and depends upon factors such as the age, weight and physical condition of the subject, as well as the method of administration.

A therapeutically effective amount is also one in which any toxic or detrimental effects of the therapeutic agent are outweighed by the therapeutically beneficial effects. In at least one embodiment, an anti-tumor effective amount may be a therapeutically effective dose.

Administered dosages for the engineered cells as described herein for treating cancer, a cancerous tumor, or other disease or disorder are in accordance with dosages and scheduling regimens practiced by those of skill in the art. Typically, doses >10⁹ cells/patient are administered to patients receiving adoptive cell transfer therapy. Determining an effective amount or dose is well within the capability of those skilled in the art, especially in light of the detailed disclosure provided herein.

The term “pharmaceutical composition” means a composition comprising one or more of engineered cells or engineered NK cell lines as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents, and dispensing agents (depending on the nature of the mode of administration and dosage forms).

The term “pharmaceutically acceptable” and grammatical variations thereof, as they refer to compositions, carriers, diluents, reagents, and the like, are used interchangeably and represent that the materials are capable of administration to or upon a mammal without undue toxicity, irritation, allergic response, and/or the production of undesirable physiological effects such as nausea, dizziness, gastric upset, and the like as is commensurate with a reasonable benefit/risk ratio. In other words, it is a material that is not biologically or otherwise undesirable—i.e. the material may be administered to an individual along with NK cells (and/or stem cells or iPSCs) modified to express the constructs of the present disclosure without causing any undesirable biological effects or interacting in a significantly deleterious manner with any of the other components of the pharmaceutical composition in which it is contained.

The term “isolated” means that the material is removed from its original environment, e.g., the natural environment if it is naturally occurring. For example, a naturally occurring NK cell present within a living organism is not isolated, but the same NK cell separated from some or all of the coexisting materials in the natural system is isolated.

Conventional treatments for glioblastoma (GBM), including without limitation surgery, radiation, and chemotherapy), have proven ineffective largely due to the severe immunosuppression characteristic of GBM. GBM is a particularly challenging tumor to treat due to its intratumor heterogeneity, whereby cells with varying molecular, genetic and epigenetic characteristics contribute to its ability to evade conventional treatments. Further, GBM is characterized by antigen escape variants that resist single antigen-targeting monotherapies.

The limitations of single antigen-based therapies have been recognized in clinical studies through tumor outgrowth variants. To date, CAR-NK cells that target single GBM antigens—EGRF, EGRFvIII, or ErbB2/HER2—have been limited to the use of NK cell lines, and the overall response rates have been disappointingly low and inconsistent. These results appear to mirror the clinical hurdles of single antigen targeted CAR-T therapies for GBM.

The stringent requirements for target selection place constraints on the targeting strategies. While combinatorial antigenic targeting with multi-targeted CAR-engineered cells have demonstrated some ability to address antigen escape in GBM, poor immune cell infiltration and immunometabolic reprogramming caused by the primarily hypoxic GBM TME has stymied durable responses. Further, and unsurprisingly, outgrowth of antigen escape variants has been recorded clinically with most GBM-associated antigens to date, resulting in immune evasion and resistance to treatment. The nonsustainable response has resulted in a lack of improvement in the overall survival (OS) of GBM patients treated with CAR-T therapies in Phase III trials.

While dual antigen-targeting or programmable, tumor-sensing CARs have been evaluated pre-clinically for GBM treatment solely in the context of adoptive T cell therapy, GBM employs mechanisms beyond antigen escape to avoid immune surveillance that such approaches do not address. Accordingly, dual-antigen programming alone is not sufficient for a clinically effective treatment. Indeed, treatment evasion by GBM is additionally fueled by a heavily immunosuppressive, hypoxic TME which provides a niche unfavorable to NK cell effector function. Further, the highly hypoxic nature of many tumors (like GBM, for example) also fuels the activity of ectonucleoside triphosphate diphosphohydrolase-1 (CD39), ecto-5′-nucleotidase (CD73) and other adenosinergic signaling variants (e.g., CD38, etc.) to produce the immunosuppressive metabolite adenosine, which leads to significant purinergic signaling-mediated impairment of NK cell activity.

In addition, GBM cells utilize autophagy to promote their growth, progression, and resistance to therapy. Finally, a subset of GBM cells, glioma stem-like cells (GSCs) also contribute to treatment resistance and are poorly recapitulated by convention GBM model cell lines, including U87MG. This complexity results in the need for varying therapeutic strategies that can act in concert or synergy to mount meaningful therapeutic responses.

The inventive concepts of the present disclosure generally relate to methods, compositions, and engineered peptides for the treatment of cancers, particularly GBM, through multifunctional engineered NK cells that not only target one or more cancer-specific antigens, but also, in at least one embodiment, inhibit autophagy such that the GBM is sensitized for the therapeutic treatment. Furthermore, certain embodiments of the present disclosure directly inhibit adenosinergic signaling. Accordingly, the compositions, systems, and methods of the present disclosure are the first multifunctional, engineered NK cell-based therapy for GBM that simultaneously targets multiple clinically relevant pathways of GBM progression including, for example, antigen escape, immunometabolic suppression, and/or poor intratumoral NK cell presence, using the novel engineered NK cells described herein. While the data of the present disclosure relates to GBM in particular, it will be understood by those of ordinary skill in the art that the inventive concepts hereof related to any cancer that expresses cancer-associated antigens and adenosine or an adenosine-intermediary.

In at least one exemplary embodiment, for example, a single NK-cell construct is provided that comprises at least bifunctional constructs (e.g., CARs), namely, at least a first construct for encoding a protein specific to a cancer-associated antigen (for example, and without limitation, disialoganglioside (GD2)) and at least a second construct for targeting cognate ligands to a NK activating receptor (for example, and without limitation, the potent activating NK cell receptor natural killer group 2D (NKG2D)). When expressed in a NK cell, these two binding domains may be coupled with one or more stimulatory or costimulatory domains (i.e. signaling domains) of the NK cell, which may comprise a transmembrane domain and/or an intracellular domain. One or more of such constructs may be expressed in a single NK cell as desired. Use of at least two different CARs in this context avoids antigen escape of the cancer cells seen with conventional treatments.

Furthermore, at least one of the constructs may further be engineered to impair immunosuppressive purinergic/adenosinergic signaling, for example and without limitation, through the inclusion of one or more CD73-, CD39-, or CD38-blocking antibody fragments operably linked with the construct via a cleavable linker. For example, in at least one embodiment, the first CAR may be coupled with anti-CD73scFv via a cleavable linker. In this manner, in operation, the antibody and/or fragment is released wholly independently of the CAR-based signaling and/or activation. The released antibody/fragment then functions to inhibit the activity of the adenosine producing cell surface protein or adenosine-intermediary producing cell surface protein of the cancer cells thereby decreasing the local concentration of extracellular adenosine which, in turn, impairs immunosuppressive purinergic signaling. In at least one exemplary embodiment, the release of the linker is tumor-sensitive and, thus, cleavable by the activity of proteases that are upregulated in the TME. This results in the localized release of the adenosine-blocking antibody fragment in the TME and, thus, avoids systemic toxicities.

Still further, to address the insufficient homing of NK cells into the tumor bed caused by GBM in particular, and to sensitize the cancer cells to immune cell treatment, embodiments of the present disclosure may also impair and/or disable autophagy through administration of at least one additional therapeutic treatment. In at least one embodiment, the additional therapeutic treatment comprises administering to the patient one or more pharmacological autophagy inhibitors such as chloroquine (CQ) and the like. Additionally or alternatively, the at least one additional therapeutic treatment comprises targeting one or more genes of the patient associated with the autophagy pathway through generating genetic knockdown patient-derived GBM cells. Especially when used in conjunction with the engineered NK cells of the present disclosure, the additional therapeutic treatment can achieve a potent reorganization of the GBM milieu and, thus, substantially enhance NK cell infiltration through the engagement of the CCL5 and CXCL10 chemokine axes.

Brief descriptions of the relevant cellular pathways and mechanisms will be provided to aid in understanding of the inventive concepts hereof, followed by a detailed description of the present constructs, compositions, and novel methods provided herein.

### Adenosine

Contributing to the pathogenesis of solid tumors are elevated concentrations of adenosine, a consequence of anaerobic glycolysis in hypoxic solid tumor cores. In solid tumors, ATP is abundantly released in the extracellular space where its concentration can reach a few hundred micromole per liter, a concentration more than a thousand times higher than in healthy tissues. This phenomenon is mainly due to cell death in the tumor core and to metabolic or hypoxic stress and pro-inflammatory signals that stimulate active export of ATP. In the TME, extracellular ATP acts as a danger signal involved in the recruitment of innate immune cells and in the priming of anti-tumor activity; however, at the same time the extracellular ATP is degraded into immunosuppressive adenosine via the concerted enzymatic activity of at least CD39 and CD73, as well as CD38. As a consequence, in various solid tumors, accumulation of extracellular adenosine followed by engagement of the adenosine receptors on tumor-reactive NK cells is a highly immunosuppressive mechanism that drives tumor growth.

CD39 and CD73 are ecto-nucleoside triphosphoate diphosphohydrolases, which are anchored cell surface proteins, and exhibit a catalytic site facing the extracellular space. CD38 and CD157 are alternative pathways that are also surface molecules with an extracellular catalytic domain, except theirs consists of ADP ribosyl-cyclases. Expression of these ectoenzymes by solid tumors such as GBM and in the TME results in the production of extracellular adenosine.

CD38 and CD157 are part of the same family of NADase/ADPR cyclase enzymes. CD38 is a surface glycoprotein characterized by a relatively large extracellular domain that harbors the catalytic site. CD157, on the contrary, is attached to the membrane via a glycosylphosphatidylinositol anchor. The extracellular domain of both molecules contains conserved critical residues. They both metabolize nicotinamide dinucleotide (NAD⁺), which also affects purinergic receptors and converge on adenosine generation with profound effects generating immune effectors cells (e.g., NK cells) towards tolerance. Indeed, extracellular NAD⁺ can be degraded by an integrated network of ectonucleotidases, including CD38 and CD157, which generate intermediates that modulate signaling and activate immunoregulatory circuits. Extracellular adenosine can be generated from NAD+ through to the coordinated action of CD38, which generates ADP ribose (ADPR) and PC-1 (ectonucleotide pyrophosphatase/phosphodiesterase family member 1), which generates AMP. Similar CD38, CD157 generates cADPR and subsequent ADPR when incubated with NAD⁺.

In human peripheral blood, both CD39 and CD73 are typically expressed on about 2-5% of NK cells within non-malignant blood cells. As such, expression of both CD39 and CD73 is virtually absent from circulating human NK cells in healthy individuals. However, significant expression of CD39 by human tumors and infiltrating immune cells has been widely described, which is associated with generation of adenosine that has an inhibitory role on effector anti-tumor immunity and exposure to proinflammatory cytokines, oxidative stress and hypoxia. Likewise, expression of CD73 remains at constitutively high levels on many types of cancer cells. High CD73 expression has been shown to be correlated with unfavorable clinical outcomes, which is consistent with the immunosuppressive role of adenosine. The expression of CD38, CD73, and/or CD157 may also be upregulated, especially in a TME that is hypoxic.

Accordingly, CD39 and CD73 are overexpressed on many solid tumor cells—GBM in particular—and implicated in the promotion of cancer progression through upregulation of adenosine signaling following dephosphorylation of extracellular AMP. As described herein, adenosinergic signaling interferes with the trafficking and activities of NK cells due to the heterologous desensitization of chemokine receptors and reduced proinflammatory cytokines and inhibits the exocytosis of cytotoxic NK granules. This creates a pro-angiogenic niche supporting tumor development.

Adenosine-induced immunosuppression can be alleviated by antibody-mediated blockade of CD73 or the variants thereof; however, this alone relies on the recruitment of NK cells to hypoxic tumor niches. Conventional efforts have not targeted adenosinergic signaling in conjunction with NK-based immunotherapy.

### NK Activity

NK cells, specialized effectors of the innate immune system, can respond rapidly to cancer cells due to expression of germline-encoded activating receptors capable of directly binding to pathogen-derived or stress-induced self-antigens. The activity of NK cells is controlled by a balance of signals from a repertoire of activating and inhibitory receptors. Activating receptors include, without limitation, natural cytotoxic receptors (NCRs), natural killer group 2 member D (NKG2D), CD16 (FcγRIIIA), FasL, TRAIL, and co-stimulatory receptors such as LFA-1, CD244 (2B4), and CD137 (41BB). These activating cell surface receptors have the capacity to trigger cytolytic programs, as well as cytokine and chemokine secretion via intra-cytoplasmic ITAMs such as 2B4, 41BB, and/or via other transmembrane signaling adaptors. As used herein, the term “signaling domain” means and includes stimulatory and costimulatory domains unless otherwise specified.

Conversely, inhibitory NK cell receptors predominantly recognize cognate MHC class I protein and provide self-tolerance toward healthy cells. Cells with absent or reduced expression of MHC class I protein, as often observed after transformation or viral infection, are unable to trigger sufficient inhibitory signals and become susceptible to NK cell attack.

Upregulated expression of ligands for activating NK cell receptors can render cells sensitive to NK cell attack. Once such activating receptor is the C-type lectin-like receptor NKG2d. NKG2d receptor is expressed in NK cells as well as many T cells, such as NKT cells, CD8+ T cells, and γδT cells. However, in T cells, the NKG2D usually acts only as a costimulatory receptor and does not directly mediate cytotoxicity, which is different from NK cells.

Expression of NKG2D ligands (often expressed in tumor cells) is generally regarded as a “danger signal,” marking cells for immune attack, and activating NK cells by binding to the NKG2D receptor. Indeed, ex vivo studies with human cells and in vivo tumor models in mice demonstrated that expression of NKG2D ligands on tumor cells results in an increased susceptibility to NK cell attack. Where the immune system is properly functioning, ligation of NKG2D on NK cells serves to promote NK cell activation and influence the adaptive immune response; however, there are various mechanisms (especially with GBM) that inhibit the action of NKG2D receptor/NKG2D ligand to enable immune escape.

As discussed herein, adenosine signaling results in downregulation of receptor expression on NK cells (for example, and without limitation, it has been established that adenosine downregulates NKG2D on cytokine-primed human NK cells). In addition to extracellular adenosine concentrations, the expression of NKG2D receptor on NK cells can be regulated by a variety of other factors, including changes in cellular activity factors and the physicochemical features of the TME (such as, for example, hypoxia). The TME is composed of a variety of cells and molecules, including tumor-associated fibroblasts, tumor-associated macrophages, Tregs, immunoregulatory enzymes (e.g., arginase and cyclooxygenase-2), and immunosuppressors (e.g., interleukin-10 (IL-10), transforming growth factor-β (TGF-β), vascular endothelial growth factor (VEGF), prostaglandin E₂ (PGE₂), and programmed death ligand 1). Tumor cells (such as GMB cells) and immunosuppressive cells express or secrete podocalyxin-like protein 1 (PCLP₁) activin-a, indoleamine-pyrrole 2, 3-dioxygenase (IDO), PGE₂, TGF-β, and macrophage migration inhibitory factor (MIF) in the TME to mediate NKG2D downregulation.

Furthermore, hypoxia is an important feature of the TME that can directly or indirectly induce the secretion of immunosuppressive molecules, such that NK cells lose the ability to upregulate NKG2D expression through IL-2 and other cytokines. Under hypoxic conditions, tumor cells can secrete a variety of chemokines to recruit immunosuppressive cells that secrete cytokines, thereby downregulating NKG2D expression.

### Constructs and Related Methods

The inventive constructs, engineered NK cells and NK cell lines, compositions and methods of the present disclosure uniquely target at least one, or a combination of, cancer antigen(s) and/or NK activating receptors, and further provides a releasable, soluble antibody fragment that impairs immunosuppressive purinergic signaling by blocking the activity of an adenosine producing cell surface protein or an adenosine-intermediary producing cell surface protein of the cancer/tumor cell. Where multiple cancer antigens and/or activating receptors are employed, this limits the tumor's ability for antigen escape. Further, in at least one embodiment where the adenosine-blocking, soluble antibody fragment is spatially-controlled, it releases only when within the TME to allow for the sustained and non-toxic release thereof. This, accordingly, avoids systemic toxicity issues seen in other conventional therapies.

The novel approaches of the present disclosure uniquely combine the specificity of engineered NK cells with the immune engagement induced by a blockade of adenosine producing enzymes (e.g., anti-CD73). Still further, as is described in additional detail below, such treatments may be combined with the administration of one or more autophagy inhibitors to further sensitive the tumor and further promote treatment efficacy. Accordingly, the constructs, engineered NK cells, pharmaceutical compositions and resulting therapies of the present disclosure yield combination immunotherapy modalities that can target multiple clinically-recognized mechanisms of GBM progression simultaneously while avoiding toxicity.

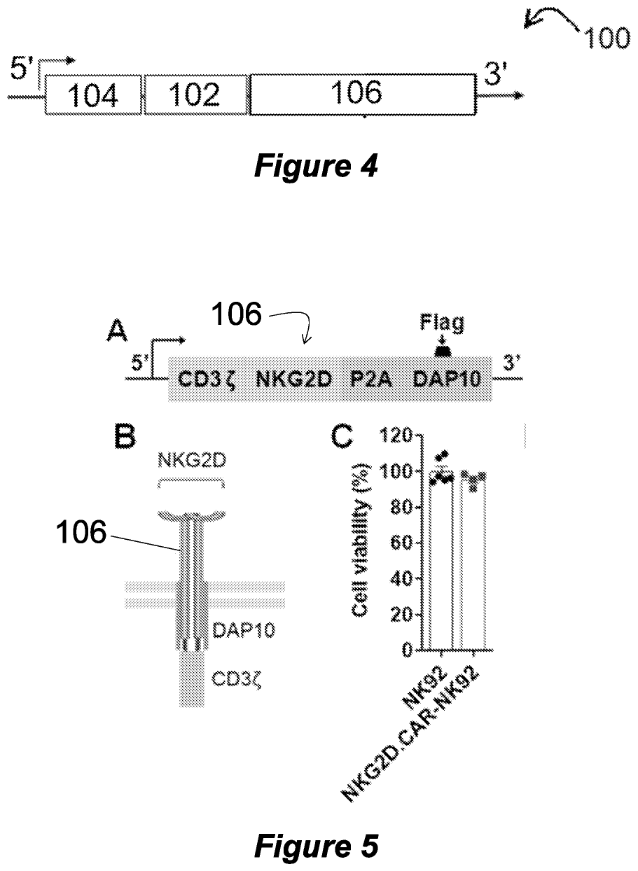

Now referring to FIG. 4, at least one exemplary embodiment of a synthetic genetic construct 100 is provided. The genetic construct 100 is engineered so that the NK cells and NK cell lines that express it (achieved via bioengineering and other known modalities) express at least one domain and/or receptor that are not normally expressed on the surface of native NK cells. The binding of these modified NK cells and NK cell lines to ligands on target cells, such as tumor/GBM cells, is through new domains not present in native NK cells. In at least one embodiment, the construct 100 may comprise one or more CAR constructs. In at least one exemplary embodiment, the construct 100 comprises a first CAR construct and a second CAR construct.

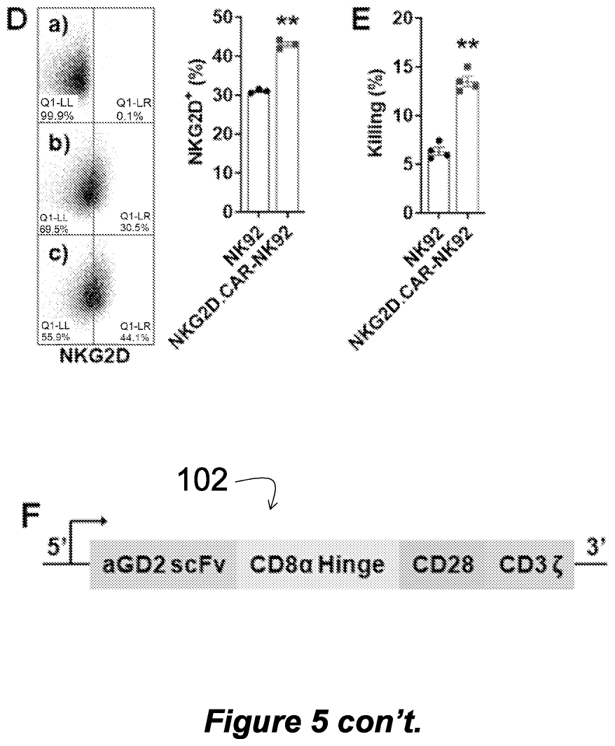

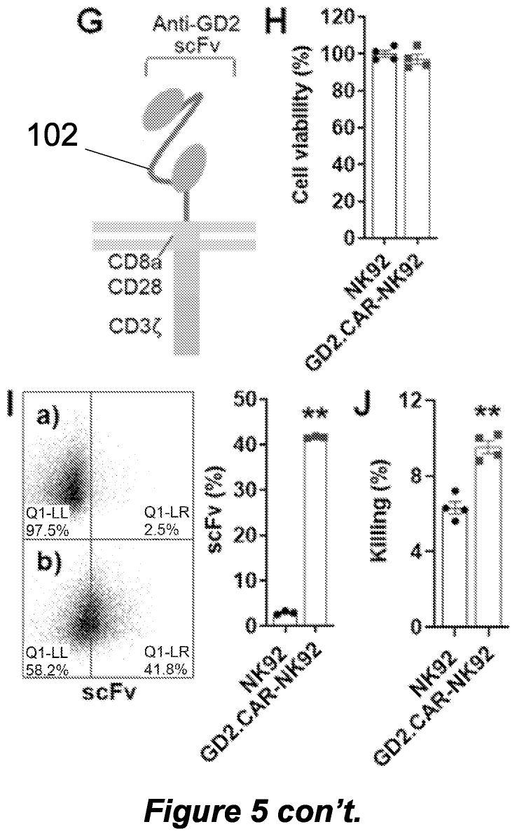

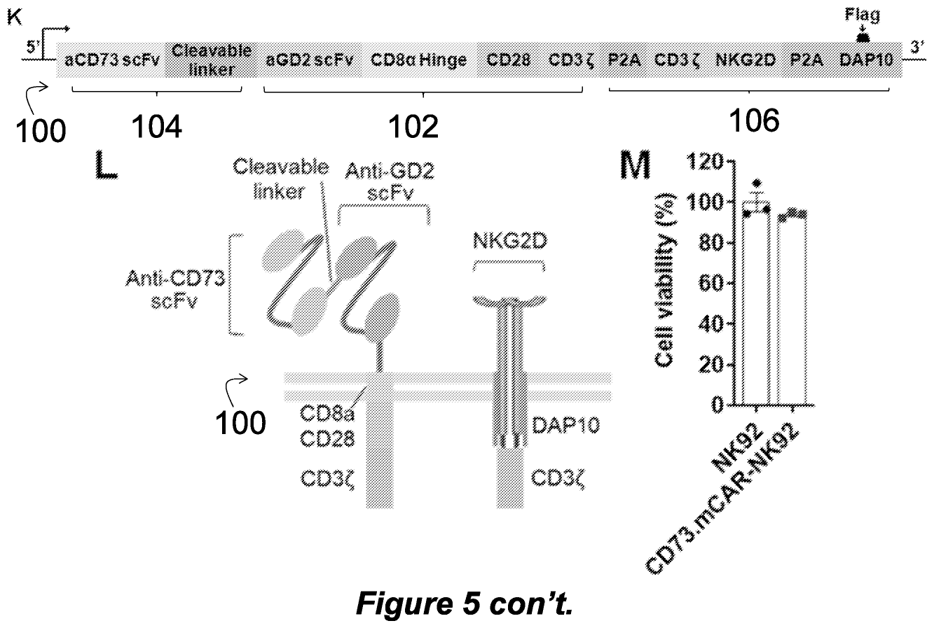

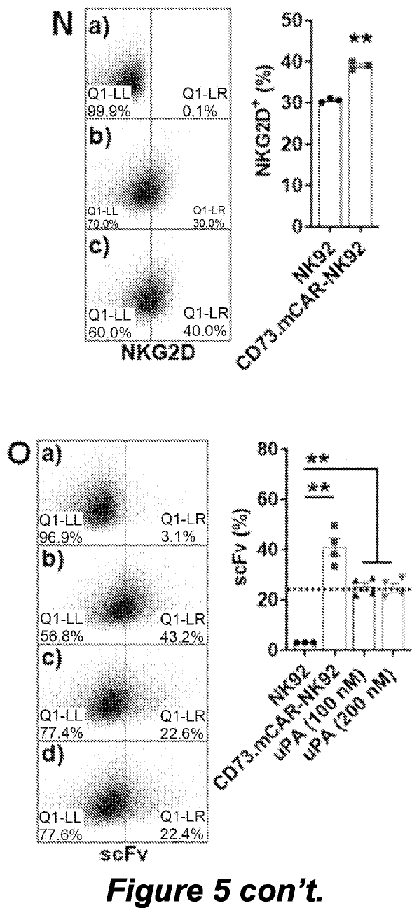

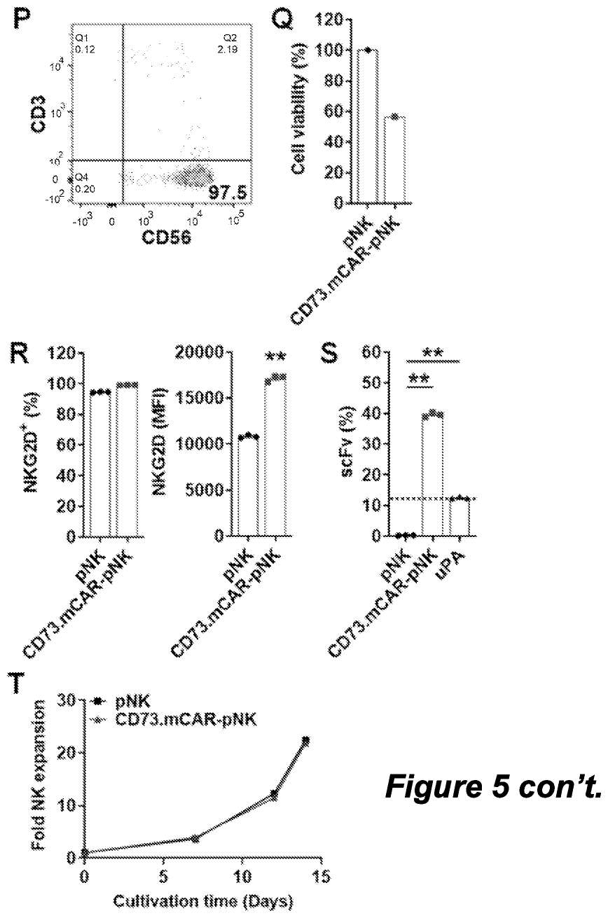

In perhaps its simplest form, the genetic construct 100 comprises a first construct comprising a first sequence that encodes at least a first binding domain or fragment thereof 102 operably linked to a second sequence encoding at least a second binding domain or fragment thereof and a cleavable linker 104. The first binding domain or fragment thereof 102 may comprise a NK activating receptor (e.g., NKG2D; SEQ ID NO: 6) or a protein specific for a cancer-associated antigen (such as, for example, GD2; SEQ ID NO: 7). FIG. 5, subparts F and G, illustrate in schematic at least one embodiment of a first binding domain or fragment thereof 102 encoded by the first sequence.

In at least one exemplary embodiment, the first construct is further engineered to also encode a second binding domain or fragment thereof 104 that is specific for an adenosine or adenosine-intermediary producing cell surface protein of a target cell. For example, such adenosine or adenosine-intermediary producing cell surface protein may comprise CD39, CD73, CD38 or any other cell surface protein of a target cell that produces adenosine or an intermediary thereof. In at least one exemplary embodiment, the adenosine or adenosine-intermediary producing cell surface protein comprises CD73 and the second binding domain or fragment thereof 104 comprises an anti-CD73 fragment such as an anti-CD73 scFv (see FIG. 5, subpart K).

Notably, the second binding domain or fragment thereof 104 further comprises a cleavable linker. Such cleavable linker may comprise any such linker known in the art and, in at least one exemplary embodiment, comprises a linker that is cleavable by one or more proteases present within the TME. As described above in detail, cancer cells produce adenosine through CD73 and other surface proteins. As the second binding domain 104 may be specific for such adenosine or adenosine-intermediary producing tumor cell surface proteins, when its linker is cleaved in the TME, the resulting soluble antibody fragment is released from the engineered NK cell and allowed to bind the adenosine (or adenosine-intermediary) producing tumor cell surface protein.

The genetic construct 100 may further comprise a second construct as shown in FIG. 5, subparts K and L. Similar to the first construct, the second construct may comprise a second sequence that encodes at least one binding domain (or fragment thereof) 106 which comprises a NK activating receptor or a protein specific for a cancer-associated antigen. FIG. 5, subparts A and B, illustrate in schematic at least one embodiment of the third binding domain or fragment thereof encoded by the second sequence.

One or both (where applicable) of the first and third sequences may encode signaling domain(s) of a NK cell such that they can promote cytotoxic and/or cytolytic activity of the engineered cell or cell line upon activation (see, for example and without limitation, CD (in FIG. 5, subparts A and F, and DAP10 in FIG. 5, subpart A). The first and third binding domains 102, 106 can comprise complimentary determining regions, variable regions, and/or antigen binding fragments thereof, as desired. It will likewise be appreciated that various linkers, spacers, scFv, hinges, and/or protein complexes may be additionally encoded by one or both of the first and second sequences as may be required to achieve the desired results.

While the first sequence and the third sequence are represented in FIG. 5 as comprising an activating receptor 102 and a protein specific for a cancer-associated antigen 106, respectively, the first and third sequences may both encode proteins for cancer-associated antigens or both encode NK cell activating receptors (i.e. there need not necessarily be one of each type). Furthermore, the second sequence encoding the second binding domain or fragment thereof 104 may be operatively linked to a NK cell activating receptor of the first domain 102. Where the first and third sequences both encode an antigen or a receptor 102, 106, in an exemplary embodiment, they may encode two different cancer-specific antigens or two different NK cell activating receptors; however, while often desirable, this is not required.

FIG. 5, subparts K and L illustrate schematics of at least one exemplary embodiment of a genetic construct of the present disclosure that comprises the first, second, and third binding domains (or fragments thereof) 102, 104, 106. There, by way of non-limiting example, the first binding domain 102 comprises anti-GD2 scFv fragment operatively linked with a CD8α Hinge, and two signaling domains (CD28 and CD3ζ) (collectively, GD2-CAR; SEQ ID NO: 7), the second binding domain 104 comprises an anti-CD73 scFv fragment is linked to the first binding domain 102 via a cleavable linker (SEQ ID NO: 1), and the third binding domain 106 comprises NKG2D, two signaling domains (DAP10 and CD3ζ), and at least one P2A self-cleaving peptide (NKG2D-CAR; SEQ ID NO: 6), where the GD2-CAR is associated with the NKG2D-CAR through one of the self-cleaving P2A peptides (SEQ ID NO: 4). In at least one exemplary embodiment, the construct 100 encodes at least a 80%, 85%, or 90% sequence identity to SEQ ID NO: 8.

The target cell may comprise any cell that produces cancer-associated antigens, NKG2D or other ligands cognate with an NK activating receptor, and/or adenosine or an intermediary thereof through a cell surface protein, for example, and without limitation, a cancer cell or otherwise malignant cells within a TME. GD2 and ligands to NKG2D, in particular, are widely expressed on human GBM and, as such, are referred to in the present disclosure by way of non-limiting example. However, other cancer-associated antigens may be encoded in the constructs of the present disclosure, such as, without limitation, ganglioside G3 (GD3), Her 2 (p185), CD19, CD20, CD56, CD123, CD22, CD30, CD33, CD171, CS-1, C-type lectin-like molecule-1; EpCAM, G250, proteoglycans, GD3, GD2, MHC II, TAG-72, milk mucin core protein, Lewis A antigen, tyrosine-protein kinase transmembrane receptor (ROR1), c-met, epidermal growth factor receptor (EGFR), EGFR variant III, and carcinoembryonic antigen (CEA), and certain antigens may be particularly associated with certain types of cancers.

Similarly, while NKG2D is shown in FIG. 5 as a representative NK cell activating receptor, any NK activating receptor that has cognate ligands presented on the desired target cell may be encoded in construct 100 including, without limitation, NKp30, NKp46, NKp40, DNAM-1, and the like. In certain embodiments, the constructs and resulting NK cells may be engineered specifically for a particular cancer-type by, for example, comprising a first sequence 102 and/or third sequence 106 that encodes a protein specific for an antigen or a cognate ligand that is predominantly expressed in the cancer of interest.

Additionally, certain embodiments of the construct 100 that encode a NK activating receptor may additionally be engineered for upregulated expression using techniques known in the art. For example, in at least one exemplary embodiment, one or more of the sequences encoding a binding domain or fragment thereof is engineered to encode upregulated expression of the NKG2D as compared to expression of NKG2D in a wild-type NK cell (i.e. expression of such receptor in the typical form of a NK cell at it occurs in nature). Such engineered upregulated expression is particularly useful when the engineered NK cells are employed to treat GBM and other tumors with a hypoxic TME, as it increases local NKG2D available.

As many of the cancer-associated antigens and cognate ligands of NK activating receptors are upregulated in cancer cells (and in particular GBM) and the TME, the inclusion of a protein specific for such antigens in one or more of the binding domains or fragments thereof 102, 106 in the construct 100 allows for the resulting engineered NK cells to directly target and recognize cancer and other such cells. To this end, the present constructs 100 enhance specificity and allow for the direct targeting and engagement of tumor, cancer and other malignant cells safely.

The binding domains 102, 104, 106 may further comprise one or more single-chain variable fragment (scFv) sequences or other antibody fragments such as nanobodies, which are fusion proteins between the variable regions of the heavy (V_{H}) and light (V_{L}) chains of immunoglobulins, connected with a shorter linker peptide of about ten to about 25 amino acids.

The specific configuration of the scFv or other antibody fragments may be selected based on desired properties of the resulting peptide (e.g., rich in glycine for flexibility, as well as serine or threonine for solubility). As is known in the art, the scFv or another antibody fragment can either connect the N-terminus of the V_{H} with the C-terminus of the V_{L}, or vice versa. The protein retains the specificity of the original immunoglobulin, despite removal of the constant regions and introduction of the scFv or other antibody fragments.

Accordingly, in at least one embodiment, the second binding region or domain 104 and the first binding region or domain 102 each comprise a a scFv fragment derived from a particular mouse, or human, or humanized monoclonal antibody or pursuant to other known sources and known methodologies. The fragment can also be any number of different antigen-binding domains of an antigen-specific antibody. In a more specific embodiment, the fragment is an antigen-specific scFv (e.g., αCD73 scFv or αGD2 scFv) encoded by a sequence that is optimized for human codon usage for expression in human NK cells. In at least one exemplary embodiment, the first sequence of the has at least a 80%, 85%, or 90% sequence identity to SEQ ID NO: 7, and the binding domain or fragment thereof 102 that it encodes CD73 scFv has at least a 80%, 85%, or 90% sequence identity to SEQ ID NO: 2.

Referring back to the one or more signaling domains that may be encoded by the construct 100, the first and/or third binding regions or domains 102, 106 may be operably linked thereto (directly or via a hinge region as described below) and, in fact the first binding domain may itself comprise a signaling domain (e.g., CD28, 4-1BB, DAP10). The one or more signaling domains may, for example, comprise an NK activator receptor or receptor complex capable of triggering the cytolytic and cytotoxic programs of the NK cell upon the associated binding domain or fragment thereof binding a target cell. For example, the signaling domain may comprise a CD28, CD3ζ, or DAP10 signal molecule or any other signaling domain known in the art. In certain embodiments, the signaling domains may comprise FcγRIIIA, FasL, TRAIL, 4-1BB, OX40, LFA-1, CD244, CD137, or the NKG2D-DAP10 receptor complex. Furthermore, the signaling domains may also comprise additional other costimulatory domains including, without limitation, one or more of DAP12, NKp46, NKp44, NKp30, and DAP10.

In application, engagement of at least the first and optionally third binding domains 102, 106 of the construct 100 with the target cell promotes signaling through the signaling domains of the engineered NK cell, resulting in activation of ITAM motifs on CD3 adaptor chains and NK cell-mediated cytotoxicity against solid tumor and other adenosine producing or adenosine-intermediary producing targets. Accordingly, when the engineered NK cell directly targets and binds a GD2- and/or NKG2D-ligand-producing surface cell protein (on a solid tumor, for example), signals are sent to the engineered NK cell via the signaling domains to trigger cytolysis and/or cytotoxicity mechanisms of the target (cancer) cell that it has bound.

Optionally, the construct 100 can additionally include a hinge domain (see FIG. 5, labeled “CD8a Hinge”) positioned within the binding domains 102, 106 at a location, for example and without limitation, between the αGD2 scFv and the signaling domains CD28 and CD3 of the third binding domain 106. A hinge domain may comprise one or more sequences that encode linkers or spacers and may be included in the construct, for example, to provide sufficient distance between the first/third binding domains 102, 106 and the membrane and/or cell surface. Additionally or alternatively, a hinge domain may be included (and/or configured) to facilitate a desired tertiary structure and/or alleviate possible steric hindrance that could adversely affect antigen binding or effector function of the modified NK cells. In this manner, the hinge domain can be used and/or manipulated for optimal expression in human cells.

Additionally or alternatively, additional intracellular signaling domains may be added to the construct 100 to enhance killing stimulus (i.e. further bolster the NK-mediated cytotoxicity of the resulting engineered NK cells). For example, human CD3ζ intracellular domains can be operably linked with both the first and third binding domains 102, 106 as shown in subpart L of FIG. 5. Other cytoplasmic domains may also be employed as desired, with one or multiple of such cytoplasmic domains fused together for additive or synergistic effect, if desired.

In operation, the binding domains 102, 104, 106 targets the cell(s) of interest and, when the binding domains 102, 106 bind the targeted cell, the engineered NK cell signals via the signaling domains to trigger cytolysis and/or cytotoxicity of the target cell. Further, where at least one of the binding domains or fragments thereof comprises an upregulated activating receptor (e.g., NKG2D), this enhances the anti-tumor responses of the NK cells and reduces not only the local concentration of extracellular adenosine, but also the immunosuppression of the NK cell activation. This is further bolstered by release of the second binding domain 104. As previously described, hen the second binding domain 104 is in the TME, the proteases therein cause the linker that tethers the second domain/fragment 104 to the first domain 102 to cleave, thereby releasing the fragment into the TME to initiate a local adenosine or adenosine-intermediary activity blockade in the target cell. In particular, the combination of increased NKG2D activity and the localized CD73 target blockade is highly effective in achieving controlled and localized responsiveness in GBM and other cancers.

Accordingly, the construct 100 allows for a multi-prong attack on the complex network of pathways that promote GBM and other cancer progression. Incorporating two or more (in an exemplary embodiment, three) targets into a single construct, one of which is releasable, enables the engineered NK cells of the present disclosure to not only activate and enhance the anticancer immune response, but also suppress GBM's evasion tactics such as antigen escape.

These inventive techniques are uniquely advantageous over conventional approaches. Primarily, allogenic stem cells and NK cells cause no graft versus host disease, making their widespread, off-the-shelf use feasible. Mature NK cells have a relatively limited lifespan, permitting effective antitumor activity while reducing the probability of long-term adverse events such as on-target/off-tumor effects. Further, expression of the present constructs prevent the systemic toxicity typically seen with CD73 and similar antibodies because it is locally released, and also rescue the downregulation of activating receptors induced by suppressive TME mechanisms such as hypoxia.

The constructs according to the embodiments can be prepared using conventional techniques. Because, for the most part, natural sequences may be employed, the natural genes may be isolated and manipulated, as appropriate, to allow for the proper joining of the various components. For example, the nucleic acid sequences can be isolated by employing the polymerase chain reaction (PCR), using appropriate primers that result in deletion of the undesired portions of the gene. Alternatively, restriction digests of cloned genes can be used to generate the chimeric construct. In either case, the sequences can be selected to provide for restriction sites that are blunt-ended or have complementary overlaps.

The various manipulations for preparing the constructs hereof can be carried out in vitro and in particular embodiments the construct is introduced into vectors for cloning and expression in an appropriate host using standard transformation or transfection methods. Thus, after each manipulation, the resulting construct from joining of the DNA sequences is cloned, the vector isolated, and the sequence screened to ensure that the sequence encodes the desired transgene and expression control sequences. The sequence can be screened by restriction analysis, sequencing, or the like as desired.

Vectors of the embodiments presented herein may further employ eukaryotic promoters as is known in the art. Also, the vectors may contain a selectable marker, if for no other reason, to facilitate their manipulation in vitro. In other embodiments, the transgene can be expressed from mRNA in vitro transcribed from a DNA template.

In an exemplary nucleic acid construct (polynucleotide) employed according to the embodiments, the promoter is operably linked to the nucleic acid sequence encoding a transgene of the embodiments, i.e., they are positioned so as to promote transcription of the messenger RNA from the DNA encoding the single-agent construct. The promoter can be of genomic origin or synthetically generated. Alternatively, a number of well-known viral promoters are also suitable.

For expression of a construct of the present disclosure in NK cells or an NK cell line, the naturally occurring or endogenous transcriptional initiation region of the nucleic acid sequence encoding the transgene can be used to generate the desired expression in the target host. Alternatively, an exogenous transcriptional initiation region can be used that allows for constitutive or inducible expression, wherein expression can be controlled depending upon the target host, the level of expression desired, the nature of the target host, and the like.

Likewise, in some cases, a leader and/or signal sequence added to the N-terminus specific for human protein expression directing the construct to be encoded by the transgene to the cell surface may be used.

Isolated nucleic acid segments and expression cassettes incorporating the DNA sequences of the constructs of the present disclosure are also provided. One of skill in the art will appreciate that such constructs may be employed with known gene modification techniques, including viral transduction, mRNA or DNA electroporation, and other viral and non-viral transduction and transfection techniques, to achieve engineered NK cells and/or an engineered NK cell line that expresses the constructs described herein.

Methods of making and/or expanding the engineered NK cells of the present disclosure are also provided. In at least one embodiment, a polynucleotide that encodes a construct provided herein can be introduced into a subject's own cells (or into cells from a different donor subject) using conventional transfection and/or transducing methods, either in a suitable vector or vector-free. Methods of stably transducing or transfecting NK cells by electroporation or otherwise are known in the art. In further aspects, the present constructs can be introduced into cells using a transposon-based system to mediate integration of the construct into genomic DNA of the cells, a non-viral vector, or a viral vector (e.g., a retroviral vector, adenoviral vector, adeno-associated viral vector, or lentiviral vector). Furthermore, in at least one embodiment, the CAR may be modified to facilitate uptake by the NK cells and, thus, expression of the construct-derived fusion protein in NK cells.

Sources of native NK cells may include both allogeneic and autologous sources. In some cases, NK cells may be differentiated from stem cells or induced pluripotent stem cells (iPSCs). For example, a construct as described herein can be expressed in stem cells or iPSCs, which can then be differentiated into NK cells using methods known to one skilled in the relevant arts. Thus, a cell for engineering according to the embodiments hereof can be isolated from umbilical cord blood, peripheral blood, human embryonic stem cells, or iPSCs.

In other embodiments, the NK cells are primary human NK (pNK) cells, such as NK cells derived from human peripheral blood mononuclear cells or umbilical cord blood. In at least one exemplary embodiment, the engineered NK cells may be produced from recurrent and primary patient-derived cells pursuant to methods known in the art. Alternatively, the engineered NK cell(s) and/or engineered NK cell line expressing the constructs of the present disclosure can be produced from a standardized cell population to provide a homogenous NK cell population that can be grown to clinical scale.

The NK cells, stem cells, pNK cells, or iPSCs modified to express a construct described herein may be formulated into a pharmaceutical composition along with a “carrier” for delivery to a subject having a condition at least partially characterized by cells that can be targets of NK cytotoxicity (e.g., adenosine overexpressing disease state). As used herein, “carrier” includes any solvent, dispersion medium, diluent, antibacterial, coating, vehicle, and/or antifungal agent, isotonic agent, absorption delaying agent, buffer, carrier solution, suspension, colloid, and the like. The use of such media and/or agents is well known in the art. Except insofar as any conventional media or agent is incompatible with the active ingredient, its use in the pharmaceutical compositions hereof is contemplated. Supplementary active ingredients can also be incorporated into the compositions.

Furthermore, the pharmaceutical composition of the present disclosure (e.g., comprising a engineered cells expressing a construct hereof) can be used alone or in combination with other well-established agents useful for treating cancer and/or solid tumor cancers. In at least one exemplary embodiment, one or more pharmaceutical compositions of the present disclosure may be administered to a single patient in conjunction with one or more autophagy inhibitors.

As discussed in the examples below in detail, it has been newly determined that the homing of the novel multifunctional engineered NK cells of the present disclosure was significantly enhanced when administered in conjunction with autophagy inhibitors. Autophagy inhibition triggers significant NK cell chemotaxis alongside upregulated CCL5 and CXCL10 gradients, while at the same time decreasing amounts of tumor-promoting CCL2 and CXCL12. In other words, the release of these chemokines attracts the engineered NK cell to the cancer TME, efficiently bring them where they are needed to fight the disease.

Furthermore, disabling autophagy also revealed sophisticated and complex reorganization of anti-GBM immunological responses that beneficially contributed to enhanced NK effector function. Clinical efficacy of adding autophagy inhibitors to GBM therapy was demonstrated in a prospective controlled randomized trial with the chronic administration of chloroquine (CQ), a common, FDA-approved autophagy inhibitor. The results demonstrated a significantly enhanced response of GBM to antineoplastic therapy and sensitized the cancer cells to exogenous agents, indicating that such treatments are clinically safe and well-tolerated.

Accordingly, in at least one exemplary embodiment, pharmacologic impairment of the autophagic process may be achieved through administration of a therapeutic treatment comprising an autophagy inhibitor in conjunction with, in series with, or before or after administration of a composition (or active ingredient) comprising engineered cells expressing the constructs of the present disclosure. Such autophagy inhibitor may comprise any therapy that is capable of inhibiting or reducing autophagy in the subject including, for example, administration of a small molecule inhibitor. In at least one embodiment, the small molecule inhibitor may comprise CQ, hydoroxycholorquine, saputin-1, SAR405, vertoprofin, or any other small molecule inhibitor now known or hereinafter developed that is suitable for administration to a patient to inhibit or block autophagy. Additionally or alternatively, autophagy may be genetically inhibited through the downregulation of one or more genes associated with autophagy, for example, through gene knockdown techniques. In at least one exemplary embodiment, the gene may comprise one or more of the following genes BECN1, p62, β-actin, ATG5, ATG7, LC3B, ATG12, ATG16L1 PM3K-III, ULK1, ULK2, FIP200, and LAMP2, or any other gene associated with (and capable of inhibiting via downregulation or otherwise) the autophagy pathway.

Whether the composition itself comprises a combination of active ingredients or it is delivered alone or in combination with other agents or therapies, the pharmaceutical compositions hereof can be delivered via various routes and to various sites in a mammal, preferably a human, body to achieve a particular effect. One skilled in the art will recognize that, although more than one route can be used for administration, a particular route can provide a more immediate and/or more effective reaction than other routes. For example, intratumoral delivery may be used for the treatment of a solid tumor cancer (and may be advantageous in terms of minimizing off-target effects). Local or systemic delivery can be accomplished by administering the pharmaceutical composition into body cavities, infusion, or by parenteral introduction. When the compositions of the present disclosure are administered in addition to an additional therapeutic treatment such as autophagy inhibition, the autophagy inhibition may be performed via systemic injection or infusion, or as otherwise desired by the healthcare provider.

The pharmaceutical compositions may be formulated in a variety of forms adapted to a preferred route of administration. Accordingly, a composition can be administered via known routes including, without limitation, parenteral (e.g., intradermal, subcutaneous, intravenous, transcutaneous, intramuscular, intraperitoneal, etc.) or topical (e.g., intratracheal, intrapulmonary, etc.). A composition can also be administered via a sustained or delayed release.

A formulation may be conveniently presented in unit dosage form and may be prepared by methods well known in the art of pharmacy. Methods of preparing a composition with a pharmaceutically acceptable carrier include the step of bringing NK cells (and/or stem cells or iPSCs) modified to express a construct of the present disclosure into association with a carrier that constitutes one or more accessory ingredients. In general, a formulation may be prepared by uniformly and/or intimately bringing the engineered cells into association with, for example, a liquid carrier.

A pharmaceutical composition that includes NK cells (and/or stem cells or iPSCs) modified to express a construct hereof may be provided in any suitable form including but not limited to a solution, a suspension, an emulsion, a spray, an aerosol, or any form of mixture. The composition may be delivered in formulation with any pharmaceutically acceptable excipient, carrier, or vehicle. The effective amount of NK cells (and/or stem cells or iPSCs) modified to express a construct hereof that is administered to a subject can vary depending on various dosing factors discussed herein.

In some embodiments, the method can include administering a therapeutically effective amount of engineered cells modified to express a construct of the present disclosure to provide a dose of, for example, at or greater than about 10 cells/subject, or from about 10 cells/kg to about 10¹⁰ cells/kg to the subject, although in some embodiments the methods may be performed by administering an amount of engineered cells in a dose outside these ranges.

In some embodiments, the pharmaceutical composition that includes engineered cells modified to express a construct hereof may be administered, for example, from a single dose to multiple doses per week, although in some embodiments the method can be performed by administering the pharmaceutical composition at a frequency outside this range.

In any event, the amount of engineered cells administered should take into account the route of administration and should be such that a sufficient number of the engineered cells will be introduced so as to achieve the desired therapeutic response. Generally, the pharmaceutical composition is administered to a subject in an amount, and in a dosing regimen effective to treat the symptoms or clinical signs of the condition, which may include (without limitation) reducing, limiting the progression of, ameliorating, or resolving the same (to any extent).

The constructs, engineered cells and NK cell lines of the present disclosure may be used in many applications including, without limitation, treating a subject having an adenosine overexpressing cancer or other disease state through reducing the size of a tumor or other targeted cell or preventing the growth or re-growth of a tumor or other cancerous or malignant cells in treated subjects. Accordingly, embodiments of a method for treating a subject having an adenosine overexpressing cancer or related disease state are also provided.

Methods of treating a subject suffering from a cancer using immunotherapy treatment are also provided. Such a method may comprise a step of administering (or having administered) to a subject a therapeutically effective amount of a pharmaceutical composition as described herein. For example, the pharmaceutical composition may comprise a population of engineered NK cells (as described herein) that express a polynucleotide construct encoding at least a first binding domain or fragment thereof that targets at least one cognate ligand on a target cell (e.g., NKG2D), at least a second binding domain or fragment thereof that is specific for GD2 (e.g., anti-GD2 and, optionally, scFv), and at least a third binding domain or fragment thereof that is specific for an adenosine-producing or an adenosine-intermediary producing cell surface protein of a target cell (e.g., anti-CD73 and, optionally, scFv). As described herein, the signaling domains may also be encoded which comprise one or more domains involved in promoting cytotoxic or cytolytic activity of the engineered cell upon activation by the associated binding domain binding the target cell. The target cell may comprise a a cancer cell (e.g., GBM) or a malignant cell in a TME, for example.

The administration step may be performed using any of the administration techniques heretofore described including, without limitation, intravenously, intratumorally (locally), parenterally, or via infusion (systematically). Furthermore, the pharmaceutical compositions hereof comprising engineered cells modified to express one or more of the constructs of the present disclosure may be assembled into a kit for treating a subject experiencing an adenosine overexpressing cancer, such as glioblastoma. It will be appreciated that, in conjunction with the novel pharmaceutical compositions of the presents disclosure, such kits may further include one or more tools and/or devices desirable to simplify and/or facilitate administration of such compositions to the subject (e.g., vials, syringes, tubing, etc.).

In at least one exemplary embodiment, the methods of treating a subject suffering from a cancer may further comprise the step of administering (or having administered) an additional therapeutic treatment to the subject comprising one or more autophagy inhibitors as described herein. For example, such autophagy inhibitor may comprise a therapeutically effective amount of a small molecule inhibitor and/or the genetic downregulation of a gene in the autophagy pathway. In at least one exemplary embodiment, the method comprises administering (or having administered) chloroquine to the subject at a concentration of between 0.01 μM and 200 μM. Administration of the additional therapeutic treatment may be performed as appropriate, including without limitation, via systemic injection or infusion.

Where the method comprises at least a dual approach (i.e. administration of the pharmaceutical compositions of the present disclosure and the additional therapeutic treatment comprising an autophagy inhibitor), the means of administering both treatment modalities need not be the same or occur at the same time or via the same dosage patterns. For example, the pharmaceutical composition may be administered intravenously, intratumorally, parenterally, or via infusion, and administration of the additional therapeutic treatment is performed via systemic injection or infusion. As such, the timing and dosages of the treatments may be tailored to the specific patient and his or her condition.

Furthermore, kits may also be provided to facilitate the multiple treatment approaches hereof. In at least one exemplary embodiment, the kit may comprise a therapeutically effective amount of a pharmaceutical composition according to the present disclosure, and a therapeutically effective amount of an autophagy inhibitor. In at least one embodiment, such treatment components are housed separately within the kit. It will be appreciated that, in conjunction with the such treatment compositions, the kits may further include one or more tools and/or devices desirable to simplify and/or facilitate administration of such compositions to the subject (e.g., vials, syringes, tubing, etc.).

Optionally, the method may also comprise steps of preparing the pharmaceutical composition for the subject. For example, optional step may comprise withdrawing, or having withdrawn, a sample, such sample comprising stem cells, blood cells, or iPSCs. Such withdrawn cells are thereafter isolated from the sample (i.e. in the case of a sample comprising a peripheral blood draw, one or more NK cells are isolated) and, if needed or desired, expanded. The sample may be obtained from the subject (e.g., an autologous cancer immunotherapy) and adoptive cell therapy is performed therewith. Alternatively, the sample may be provided from a donor separate from the subject (e.g., an allogeneic therapy). In at least one embodiment, the isolation, genetic modification, and/or any expansion steps are performed in vitro.

The method may also comprise optional step comprising transducing or transfecting the isolated cells are with an expression vector containing a construct of the present disclosure. Thereafter, a population of engineered cells are achieved that express the desired construct. Such population of engineered cells may then be administered to the subject as previously described. In at least one embodiment, such administration comprises adoptive cell therapy.

Furthermore, it is contemplated that the method may be combined with (or include) the administration of additional therapies now known or hereafter developed for the treatment of cancer, solid tumors, and/or related to ameliorating or eliminating symptoms or side-effects associated with such therapies. In at least one exemplary embodiment, the method may comprise the additional step of administering (or having administered) to a subject additional therapeutic treatment comprising an autophagy inhibitor as previously described.

In at least one embodiment of such a method, a construct of the present disclosure is introduced into an isolated NK cell of the subject and, thereafter, the transformed NK cell is reintroduced into the subject, thereby effecting anti-tumor and/or anti-cancer responses to reduce or eliminate the condition in the subject. Suitable NK cells that can be used are addressed above and include, without limitation, blood-derived NK cells. Even non-NK cells as set forth herein may be employed. As is well known to one of ordinary skill in the art, various methods are readily available for isolating these cells from a subject, such as leukapheresis.

While various embodiments of constructs, engineered cells and cell lines, pharmaceutical compositions, and methods hereof have been described in considerable detail, the embodiments are merely offered by way of non-limiting examples. Many variations and modifications of the embodiments described herein will be apparent to one of ordinary skill in the art in light of the disclosure. It will therefore be understood by those skilled in the art that various changes and modifications may be made, and equivalents may be substituted for elements thereof, without departing from the scope of the disclosure. Indeed, this disclosure is not intended to be exhaustive or too limiting. The scope of the disclosure is to he defined by the appended claims, and by their equivalents.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

### Pro-Tumorigenic Antigens and Construct Design Considerations

The antigen target selections of the present disclosure were based on the combinatorial presence of pro-tumorigenic antigens in GBM patient samples. It was observed that the gene level transcriptional expression of GD2, NKG2DL and CD73—markers with distinct roles contributing to GBM pathology—is present, individually, on over 96% (151 of 156) of the GBM RNA-seq patient dataset (156 patients) from The Cancer Genome Atlas (TCGA) patient cohort, while dual combinations were detected on 68% of patient samples. Patient-derived GBM cells—pediatric, adult primary, and adult recurrent—further confirmed this expression pattern.

Modulating the strength of antigen recognition to target antigens present in low amounts can enhance activation signals in settings of poor antigenic density, but it also potentiates off-target toxicities for those antigens that are present on normal cells. Targeting a combination of antigens that couples direct antigen recognition (for example, via CAR-based activation) with the sustained, spatially-controlled release of soluble antibody fragments subdues potential off-target effects and provides targeting combinations in patients for which these antigens show altered expression over time or in individual tumors. Accordingly, pursuant to the present disclosure, in at least one exemplary embodiment, NK cells were engineered to express a trifunctional construct that can target heterogeneous combinations of antigens responsively to their expression levels, locally and while sparing healthy cells. These multifunctionally-armed CAR-NK cells revealed no obvious toxicity towards to normal cells in the brain, including human cerebral microvascular endothelial cells (hCMEC/D3) and human cortical neuronal cells (HCN-2) (see Examples below). This is aided, at least in part, by the low expression of at least one of the targeted markers on these cells. The triggerable release of CD73 antibody fragments by GBM TAPs results in low concentrations of CD73 in the local TME, a sustained response that depends on CAR expression but is independent of its activation, providing two related but independent mechanisms of tumor recognition.

Several studies have demonstrated significant phenotypic similarity between blood-derived NK cells from both healthy donors and GBM patients. However, the phenotype and function of GBM tumor-resident NK cells are markedly altered and characterized by significantly lower levels of the activating receptors CD16, NKG2D, NKp30, NKp46, DNAM-1, CD2 and 2B4. Mechanistically, GBM patients utilize their own immunosuppressive TME to impair infiltrated NK cell function in favor of GBM escape from immune surveillance and NK-mediated cytotoxicity. These immune escape mechanisms are rooted in GBM's heterogeneity, and represent a complex network of pathways that promote GBM progression.

NKG2D-NKG2DL interactions play a vital role in activating the anticancer immune response. However, as previously discussed herein, NKG2D expression on the surface of NK cells is significantly decreased in response to the high extracellular concentrations of adenosine produced by the ectoenzyme CD73 in the GBM TME. In that context, the present inventors have previously established that CD73 is a significant prognostic biomarker for GBM and can be used as correlative factor of GBM patient survival. Adding to this, the present disclosure achieves compositions, cells, and methods that can induce upregulation of NKG2D through the use of inventive engineered NK cells that express an NKG2D-based CAR, which has been previously shown to enhance anti-tumor responses in combination with CD73 blockade. Immunosuppression of NK cells via the adenosinergic axis goes beyond activating receptor inhibition, however, and encompasses NK cell metabolism and various effector functions. Because of the wide expression of CD73, localizing targeted blockade of this enzyme may be preferential to systemic administration of anti-CD73 therapies.

To achieve controlled and localized responsiveness, NK cells were engineered to possess a tumor-responsive, locally-released anti-CD73 scFv that, upon triggering for release in the GBM TME, is capable of blocking local CD73 activity. The data presented herein supports that the engineered NK cells of the present disclosure not only reduce the local concentration of extracellular adenosine, but also reduce the immunosuppression of NK cell activation in a tumor-specific manner. The sustained, low concentration release of anti-CD73 sustains the metabolic function of NK cells that is otherwise lost in a setting of unencumbered CD73 enzymatic activity (i.e. the TME). In that respect, CAR-mediated signaling in combination with local CD73 blockade results in anti-tumor responses that are not only measurable by cytotoxicity against cancer cells, but also by their impaired metabolic activity, in turn sustaining the longer-term retention of NK cells in the tumor.

Temporal modulation of antigens also occurs in response to treatment. The expression of NKG2DLs, which include two MHC class I chain-related proteins (MICA and MICB) and six UL16-binding proteins (ULBP1-6), is upregulated in GBM following standard treatment with chemotherapy (TMZ) or irradiation (IR). As supported by data provided in the Examples below, NKG2DL expression may also be regulated by an autophagy inhibitor and treatment therewith can induce a significant increase in NKG2DL (both at MFI and % level) on patient-derived GBM cells. NKG2DL expression was previously found to inversely correlate with GBM cell maturity, with stem-like GBM cells expressing a more substantial, though heterogeneous, pattern of expression of these ligands. The changes induced by autophagy inhibitors sensitizes GBM to effector function via the NKG2DL-R axis and also promotes the indirect loss of NKG2D on effector cells through a phenomenon that can be lessened by the induction of NKG2D expression via CARs. Further, the reorganization of the GBM TME induced by autophagy inhibitors includes a decrease in GD2 expression, which lessens the apoptotic burden imposed on tumor-infiltrating cells induced by gangliosides on GBM. To recapitulate some of the stem cell-like properties associated with resistance to therapy due to glioma stem-like cells in patient GBM tumors, patient-derived xenografts were utilized that were generated with cells sourced from patients with primary GBM.

Antigenic targeting may not be sufficient to induce sustained anti-GBM responses. Clinical data have indicated that intratumoral NK cell presence associates positively with patient outcome and survival. However, NK cells are present in low amounts in GBM. Among regulating a variety of pathophysiological functions in GBM, chemokine receptor/ligand interactions can drive NK cell trafficking alongside their signaling gradient to result in improved homing, responses that were demonstrated for the CXCR4/CXCL12-directed NK cell homing to GBM. However, scant evidence of chemokine-dependent trafficking has been shown in intracranial GBM. The present disclosure reveals a significant relevance of the CCL5 and CXCL10 chemokine pathways in GBM upon pharmacological targeting of autophagy. Mechanistically, it was determined that inhibition of autophagy in GBM cells triggers the upregulation of chemokines, particularly CCL5 and CXCL10, via the activation of the PI3K/NF-κB signaling pathway. This subsequently conferred significant chemotactic ability to NK cells resulting in pronounced detection of adoptively-transferred CD73.mCAR-pNK cells in the brains of treated mice. Although significant upregulation of these chemokines was detected, contributions from others to the intratumoral trafficking of NK cells cannot be definitively ruled out at this time. Conversely, expression of CCL2 and CXCL12, both recognized for their contributing roles to the recruitment of immunosuppressive cells such as tumor associated macrophages (TAMs), was significantly decreased in response to autophagy blockade. The preclinical in vivo data presented herein further demonstrates that autophagy inhibitor-mediated targeting of autophagy potentiates anti-GBM activity of NK cells via mechanisms that may involve a sensitization to their killing by modulating antigen level expression, and the activation of pro-apoptotic pathways on GBM. These data point to novel actionable responses induced by the inhibition of autophagy and CAR-based antigenic targeting. The data presented herein also highlight the complex and sophisticated reorganization of the GBM TME induced by administration of an autophagy inhibitor. Confounding factors of the dichotomous effects of an autophagy inhibitor on GBM pathology have been recognized clinically. As an adjuvant, CQ (a representative example of an autophagy inhibitor) was shown to enhance clinical responses to anti-GBM therapy in double-blinded Phase III studies. The data supports that CQ was able to cross the blood-brain barrier in vivo, while preclinical studies have demonstrated that concentrations of 50 mg/kg are suitable for administration regimens against orthotopic glioblastoma xenografts.

The multiple effects of autophagy inhibitors on the phenotypic signatures and chemokine profiles of GBM in vivo, alongside a limited ability to control tumor burden alone, however, point to a nuanced role in combination regimens which can nonetheless uniquely enhance CAR-based adoptive transfer immunotherapy. It is ideal that the potential toxicity of many autophagy inhibitors such as CQ should be factored into the therapy administration regimens. The use of the less toxic metabolite hydroxychloroquine is one strategy to mitigate toxicity associated with CQ in particular. Here, in cases where mice were treated with a combination of CQ and NK cells, NK cells were infused intracranially, while CQ was administered systemically. Significantly, no observable CQ-dependent toxicity in the treated groups was observed.

The present disclosure and related data demonstrates that development of multifunctional genetically-engineered human NK (CD73.mCAR-pNK) cells can result in effective anti-GBM activity supported by a concerted approach of overcoming tumor heterogeneity and multiple immunosuppressive features of the GBM TME. The present disclosure also uncovers that targeting autophagy functions as an immuno-modulator to promote the homing of effector CAR-NK cells into GBM tumor sites while reprogramming the GBM TME toward sensitization to CAR-based targeting. Indeed, all treated mice experienced either a delay or complete arrest of tumor growth with the most significant responses obtained upon co-administration of CAR-NK cells with an autophagy inhibitor. As a demonstration of the use of human CAR-NK cells in immunotherapy of GBM, the data shows that, while human CAR-NK therapy is a viable option, in certain embodiments, optimal responses may rely on administration regiment, dosage and frequency of adoptive transfer.

### EXAMPLES

The following examples illustrate certain specific embodiments of the present disclosure and are not meant to limit the scope of the invention in any way.

### Mice

Female 6- to 8-week-old Rag1^{-/-} mice and NOD.Cg-Prkd^{scid} IL^{rgtm1Wj1}/SzJ (NSG) mice were maintained at the Purdue Center for Cancer Research. All animal experiments described herein were approved by the Purdue University Animal Care and Use Committee.

### Isolation of Peripheral Blood NK Cells and Cell Culture

Blood samples were obtained from healthy adult donors and pNK cells were isolated from whole blood by negative selection using the EasySep™ Direct Human NK cell Isolation Kit (StemCell Technologies, Canada). The cells were then cultured and expanded using NK MACS® medium system according to the manufacturer's instructions (130-114-429; Miltenyi Biotec, Germany). NK-92 cells (directly purchased from the American Type Culture Collection (ATCC) were maintained in RPM11640 supplemented with 10% FBS, 100 U/mL penicillin, 100 μg/mL streptomycin, 2 mM L-glutamine, 400 U/mL IL-2 and 0.1 mM 2-mercaptoethanol. HCN-2 cells (directly purchased from ATCC) were maintained in DMEM supplemented with 10% FBS, 100 U/mL penicillin, and 100 μg/mL streptomycin. hCMEC/D3 cells (Purdue University, Indiana) were grown in EBM-2 supplemented with 5% FBS, 100 U/mL penicillin, 100 μg/mL streptomycin, 1.4 μM hydrocortisone, 5 μg/mL ascorbic acid, 1% chemically defined lipid concentrate, 10 mM HEPES and 1 ng/mL bFGF. SJ-GBM2, GBM43, and GBM10 cells (Indiana University School of Medicine, Indiana) were grown in DMEM supplemented with 10% FBS and 1% HEPES. All cell lines were incubated at 37° C. in a humidified 5% CO₂ environment.

### Chemical Reagents

Chloroquine diphosphate salt (CQ) (98%) and adenosine 5′-monophosphate sodium salt hydrate (AMP) (99%) were purchased from ACROS Organics™ (Fair Lawn, N.J.). Hydrocortisone (98%) was purchased from Alfa Aesar (Haverhill, Mass.). Ascorbic acid, sodium chloride (NaCl) and bovine serum albumin (BSA) were purchased from Thermo Fisher Scientific (Waltham, Mass.). Potassium chloride (KCl), magnesium chloride (MgCl₂), sodium bicarbonate (NaHCO₃), adenosine (>99%), DEAE-dextran hydrochloride and glucose were purchased from Sigma (St. Louis, Mo.). Brefeldin A Solution 1000× and Monensin Solution 1000× were purchased from BioLegend (San Diego, Calif.). D-luciferin potassium salt (>99%) was purchased from Syd Labs (Natick, Mass.). LY294002, BAY11-782 and SP600125 were purchased from Cayman Chemical (Ann Arbor, Mich.). Fetal bovine serum (FBS) and dimethyl sulfoxide (DMSO) were purchased from Corning (Corning, New York). Recombinant human interleukin-12 (IL-2) was gifted from Akron Biotech (Boca Raton, Fla.). Recombinant human interleukin-15 (IL-15) and fibroblast growth factor-basic (bFGF) were purchased from GoldBio (St. Louis, Mo.). Recombinant human RANTES (CCL5) was purchased from PeproTech (Rocky Hill, N.J.). Recombinant human CXCL10, recombinant human u-plasminogen activator/urokinase, CF (uPA) and recombinant human NKG2D/CD314 Fc chimera were purchased from Research and Diagnostic Systems, Inc. (Minneapolis, Minn.). Biotin-protein L was purchased from GenScript Biotech (Piscataway, N.J.). BsiWI was purchased from New England Biolabs (Ipswich, Mass.). RPMI1640, DMEM, IMDM, penicillin/streptomycin solution 100×(PS), 2-mercaptoethanol (50 mM), HEPES (1 M), chemically defined lipid concentrate, trypan blue solution and trypsin-EDTA were purchased from Gibco™, Thermo Fisher Scientific (Waltham, Mass.). Opti-MEM Reduced Serum Media was purchased from Invitrogen (Carlsbad, Calif.). EBM-2 was purchased from Lonza Group AG (Basel, Switzerland). Human AB serum was purchased from Valley Biomedical (Winchester, Va.). Collagen I, rat tail was purchased from Enzo Life Sciences, Inc. (Farmingdale, New York). RIPA lysis buffer system (sc-24948) was purchased from Santa Cruz Biotechnology, Inc. (Dallas, Tex.).

### Plasmid Construction and Lentvirus Production

(1) (2) (3) and (4) below were custom-cloned and produced by vectorbuilder.com. (5) was purchased from Santa Cruz Biotechnology, Inc. (Dallas, Tex.). (6) was purchased from GenTarget Inc. (San Diego, Calif.).
(1) pT7[mRNA]-NKG2D-CAR: In this plasmid, a human NKG2D-specific CAR (NKG2D-DAP10-CD3ζ; identified as Construct 1B below) was expressed under the control of the T7 promoter. The NKG2D sequence was derived from previous work.
(2) pT7[mRNA]-GD2-CAR: In this plasmid, a human GD2-specific CAR (anti-GD2 scFv-CD8 Hinge-hCD28-CD3ζ; identified as Constrict 1A below) was expressed under the control of the T7 promoter. The anti-GD2 scFv sequence was derived from previous work.
(3) pT7[mRNA]-CD73-CAR: In this plasmid, the entire construct, including a human CD73-specific cleavable anti-CD73 scFv, the human GD2-specific CAR (GD2 scFv-CD8 Hinge-hCD28-CD3ζ; Constrict 1A) and the human NKG2D-specific CAR (NKG2D-DAP10-CD3ζ; Construct 1B) were expressed under the control of the T7 promoter. Here, the anti-CD73 scFv fragment was coupled with GD2-CAR through a linker comprising SEQ ID NO: 1, a cleavable peptide fragment (SEQ ID NO: 2) and a short spacer (GSSGT). The GD2-CAR was associated with NKG2D-CAR through “self-cleaving” P2A peptides (SEQ ID NO: 4). The anti-CD73 scFv sequence was derived from previous work.
(4) pLV[Exp]-Puro-EF1A>{CD73-CAR}: The lentiviral vector encoding the entire construct (Construct 1), described in (3) was expressed under the control of the EF1alpha promoter.
(5) Beclin 1 (BECN1) shRNA (h) Lentiviral Particles (sc-29797-V): BECN1 gene knockdown BECN1⁻GBM43 cells were generated by lentiviral transduction according to the manufacturer's protocol.
(6) CMV-Luciferase (firefly), (Puro) Lentiviral Particles (LVP325): The firefly luciferase-labeled GBM43: GBM43 (Luc) cells were generated by lentiviral transduction with a luciferase-bearing lentiviral vector according to the manufacturer's protocol.

### Materials

Table 1 lists the antibodies and stains used in the experiments described herein and assay kits and primers described in the present disclosure are listed below.

TABLE 1
Antibodies and Stains

| Name | Clone | Company |
|---|---|---|
| APC anti-human NKG2D/CD314 | 1D11 (RUO) | BD Biosciences (Franklin |
| | | Lakes, NJ) |
| FITC anti-human CD56 | HCD56 | BioLegend (San Diego, CA) |
| PE-Cy7 anti-human CD3 | HIT3 | BioLegend (San Diego, CA) |
| APC anti-human CD107a | H4A3 | BioLegend (San Diego, CA) |
| APC anti-human IFN-γ | 4S.B3 | BioLegend (San Diego, CA) |
| APC Mouse IgG1, κ Isotype Ctrl | MOPC-21 | BioLegend (San Diego, CA) |
| APC anti-human GD2 | 14G2a | BioLegend (San Diego, CA) |
| APC Mouse IgG2a, κ Isotype Ctrl | MOPC-173 | BioLegend (San Diego, CA) |
| APC anti-human CD73 | AD2 (RUO) | BD Biosciences (Franklin |
| | | Lakes, NJ) |
| APC anti-human IgG Fc | M1310G05 | BioLegend (San Diego, CA) |
| APC anti-human CD16 | B73.1 | BioLegend (San Diego, CA) |
| APC anti-human NKG2A | #131411 | Research & Diagnostic |
| | | Systems, Inc. |
| | | (Minneapolis, MN) |
| APC Mouse IgG2b, κ Isotype Ctrl | MPC-11 | BioLegend (San Diego, CA) |
| PE anti-human HLA-A, B, C | W6/32 | BioLegend (San Diego, CA) |
| PE Mouse IgG2a, κ Isotype Ctrl | MOPC-173 | BioLegend (San Diego, CA) |
| APC Streptavidin | | BioLegend (San Diego, CA) |
| Human NKp46/NCR1 Antibody | | Abcam (Cambridge, UK) |
| Mouse NKp46/NCR1 Antibody | | Research & Diagnostic |
| | | Systems, Inc. |
| | | (Minneapolis, MN) |
| NT5E/CD73 (D7F9A) Rabbit mAb | | Cell Signaling Technology |
| | | (Danvers, MA) |
| Human CCL5/RANTES Antibody | | Research & Diagnostic |
| | | Systems, Inc. |
| | | (Minneapolis, MN) |
| Human CXCL10 Antibody | | Research & Diagnostic |
| | | Systems, Inc. |
| | | (Minneapolis, MN) |
| SYTOX ™ Green Dead Cell Stain | | Invitrogen (Carlsbad, CA) |
| SYTOX ™ Blue Dead Cell Stain | | Invitrogen (Carlsbad, CA) |

The following assay kits were used in the examples described herein:

### (1) TransIT®-mRNA Transfection Kit (Mirus Bio LLC, Madison, Wis.);

(2) EasySep™ Direct Human NK cell Isolation Kit (StemCell Technologies, Vancouver, Canada);

### (3) Human CCL5 (RANTES) Biolegend-ELISA MAX™ Deluxe Sets (BioLegend, Sand Diego, Calif.);

### (4) Human CXCL10 (IP10) Biolegend-ELISA MAX™ Deluxe Sets (BioLegend, Sand Diego, Calif.);

### (5) Malachite Green Phosphate Assay Kit (BioAssay Systems, Hayward, Calif.);

### (6) Adenosine Assay Kit (Cell Biolabs Inc., San Diego, Calif.);

(7) mirVana™ miRNA Isolation Kit (Life Technologies, Carlsbad, Calif.);
(8) HiScribe™ T7 ARCA mRNA Kit (with tailing) (New England Biolabs, Ipswich, Mass.);

### (9) EZ-10 Spin Column RNA Cleanup & Concentration Kit (Bio Basic Inc., Ontario, Canada);

(10) qScript™ One-Step SYBR® Green qRT-PCR Kit, Low ROX™ (Quanta BioSciences Inc., Gaithersburg, Md.);

### (11) Fixation/Permeabilization Solution Kit (BDBiosciences, San Jose, Calif.);

### (12) 7-AAD/CFSE Cell-Mediated Cytotoxicity Assay Kit (Cayman Chemical, Ann Arbor, Mich.).

The following RT-PCR primers were used in the examples described herein: Human CCL5 qPCR primer pair (HP100784), Human CXCL10 qPCR pair (HP100690), Human CCL2 qPCR primer pair (HP104854), Human CXCL9 qPCR primer pair (HP100773) and Human CXCL12 qPCR primer pair (HP100192) were obtained from Sino Biological US Inc. (Chesterbrook, Pa.). GAPDH was used as the endogenous control. Its primers—primer 1 (SEQ ID NO: 4) and primer 2 (SEQ ID NO: 5)—were obtained from Integrated DNA Technologies, Inc. (Coralville, Iowa). All of the primers used in the RT-PCR assay were used according to the manufacturer's instructions.

### Statistical Analyses

Data is presented herein as mean±SEM. Statistical analysis for all studies was performed using Excel 2007 software (Microsoft Office 2007), and comparison between two normally distributed test groups was performed using the two-tailed Student's t-test. For analysis of three or more groups, comparison was performed using a one-way ANOVA analysis. p<0.05 was considered to be statistically significant.

### Example 1

### NK Cells Engineered with a Multifunctional, Responsive CAR-Based Construct can Target Multiple Antigens Widely Present in Human GBM

To identify any correlation between the expression profiles of genes corresponding to NK cells and a series of functional genes that encode various pro- or anti-tumorigenic ligands and targets in GBM, a gene-set scoring method was used to investigate a GBM RNA-seq patient dataset (156 patients) from The Cancer Genome Atlas (TCGA) through the Genomic Data Commons and relationships were identified between expression of certain functional genes and NK cell presence. Such functional genes included, without limitation, the NK cell signature gene set (including, for example, NCR1, NCR3, KLRB1, CD160, and PRF1), NT5E (which encodes CD73), B4GALNT1 (which encodes the enzyme that produces GD2), MICA B (which encodes common ligands for NKG2D), and CCL5 and CXCL10 (which encode two eponymous chemokines).

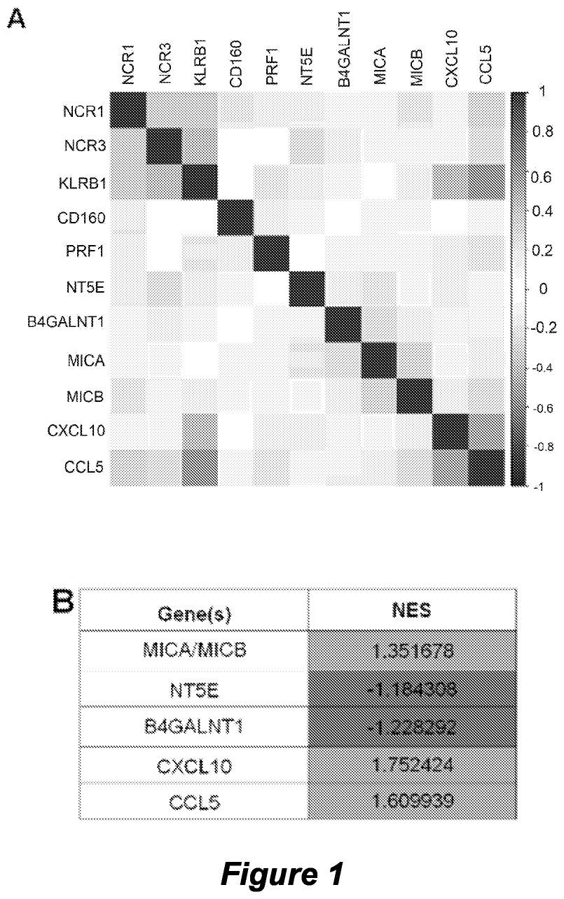

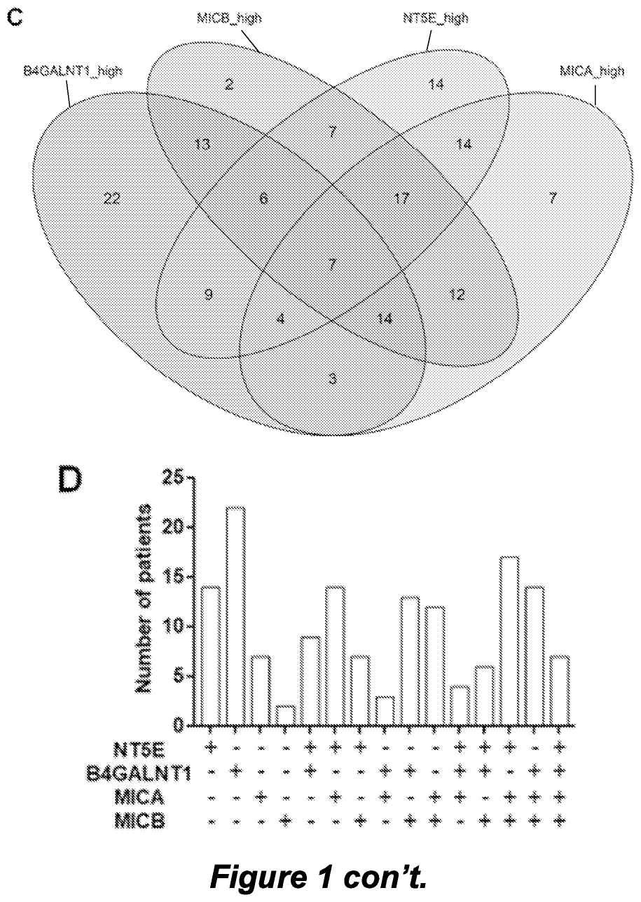

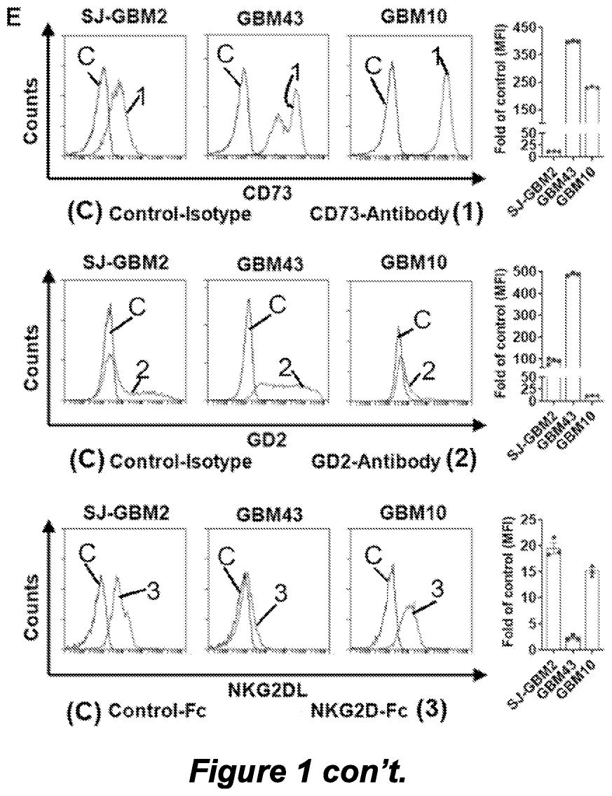

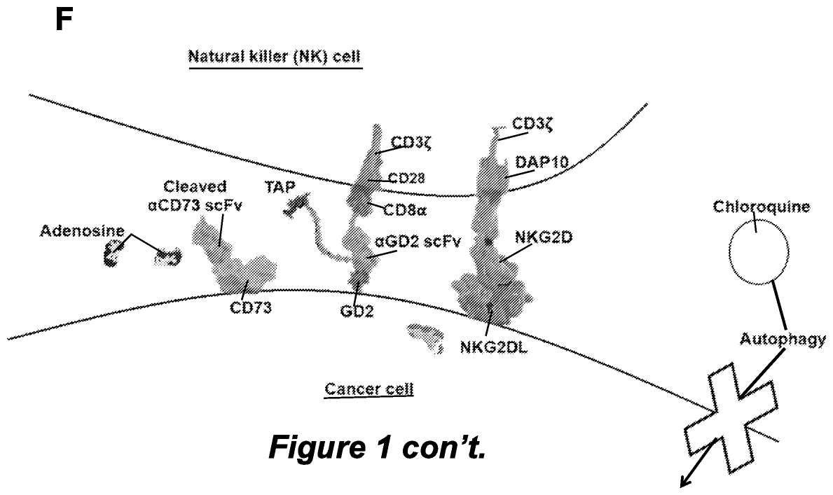

Linear correlation between normalized expression (FPKM) of selected genes was determined using corrplot R-package. The GBM Patients (N=156) were classified into high/low groups based on expression of each individual gene (B4GALNT1, MICA, MICB and NT5E) using the upper and lower quartiles as cutoff for high and low expression, respectively. The high expression group was of interest and number of patients with high expression of at least one of the 4 genes was used to build a Venn diagram (see FIG. 1, subpart C).

NT5E and B4GALNT1 each had a negative correlation with individual genes that represented the NK signature set in GBM, suggesting that the antigens encoded by these genes act to suppress NK cell function and proliferation in GBM. Alternatively, genes that are known to drive NK effector responses, including the NKG2D-ligand encoding MICA/B, CCL5, and CXCL10, revealed a positive correlation with individual NK signature genes (r<0) (see FIG. 1, subpart A).

Classification of GBM patients into high/low groups based on 50% upper and lower quartiles (using TCGA RNA-seq data) further supported these findings, identifying that 151 out of 156 patients overexpressed at least one of the four targeted genes NT5E, B4GALNT1, MICA, or MICB). Enrichment analysis against the NK signature gene set using GBM patients' data with high expression of specific genes indicated a negative correlation of expression (NES<1) for genes NT5E AND B4GALNT1, and a positive correlative expression (NES>1) for genes MICA/MICB, CXCL10 and CCL5 (see FIG. 1, subpart B and Table 2).

TABLE 2
Correlation between normalized expression (FPKM) of the entire NK five-gene
set and individual genes MICA/MICB, NT5E, B4GALNT1, CXCL10 and CCL5 based
on TCGA GBM data (NES: enrichment score; ES: normalized enrichment score.

| Pathway | pval | padj | ES | NES | Direction |
|---|---|---|---|---|---|
| MICA/MICB | 0.1374046 | 0.1374046 | 0.7373083 | 1.351678 | Positive |
| NT5E | 0.26546 | 0.26546 | −0.6885178 | −1.184308 | Negative |
| B4GALNT1 | 0.2709677 | 0.2709677 | −0.6425565 | −1.228292 | Negative |
| CXCL10 | 0.0072115 | 0.0072115 | 0.9004993 | 1.752424 | Positive |
| CCL5 | 0.0275424 | 0.0275424 | 0.8680867 | 1.609939 | Positive |

A Venn diagram showing the association of gene expression among patient numbers indicates a corresponding and expected lower number of patients expressing multiple gene combinations (see FIGS. 1C-1D).

The two pro-tumorigenic markers GD2 and CD73 are widely and heterogeneously expressed on different types of GBM, including patient-derived primary adult (GBM43), pediatric (SJ-GBM2), and recurrent adult (GBM10) brain tumor cells (see FIG. 1, subpart E). Among these, the ectoenzyme CD73 was previously demonstrated to be a negative prognostic factor.

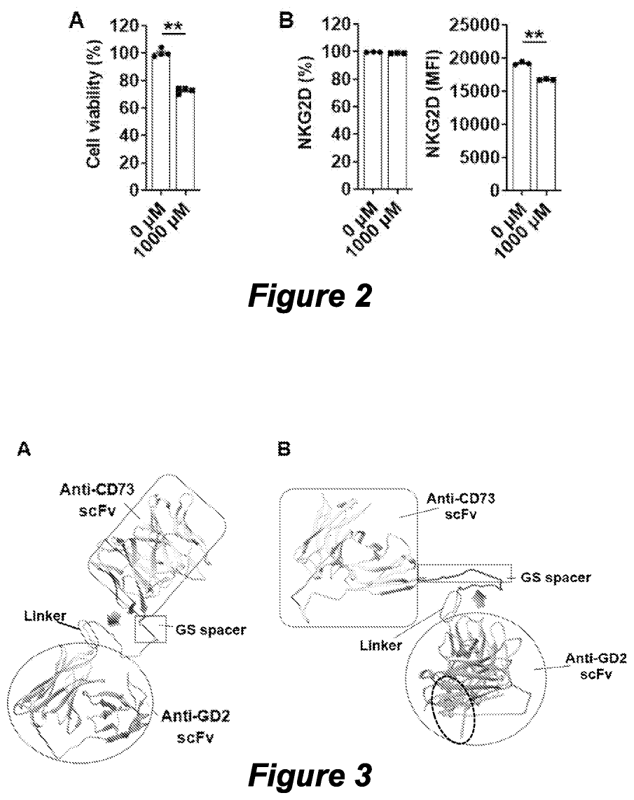

Conversely, cognate ligands to the potent activating NK cell receptor NKG2D were also highly present in GBM, though NKG2D is often downregulated in the TME. To verify this, pNK cells were seeded into a 24-well plate at a density of 5×10⁵ cells/well in 500 μL medium. Adenosine was then added to a final concentration of 1000 μM. After 24 h, the cells were collected and their viability (%) was determined via a CCK-8 assay (subpart A of FIG. 2). The NKG2D expression (% and MFI) was determined by flow cytometry (subpart B of FIG. 2). As shown in FIG. 2, the presence of adenosine had a clear negative affect on cell viability and similarly decreased the expression of NKG2D. These findings support that there is a need to increase NKG2D expression on NK cells through engineering.

To achieve responsive and combinatorial targeting of these GBM antigens, a bicistronic vector was first engineered to express two individual CARs simultaneously, yielding NK cells expressing both a GD2.CD28.CD3ζ CAR (Construct 1A) and NKG2D.DAP10.CD3ζ CAR (Construct 1B). Construct 1A was then further engineered in tandem, following a cleavable, tumor-sensitive linker, with an anti-CD73scFv to generate CD73-GD2.CD28.CD3ζ-CAR (Construct 1) (see FIGS. 3-4) and result in a construct configured for localized release of a CD73-blocking antibody fragment in the GBM TME independent of CAR activation.

Functionality of individual genetic components of the multifunctional Construct 1 was then evaluated by generating separate versions of each element, including Construct 1B (FIG. 5, subpart A), Construct 1A incorporating the cleavable anti-CD73 scFv (CD73-GD2.CD28.CD3ζ-CAR) (FIG. 5, subpart F) and the multifunctional CAR bearing the full construct (Construct 1; FIG. 5, subpart K).

Each CAR was separately expressed into NK-92 cells and expression of the corresponding CAR structure was verified (FIG. 5, subparts B, G and L). More specifically, mRNA individually derived from plasmid (1), (2) and (3) (referenced above) was synthesized through HiScribe™ T7 ARCA mRNA Kit (with tailing) (New England Biolabs, Ipswich, Mass.). All the mRNA products were concentrated and purified via EZ-10 Spin Column RNA Cleanup & Concentration Kit (Bio Basic Inc., Markham, Canada). After that, their concentrations were measured using a Qubit 4 Fluorometer (Thermo Fisher Scientific, Waltham, Mass.).

To generate the engineered NK-92 cells with the expression of different CAR structures, NK-92 cells were transfected with mRNA via TransIT®-mRNA Transfection Kit (Mirus Bio LLC, Madison, Wis.) according to the manufacturer's recommended protocols. Briefly, NK-92 cells were plated in 12-well plates (5×10⁵ cells/well in 1 mL complete growth medium). The mRNA (1 μg) was added into Opti-MEM (100 μL) in a sterile polystyrene tube and mixed well. Thereafter, the mRNA Boost Reagent (3 μL) and Trans Reagent (3 μL) were added and mixed sequentially. The tube was incubated at room temperature for 5 min, and the complexes were added dropwise to the cells. Following transfection, the cells were incubated at 37° C. and 5% CO₂ for 48 hours and then harvested for the CAR structures expression determination.

To measure the transfection efficiency via the levels of NKG2D expression (expression of Construct 1B and/or Construct 1), the cells were stained with APC-conjugated NKG2D antibody and analyzed by flow cytometry using a BD Accuri™ C6 Plus (Becton, Dickinson and Company, Franklin Lakes, N.J.).

For detection of GD2.CAR expression (expression of Construct 1A and/or Construct 1), the cells were sequentially stained with Biotin-Protein L and APC-streptavidin and the levels of GD2.CAR expression were analyzed by flow cytometry. For CD73.mCAR-NK-92 cells expressing the full CAR construct (Construct 1), expression of anti-CD73 scFv was determined by incubating the cells with various concentrations of uPA to trigger the removal of anti-CD73 scFv from the cell surface and the rest of the CAR-construct. Following this, the cells were stained with Biotin-Protein L and APC-streptavidin. The expression of the remaining construct was then analyzed by flow cytometry. The cell viability after transfection was measured by CCK-8 assay analysis (Dojindo Molecular Technologies, Inc., Rockville, Md.).

As shown in FIG. 5, subparts C, H, and M, in all cases the CAR-expressing NK cells retained high viability (>95%). At the same time, robust Construct 1B expression on NK cells was achieved (˜44%) (see FIG. 5, subpart D), and cells expressing Construct 1B showed higher cytotoxic activity of GBM43 cells compared to non-transfected cells (FIG. 5, subpart E). Similar expression was achieved with Construct 1A (˜42%) (see FIG. 5, subpart I), with a correspondingly higher cytotoxicity against patient-derived GBM target cells (see FIG. 5, subpart J). This data confirms that Construct 1A is capable of effectively recognizing and eliminating GD2-expressing GBM cells.

When engineered to express the complete multifunctional sequence (Construct 1), NK-92 cells showed a significant increase in NKG2D expression (FIG. 5, subpart N), anti-GD2 scFv expression, and anti-CD73 scFv expression (FIG. 5, subpart O).

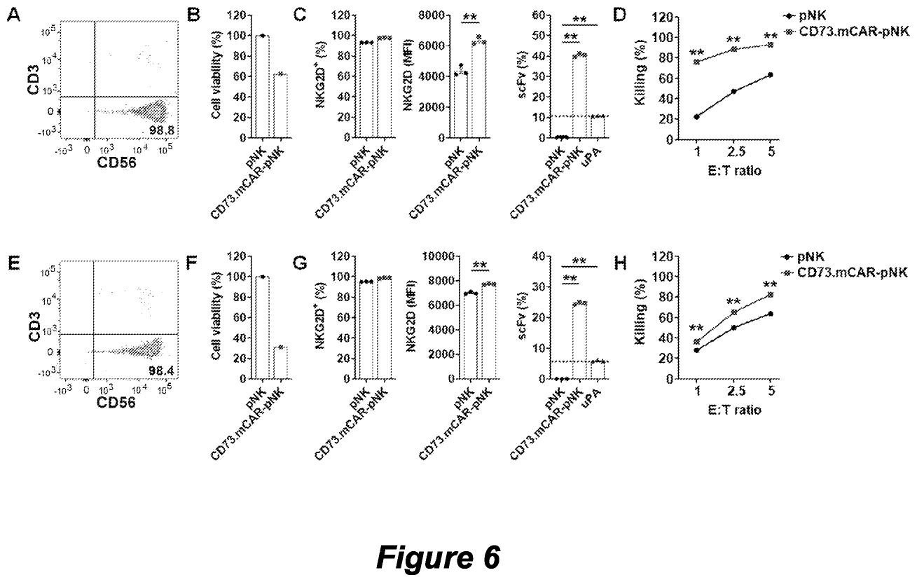

As compared to NK-92 cells, primary NK (pNK) cells derived from the peripheral blood of healthy donors generally possess higher lytic activity against patient-derived GBM target cells. To validate that patient-derived GBM43 cells can be killed by pNK cells collected from multiple donors and subsequently engineered to express Construct 1, pNK cells were isolated from healthy adult donors (FIG. 5, subpart P, and FIG. 6) and engineered by lentiviral transduction in the presence of DEAE-dextran to express the full multifunctional Construct 1 (CD73.mCAR-pNK).

Cytokine-activated pNK cells (activated for 1 week of culture in NK-MACS® medium) were engineered to express the functional CAR over two rounds of lentiviral transduction (pLVp[Exp]-Puro-EF1A>{CD73-CAR}) with the help of dextran. Briefly, pNK cells were plated in 24-well plates (5×10⁵ cells/well in 0.5 mL RPMI1640 medium supplemented with 10% FBS and 500 U/mL IL-2). The lentivirus supernatant was added at 10 multiplicity of infection (MOI) and, thereafter, the dextran aqueous solution was added to a final concentration of 8 μg/mL. Each plate was centrifuged at 1000 g for 60 min and incubated overnight in a 37° C. incubator infused with 5% CO₂. After transduction, the CD73.mCAR-pNK cells were harvested and expanded under the NK-MACS© medium until further use. The expression levels of all constructs were then measured and analyzed by flow cytometry according to the above-described protocol. The cell viability after transduction was measured by Trypan blue staining.

After two rounds of transduction, an expected, donor-specific drop in viability of the donor NK cells (FIG. 5, subpart 0, and FIG. 6) was accompanied with significantly increased expression of NKG2D, anti-GD2 scFv and anti-CD73 scFv (FIG. 5, subparts R and S, and FIG. 6). Moreover, the CD73.mCAR-pNK cells expressing Construct 1 were able to expand substantially over 2-weeks in commercial NK-MACS© medium without the use of feeders and while retaining stable gene expression (see FIG. 5, subpart T).

Collectively, these results validate the use of CD73, GD2 and NKG2DL as combinatorial GBM targets, and provide a rationale for investigating their use for NK cell-based GBM immunotherapy.

### Example 2

### Multifunctional Engineered NK Cells Target Patient-Derived GBM Cells while Sparing Normal Cells

To test for killing potency, a killing assay was utilized with either NK-92, NKG2D.CAR-NK92 (Construct 1B), GD2.CAR-NK92 (Construct 1A), CD73.mCAR-NK92 (Construct 1), pNK or CD73.mCAR-pNK cells were co-cultured with different target cells (SJ-GBM2, GBM43, GBM10, hCMEC/D3 and HCN-2) at various E:T ratios for 4 hours. To evaluate the effects of anti-CD73 scFv part on the killing activity, either CD73.mCAR-NK92 or CD73.mCAR-pNK cells with expression of the entire construct were incubated with uPA to remove all the anti-CD73 scFv from the surface. After that, the killing ability of these cells against GBM43 target cells was measured.

To test for degranulation, either CD73.mCAR-NK92 or CD73.mCAR-pNK cells were co-cultured with GBM43 cells (E:T=5:1) for 4 hours in the presence of APC-CD107a antibody and monensin pursuant to known techniques. Detection of CD107a (% and MFI) was carried out by flow cytometry.

To test for IFN-γ production, a known protocol was followed where either CD73.mCAR-NK92 or CD73.mCAR-pNK cells were co-cultured with GBM43 cells (E:T=5:1) for 4 hours in the presence of brefeldin A. The cells were then collected, washed, and fixed/permeabilized and subsequently stained with APC-IFN-γ antibody and detected by flow cytometry.

To test for CD73 activity and its ability to generate adenosine, GBM43 cells were seeded at 2×10⁴ cells per well in a 96-well plate in complete DMEM. After overnight incubation, GBM43 cells were incubated with anti-CD73 scFv following uPA (100 nM)-mediated cleavage from either CD73.mCAR-NK92 or CD73.mCAR-pNK cells during incubation for 6 hours. Then, the cell medium was removed and cells were rinsed three times with phosphate-free buffer (117 mM NaCl, 5.3 mM KCl, 1.8 mM MgCl₂, 26 mM NaHCO₃, 10 mM glucose, pH 7.4, diluted in ddH₂O). AMP (250 μM final), diluted in phosphate-free buffer, was added and incubated for 10 min at 37° C. Finally, the phosphate (Pi) concentrations resulting from AMP hydrolysis were measured using a malachite green phosphate assay kit following the manufacturer's instructions.

To test for changes in CD16 and NKG2A expression (i.e. NK cell phenotype changes) on NK cells in response to GBM cells, CFSE-labeled GBM43 cells were first seeded at 4×10⁴ cells per well in a 24-well plate in complete DMEM. After overnight incubation, either pNK or CD73.mCAR-pNK cells were added (E:T=5:1) for 4 hours. The cells were then collected, washed and stained with either APC-CD16 or APC-NKG2A antibody and the expression was measured by flow cytometry.

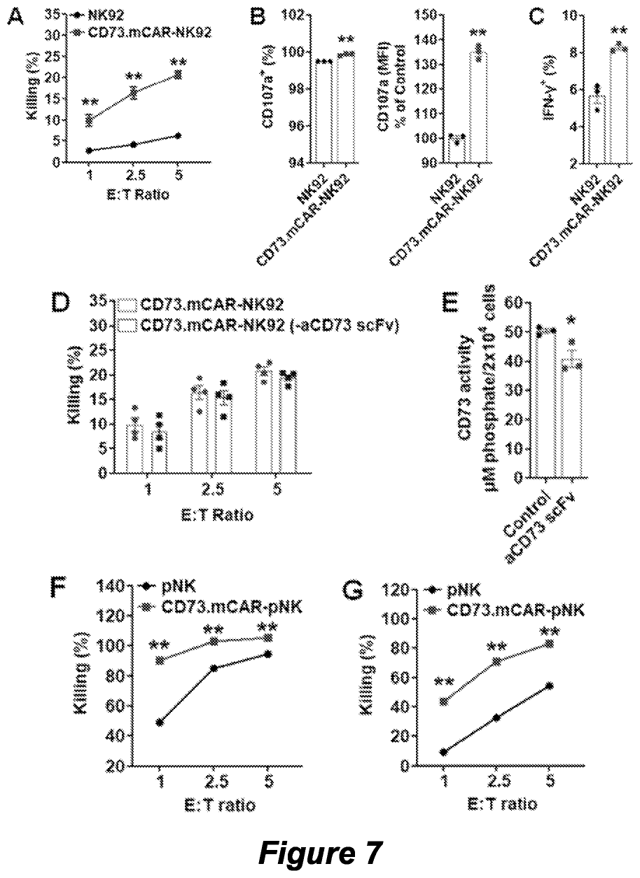

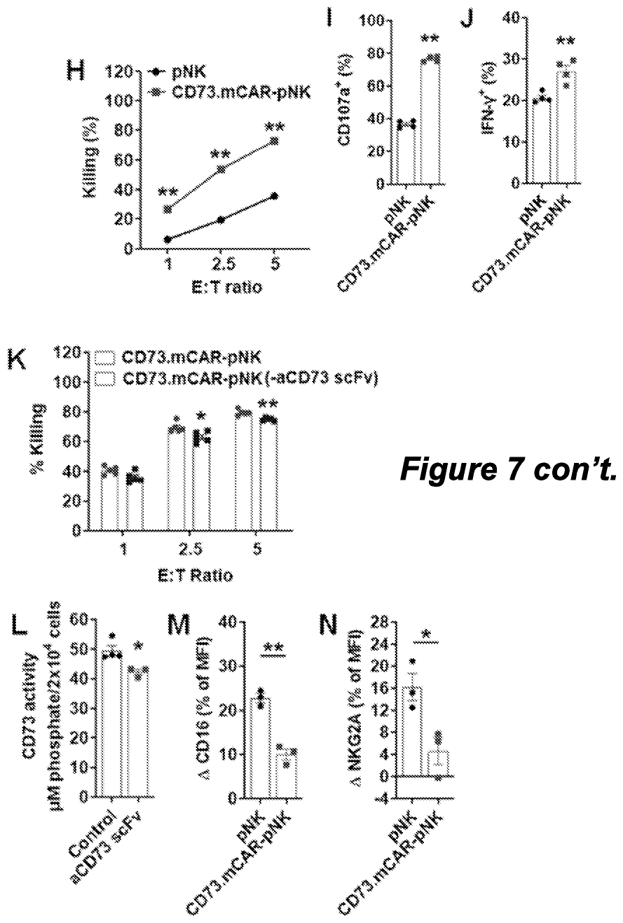

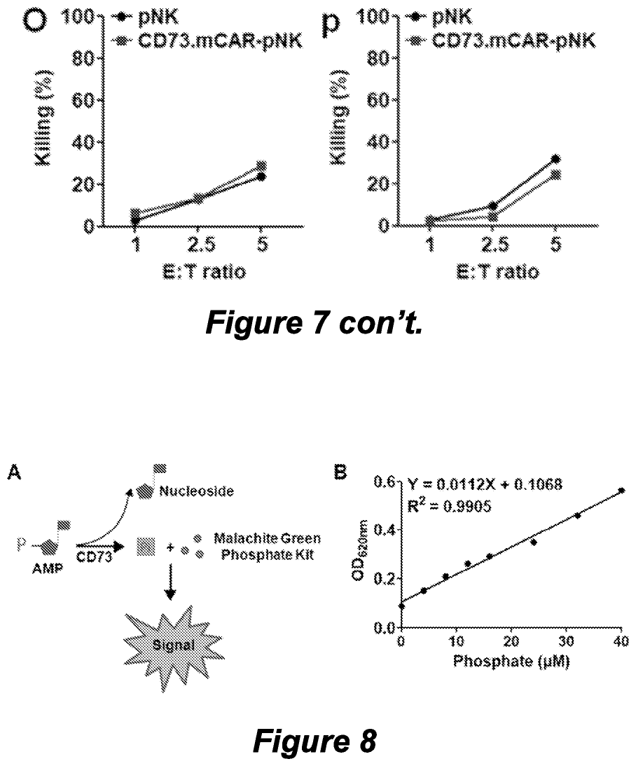

Compared to control NK-92 cells, CD73.mCAR-NK92 cells (expressing Construct 1) showed significantly improved killing of GBM43 cells after 4 h at various E/T ratios (FIG. 7, subpart A). Furthermore, co-culture with GBM43 cells stimulated CD73.mCAR-NK92 cells to induce degranulation as measured by cell surface CD107a expression (FIG. 7, subpart B) and up-regulate IFN-γ secretion (FIG. 7, subpart C).

After treating CD73.mCAR-NK92 cells with uPA (100 nM) to liberate the anti-CD73 scFv, the number of killed GBM43 cells decreased (FIG. 7, subpart D). Additionally, the cleaved anti-CD73 scFv resulted in significantly decreased production of extracellular adenosine by GBM43 cells (FIG. 7, subpart E, and FIG. 8), indicating that the enzymatic ability of CD73 on GBM was impaired. Additionally, this data confirms that the anti-CD73 scFv is functional and specific for CD73, and can abrogate adenosine accumulation.

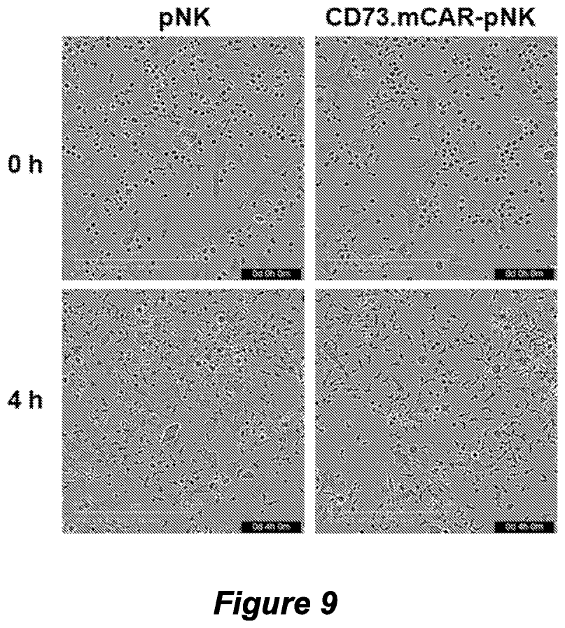

Additionally, the human peripheral blood-derived NK cells, genetically-engineered to express the multi-functional Construct 1 (CD73.mCAR-pNK), similarly resulted in efficient killing of SJ-GBM2 (pediatric), GBM43 (adult) and GBM10 (recurrent) cells, to significantly higher levels than those of control non-engineered NK cells (see FIG. 6 and FIG. 7, subparts F-H). Live imaging of the killing of GBM targets by native or engineered NK cells demonstrates the dynamic nature of this process and a higher killing specificity of GBM cells by CD73.mCAR-pNK cells as compared to that by native human NK cells (see FIG. 9: GBM cells were seeded into a 24-well plate at a density of 4×10⁴ cells/well. After overnight culture, either pNK or CD73.m.CAR-pNK cells were added to an E/T ratio at 5. The co-cultures were then imaged using an IncuCyte S3 with scans performed every 10 min for 4 hours).

Stimulation by GBM cells contributed to a significantly increased NK cell degranulation and intracellular production of IFN-γ by CD73.mCAR-pNK cells (FIG. 7, subparts I and J). CD73.mCAR-NK cells lacking the anti-CD73 scFv following uPA treatment displayed significantly decreased cytotoxic ability of target GBM43 cells after co-culture at effector/target (E/T) ratios of 2.5 and 5 for 4 h (see FIG. 7, subpart K). In addition, after treatment with anti-CD73 scFv cleaved from CD73.mCAR-pNK cells, GBM43 cells showed a significantly reduced ability to produce adenosine due to the loss of active CD73 (FIG. 7, subpart L).

Accordingly, the anti-tumor specificity imparted upon NK cells by genetic expression of the multifunctional construct resulted in enhanced resistance to the loss of CD16 upon contact with GBM cells (FIG. 7, subpart M), as well as a reduced upregulation of NKG2A (FIG. 7, subpart N) as compared to changes observed on non-engineered human NK cells in response to GBM.

Finally, to address any potential off-target effects due to multi-specific targeting of any potential expression of these antigens on non-tumor tissues, the ability of CD73.mCAR-pNK cells to target healthy cells was also evaluated using the protocols set forth herein.

CD73.mCAR-pNK cells did not preferentially kill normal cells, specifically those belonging to neural lineages including hCMEC/D3 and HCN-2 cells. Instead, CD73.mCAR-pNK cells exhibited effector activity comparable to that of control non-engineered pNK cells against healthy brain cells (see FIG. 7, subparts O and P).

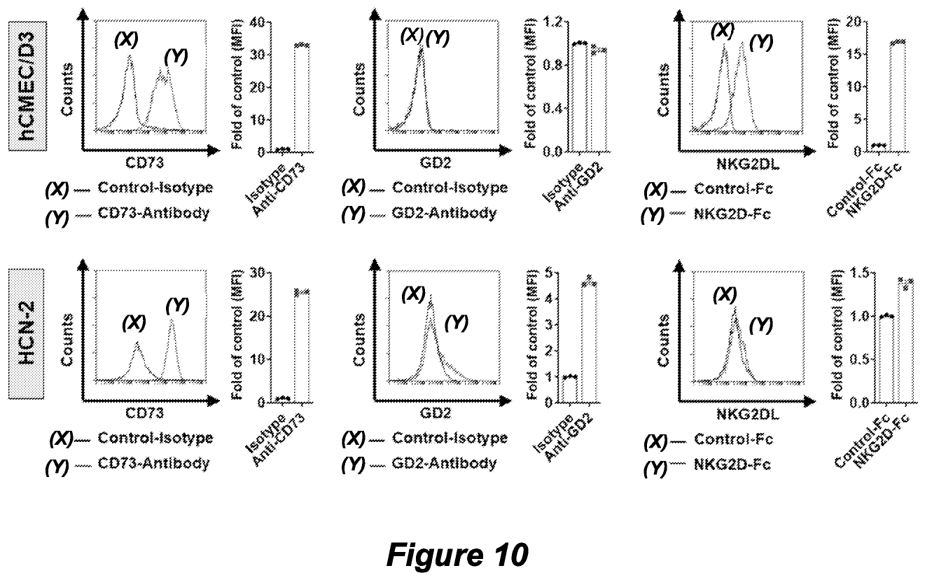

Although multiple mechanisms may be involved in the targeting of such cells, including for example, a distinct pattern of inhibitory ligand expression, the low cytotoxicity rates induced by CD73.mCAR-pNK cells could be due to the relatively lower expression profiles for the targeted ligands on these cells compared with GBM cells (see FIG. 10). Together, these observations indicate the functional superiority of CD73.mCAR-pNK cells, and represent a promising path forward for highly specific anti-GBM immunotherapies without observable toxicities to normal cells.

### Example 3

Functional Targeting of Autophagy in GBM Enhances NK Activity and Honing Autophagy is a critical cell survival mechanism leading to and driving cancer development and progression. To tamper with its ability to drive GBM resistance to therapy and establish sensitization of GBM to immune cell treatment, the effects of blocking autophagy in GBM were investigated via two approaches. In one study, autophagy was blocked genetically, through the targeting the BECN1 gene via the generation of knockdown patient-derived GBM cells (BECN1⁻GBM43). More specifically, GBM43 cells were grown as previously described. Lentiviral BECN1 shRNA particles were used, according to the manufacturer's instructions, to generate BECN1⁻ GBM43 cells. In vitro growth behavior of BECN1⁻ GBM43 was verified via CCK8 assay analysis.

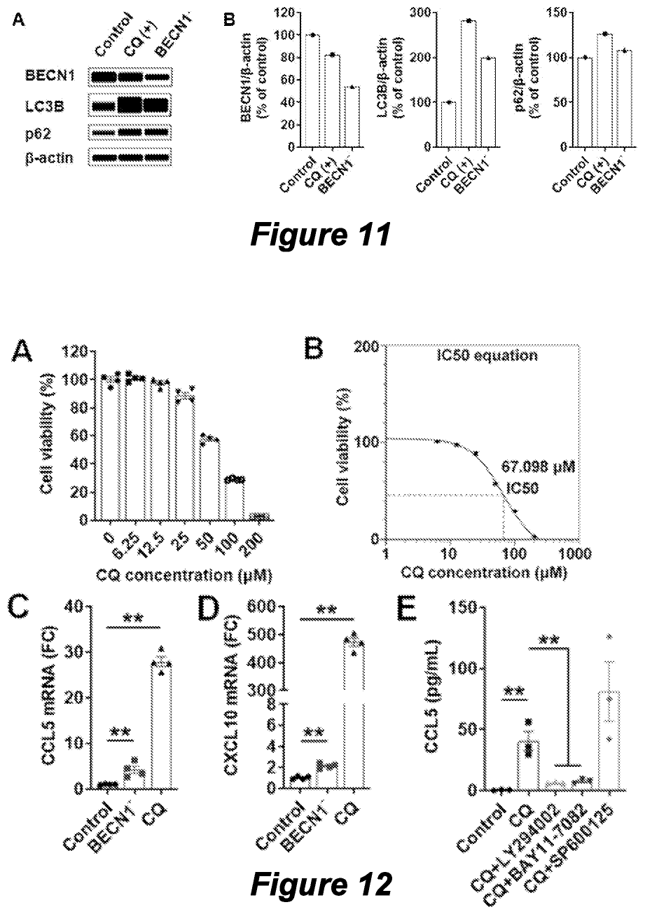

Alternatively, autophagy was inhibited pharmacologically via treatment with chloroquine (CQ), a common FDA-approved autophagy inhibitor (see FIG. 11). These targeting approaches were tested on both NK cell infiltration and effector functions. For this approach, GBM43 cells were treated with different concentrations of CQ. To determine in vitro cell viability in response to CQ, GBM43 cells were seeded in 96-well plates at a density of 1×10⁴ cells/well. After overnight culture, the cells were treated with different concentrations of CQ and incubated for another 24 hours. The cell viability was then determined with CCK-8 assay analysis.

Cell lysates were prepared using RIPA lysis buffer system according to the manufacturer's instructions. After that, the samples were run and analyzed via auto-western service provided from RayBiotech, Inc. The target autophagic markers include BECN1, LC3B, p62 and R-actin (with Beclin 1 (BECN1), LC3-II (LC3B) and p62 being the three primary markers of autophagy).

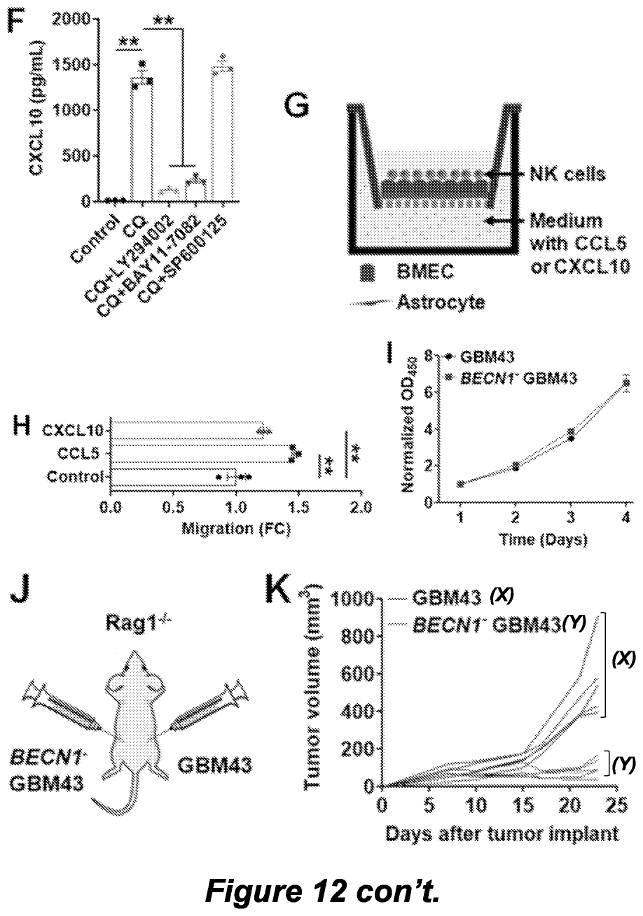

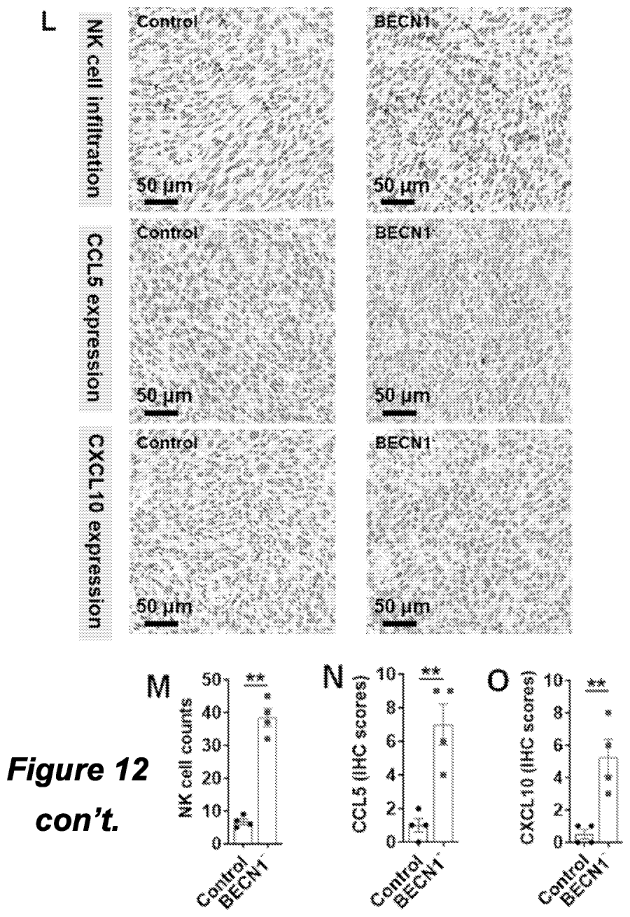

Significantly, in all cases, the administration of CQ impaired viability and proliferation of GBM43 cells in a dose-dependent manner after 24 h (IC₅₀=67.10 μM; see FIG. 12, subparts A and B).

The gradient between chemokines and their cognate receptors initiates the directional movement of cells to sites with higher concentrations to ultimately regulate immune cell trafficking to tumors. Stimulated by a paucity of insight into chemokine-mediated trafficking of NK cells to GBM, the consequence of inhibiting autophagy in GBM on the expression levels of a number of chemokines known to be associated with NK cell trafficking to tumors was studied.

### RNA Extraction and RT-PCR

Total RNAs were extracted using the mirVana™ miRNA Isolation Kit and the concentrations determined with a Qubit 4 Fluorometer. The RNA (80 ng) from each sample was reverse-transcribed using the qScript™ One-Step SYBR® Green qRT-PCR kit in a ViiA-7 RT-PCR system (Thermo Fisher Scientific, Waltham, Mass.). The GAPDH gene was used as the endogenous control. The comparative Ct values of genes of interest were normalized to the Ct value of GAPDH. The 2^{-Δct} method was used to determine the relative expression of the genes, while the 2^{-ΔΔct} method was used to calculate fold changes of gene expression over control.

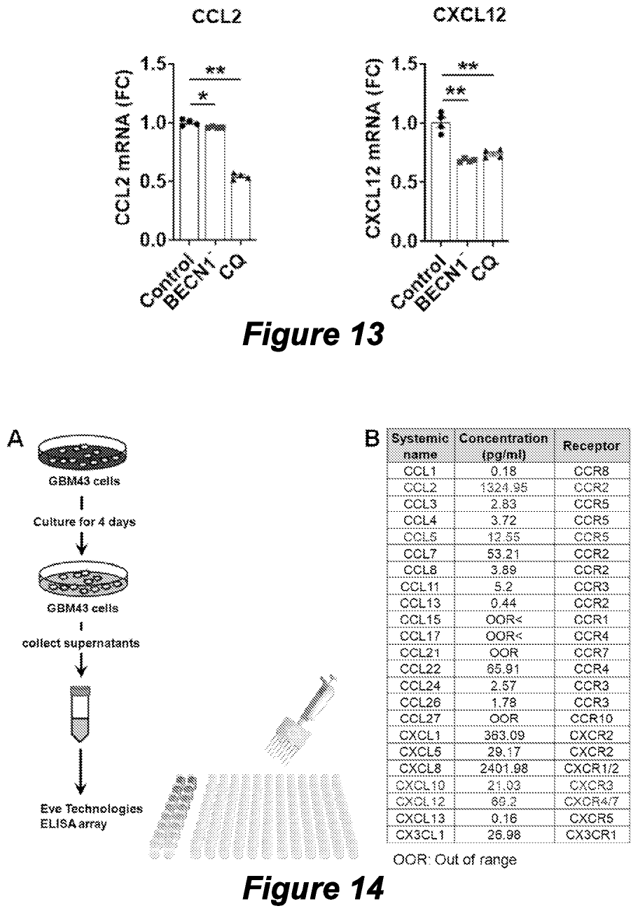

Both BECN1⁻GBM43 and CQ-treated GBM43 cells resulted in a substantial upregulation of CCL5 and CXCL10 at the transcriptional level as measured by a significant increase in their mRNA (FIG. 12, subparts C and D). At the same time, the chemokines CCL2 and CXCL12 showed decreased transcriptional levels on GBM43 cells upon inhibition of autophagy (FIG. 13).

### ELISA Measurements

To evaluate changes in the protein levels of these chemokines and gain more insight into the underlying molecular mechanisms, chemokine concentrations were quantified in the conditioned media of CQ-treated GBM43 cells using ELISA in absence and presence of various pharmacological inhibitors driving signaling pathways for these chemokines. This included the PI3K inhibitor LY294002, NF-κB inhibitor BAY11-7082 and JNK inhibitor SP600125.

More specifically, to measure secreted CCL5 and CXCL10, GBM43 cells were treated with CQ at a final concentration of 50 μM in presence or absence of various small molecule inhibitors, including LY294002, BAY11-7082 and SP600125 for 24 hours. Supernatants were collected and the levels of CCL5 and CXCL10 were quantified using Human CCL5 and CXCL10 Biolegend-ELISA MAX™ Deluxe Sets according to the manufacturer's directions. To look at the levels of multiple chemokines secretion from GBM43 cells, the cell culture supernatants were also collected and all the selected targets were determined with the chemokine array provided by Eve Technologies (Calgary, Canada).

The conditioned medium of control GBM43 cells displayed relatively low levels of both CCL5 (12.55 μg/mL) and CXCL10 (21.03 μg/mL) as compared with their expression of CCL2 (1324.95 μg/mL) and CXCL12 (69.2 μg/mL) (see FIG. 14 and, in particular, subpart B thereof). However, CQ-treated GBM43 cells secreted significantly higher amounts of both CCL5 and CXCL10 as compared with untreated control cells across the board (see FIG. 12, subparts 4E and 4F). The elevated production of these chemokines was significantly inhibited by LY294002 or BAY11-7082, but not by SP600125, which suggests a mechanistic link between enhanced expression of CCL5 and CXCL10 upon inhibition of autophagy on GBM and activation of the PI3K/NF-κB pathway.

### Transwell BBB Migration Assay

To determine involvement of CCL5 and CXCL10 in the migration of NK cells, the migration of activated pNK cells toward CCL5 and CXCL10 gradients was then tracked using an in vitro BBB model setup by a direct contact co-culture of primary human astrocytes and hMEC/C3 human cerebral microvessel endothelial cells (FIG. 12, subpart G).

An in vitro direct contact co-culture blood-brain barrier (BBB) model was established as previously described⁵³. Briefly, human astrocytes were seeded at a density of 4×10⁴ cells/cm² into 24-well Transwell® inserts (pore size 5 μm) pre-coated with 2 μg/cm² poly-L-lysine and allowed to proliferate/differentiate for 48 hours in media. Media was then removed and the hCMEC/D3 cells suspended into EBM-2 media were seeded at a density of 1×10⁵ cells/cm².

The co-culture was grown in EBM-2 with media changes every other day for an additional 7 days before studies were conducted. 600 μL of RPMI1640 medium supplemented with 1% FBS containing recombinant CCL5 or CXCL10 at 100 ng/mL was placed in the lower chamber of the Transwell® plate. Activated pNK cells (5×10⁵) in 100 μL RPM11640 medium supplemented with 1% FBS were placed into the upper chamber (5-μm pore size). After incubation for 6 hours at 37° C. and 5% CO₂, the number of pNK cells that migrated into the lower chamber was determined by flow cytometry. Data are presented as percentage of migration based on total cell input.

As shown in FIG. 12, subpart H, activated pNK cells significantly increased in their ability to migrate along CCL5 (100 ng/mL) as well as CXCL10 (100 ng/mL) ligand gradients. Interestingly, the viability and proliferation of BECN1⁻GBM43 cells were unaffected by BECN1 knockdown (FIG. 12, subpart I)

### In Vivo Studies

The impact of BECN1 knockdown on GBM tumor growth and NK cell infiltration was further evaluated in vivo in Rag1^{-/-} mice, which bare no mature B and T lymphocytes but instead possess a mature NK cell compartment (FIG. 12, subpart J).

GBM43 cells (3×10⁶) were inoculated subcutaneously (SC) in both flanks (GBM43 control cells in the right flanks and BECN1⁻GBM43 cells in the left flanks) of Rag1^{-/-} mice. The tumor growth was monitored and the length (L), width (W) and height (H) of the tumor were measured using a digital caliper. The tumor volume (mm³) was calculated using the formula: V=0.52×L×W×H. Tumor growth was monitored until mice met predefined endpoint criteria. At that point, the tumor tissues were harvested and processed for histologic (IHC) analyses.

Subcutaneous GBM xenografts were established by inoculating NSG mice with 3×10⁶ GBM43 cells in the right flank. Ten days later, when tumors reached a volume of about 80 mm³, mice were randomly assigned to 5 groups (n=4/group) and treated according to one of the following protocols: (1) PBS only; (2) intravenous (IV) injection of 5×10⁶ pNK cells alone; (3) intravenous (IV) injection of 5×10⁶ CD73.mCAR-pNK cells alone (Construct 1); (4) intraperitoneal (IP) injection of CQ (50 mg/kg) alone. NK cells were administered once a week for three weeks. The mice receiving adoptive NK cells therapy (groups 2 and 3) also received IL-15 (0.5 μg/mice) by IP injection every 3-4 days. Tumor growth was followed by caliper measurements and tumor volumes were calculated using the formula: V=0.52×L×W×H as described above. Body weights of the mice were recorded throughout the treatment. At the end of the therapy, the mice were sacrificed and tumors were harvested for histologic (IHC/IF) analyses.

Additionally, orthotopic GBM xenografts were generated using NSG mice as previously described. Briefly, on day 0, the GBM43 (Luc) cells (1×10⁵) were stereotactically implanted into the right forebrain through a digitalized stereotactic delivery system. 9 days following implantation, mice were randomly assigned to 4 groups (n=4/group) and treated according to one of the following protocols: (1) untreated (as the control); (2) intraperitoneal (IP) injection of CQ (50 mg/kg) alone; (3) intracranial (IC) injection of 2×10⁶ CD73.mCAR-pNK cells (expressing Construct 1) alone; (4) combination of CQ (IP) and CD73.mCAR-pNK cells (IC). CQ was continually injected 3 times a week (once/day) before administration of CD73.mCAR-pNK cells. CD73.mCAR-pNK cells were administered once a week for three weeks. Tumor volumes were monitored and recorded using the Spectral Ami Optical imaging system. Body weights of the mice were also recorded during the treatment period. At the end of the treatment, the mice were sacrificed and whole brain tissues from each mouse were harvested for adenosine measurement and IHC/IF analyses.

Immunohistochemistry (IHC) and immunofluorescence (IF) staining were carried out at the Histology Research Laboratory at the Purdue University College of Veterinary Medicine (West Lafayette, Ind.). Briefly, the tumors were fixed in 10% neutral-buffered formalin, embedded in paraffin, and cut into 3-5 μm sections.

For BECN1⁻ GBM43 xenografts, the mouse NK cells in tumors were detected through the staining using mouse NKp46/NCR1 antibody. For the quantification of NK cells in the tumors, the stained cells were counted in 5 randomly selected intratumoral fields of each slide at 200× magnification.

CCL5 and CXCL10 expression in the tumors were detected through the staining using human CCL5/RANTES antibody and CXCL10 antibody respectively. The staining was evaluated based on the intensity (weak=1, moderate=2, and high=3) of chemokine immunostaining and the density (0%=0, 1-40%=1, 41-75%=2, >76%=3) of positive tumor cells. The final score of each sample was multiplied by the intensity and density.

For subcutaneous GBM43 xenografts, NK cell infiltration was investigated by IF staining performed with the following stains: Protein L (Alexa 647, far red), NKp46 (Alexa 488, green) and DAPI nuclear counterstain. CCL5 and CXCL10 expression were evaluated by IHC staining as described above. CD73 expression in the tumors was detected through the staining using human CD73 antibody.

For intracranial GBM xenografts, the NK cells in tumors were detected through the staining using human NKp46/NCR1 antibody. CCL5 and CXCL10 expression in the tumors were detected through the staining using human CCL5/RANTES antibody and CXCL10 antibody, respectively. CD73 expression in the tumors was detected through the staining using human CD73 antibody.

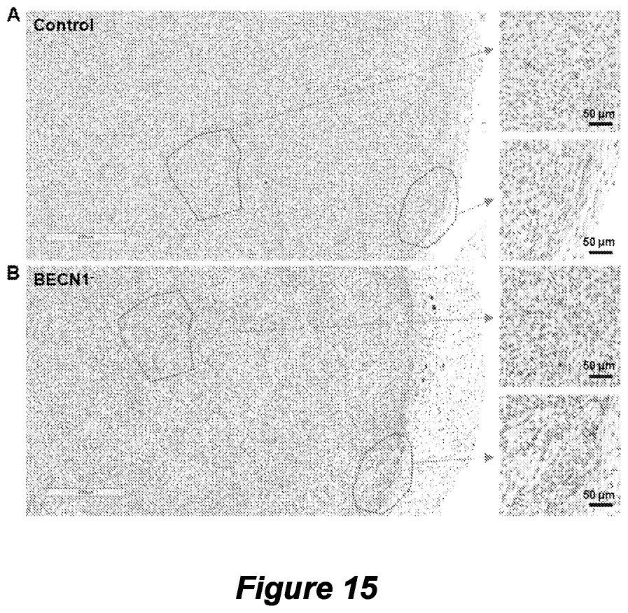

Tumors from the left flanks of mice engrafted with BECN1⁻GBM43 cells exhibited significantly slower growth and a smaller size than those arising from control GBM43 cells (FIG. 12, subpart K). Immunohistochemical staining was performed on extracted tumors to detect the expression of NKp46 as a measure of murine NK cell presence, alongside staining for CCL5 and CXCL10. A significantly higher expression of NKp46 was detected in tumors lacking BECN1, demonstrating a deeper infiltration of NK cells into GBM lacking the ability to perform autophagy compared with control tumors (FIG. 12, subparts L and M). In addition, NK cells in BECN1⁻ tumors showed a higher distribution both at the tumor periphery and in intratumoral areas (see FIG. 15). Further, the expression of CCL5 and CXCL10 was upregulated from relatively low baseline levels in control tumors to significantly higher levels in BECN1⁻ tumors (FIG. 12, subparts L, N, and O).

In addition to the increase in NK cell intratumoral infiltration, NK2G2DL expression (MFI and percentage) on GBM43 cells after 24 hours of treatment with various concentrations of CQ was evaluated.

GBM43 cells were first treated with CQ (varying concentrations), then collected and re-seeded into 96 wells. After overnight culture, the NK cells (NK92 control cells and pNK cells against BECN1⁻GBM43) were added at an E/T ratio of 5 and co-incubated for 4 h.

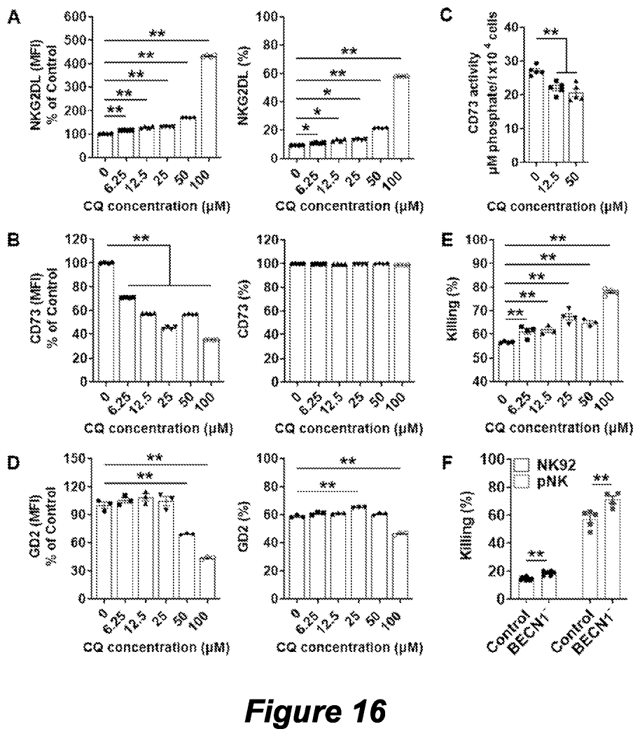

The resulting data supports that CQ-mediated inhibition of autophagy has a measurable and significant effect on the expression of NKG2DL, CD73 and GD2 on GBM43 cells. Specifically, NKG2DL expression (both % and MFI) on GBM43 cells increased significantly after treatment with CQ at various concentrations for 24 h (FIG. 16, subpart A). On the other hand, expression of CD73 (MFI) decreased following CQ treatment, even at a low concentration of 6.25 μM (˜70% of control) (FIG. 16, subpart B).

In addition to the foregoing, intratumoral adenosine concentrations were also assessed. Brain tissues were harvested post-treatment, rinsed with cold PBS and homogenized in PBS. After that, the suspension was centrifuged at 10,000 g for 10 minutes at 4° C. and the supernatant was collected. Adenosine concentrations were determined using an Adenosine Assay Kit according to the manufacturer's directions. The IHC staining was performed as described above.

Production of adenosine in human GBM cells mediated by CD73 was also significantly reduced after treatment with CQ (FIG. 16, subpart C). Expression of GD2 (MFI) decreased after treatment with CQ at concentrations of 50 μM or higher one. Conversely, the percentage expression (%) of GD2 decreased in response to up to 100 μM CQ, after which it remained constant (˜46%) compared to that of control cells (˜58%) (FIG. 16, subpart D).

To further investigate the impact of the inhibition of autophagy on the cytotoxic capacity of NK cells, CQ-treated GBM43 cells or BECN1⁻ GBM43 cells were incubated with pNK cells for 4 h at an E/T ratio of 5. The results support that treatment with CQ did in fact sensitize GBM cells to superior killing by human NK cells (see FIG. 16, subparts E and F).

Taken together, the data resulting from these studies demonstrates that that targeting autophagy improves the infiltration of NK cells into the GBM tumor bed and is aided by higher levels of the chemokines CCL5 and CXCL10. In addition, disabling autophagy can also potentiate NK cell-mediated cytotoxicity against GBM cells by reprogramming their phenotypic signatures in favor of enhanced NK cell functionality, such as increased NKG2DL expression. The complex and unique reprogramming of the GBM TME induced by targeting autophagy can further sensitize treatment of a GBM tumor with NK cells engineered to exploit these reprogrammed pathways.

### Example 4

### Multifunctional Engineered NK Cells Efficiently Target Patient-Derived GBM Tumors In Vivo

To evaluate the in vivo antitumor activity of multifunctional engineered NK cells as well as the effects induced by targeting autophagy on GBM, a subcutaneous xenograft model was established by engrafting patient-derived GBM43 cells into NSG mice.

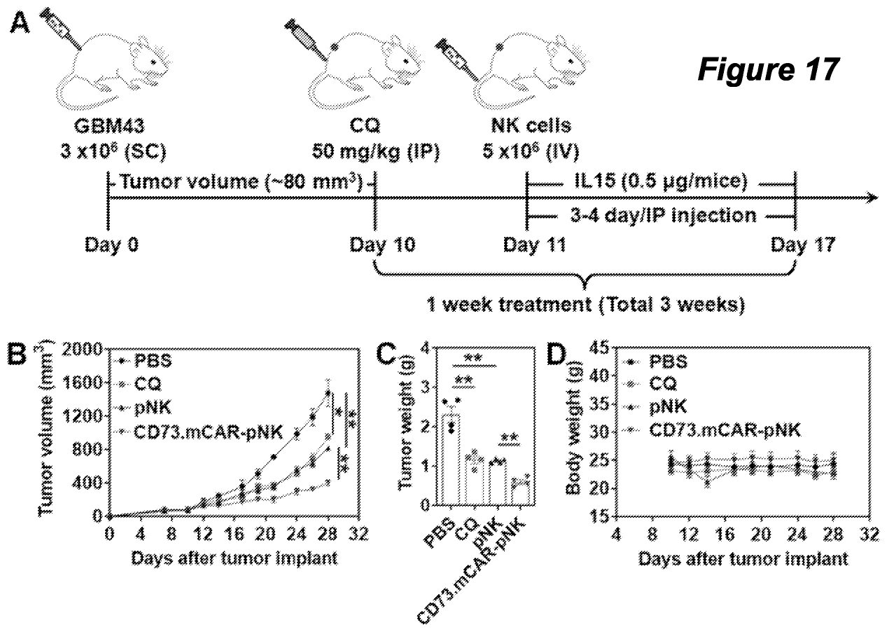

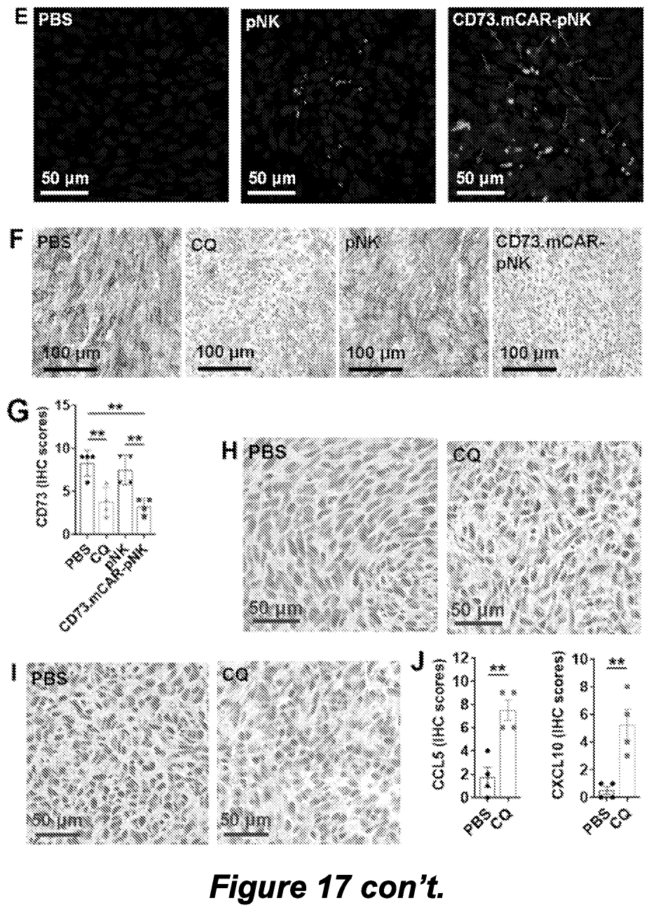

The treatment schedule is summarized in FIG. 17, subpart A. Briefly, 3×10⁶ GBM43 cells were subcutaneously (SC) implanted into the right flank of the mice (Day 0). 10 days later (Day 10), mice in the CQ group were intraperitoneally (IP) injected with CQ at 50 mg/kg for 3 weeks, once a week. One day later (Day 11), the mice in pNK and CD73.mCAR-pNK groups were treated with 5×10⁶ adoptively-transferred pNK or CD73.mCAR-pNK cells intravenously (IV), once a week for 3 weeks. Starting on the day of the first injection of NK cells, all mice received 0.5 μg of IL-15 once every 3-4 days IP.

Treatment with pNK cells significantly delayed tumor growth as compared to mice in the PBS control group (mean tumor volume: 815.75 mm³ vs 1475 mm³, **p<0.01; mean tumor weight: 1.13 g vs 2.31 g, **p<0.01; FIG. 17, subparts B and C). Between the pNK cell-treated mice and the CD73.mCAR-pNK cell-treated mice, the CD73.mCAR-pNK cells mediated more potent antitumor responses and exhibited significantly reduction in the tumor growth rate (mean tumor volume: 401.5 mm³ vs 815.75 mm³, **p<0.01; mean tumor weight: 0.59 g vs 1.13 g, **p<0.01). CQ treatment also showed significant inhibition of tumor growth as compared with the PBS group (mean tumor volume: 953.25 mm³ vs 1475 mm³, *p<0.05; mean tumor weight: 1.16 g vs 2.31 g, **p<0.01; FIG. 17, subparts B and C). There was no significant decrease in body weight of the mice in all groups throughout the entire treatment period (FIG. 17, subpart D).

NK cell infiltration into GBM xenograft tumors was then investigated by IF staining. As shown in FIG. 17, subpart E, adoptively transferred NK cells (light dots) were observed in tumor tissues from pNK cell-treated mice. In comparison, higher NK cell numbers were detected in tumors from CD73.mCAR-pNK cell-treated mice.

Additionally, the locally-cleaved anti-CD73 scFv (indicated by arrows and darker dots) that are able to bind CD73⁺ tumor cells in vivo were detected in CD73.mCAR-pNK cell-treated mice in the vicinity of tumor cells. In vitro data reported here and in previous in vivo studies demonstrated the need for intratumoral protease activity to trigger ligand cleavage. For that reason, its activation in blood was not expected and, as a result, there should not be a detectable presence of scFv in the circulation. Therefore, IF was used to detect tumor-specific presence of CD73 scFv.

The presence of CD73 scFv co-localized only with CD73.mCAR-pNK staining, indicating the CD73 scFv was successfully cleaved. Moreover, a significant decrease in expression of CD73 on tumors in the CD73.mCAR-pNK treatment group was observed as compared to other treatment groups (FIG. 17, subparts F and G). Additionally, tumor tissues from CQ-treated mice revealed a significantly higher presence of the chemokines CCL5 and CXCL10 (FIG. 17, subparts H-J). These tumors also displayed a lower level of CD73 expression (FIG. 17, subparts F and G).

### Example 5

### Activity of the Combination of Multifunctional Genetically-Engineered NK Cells with CQ Against Orthotopic Patient-Derived GBM Xenografts

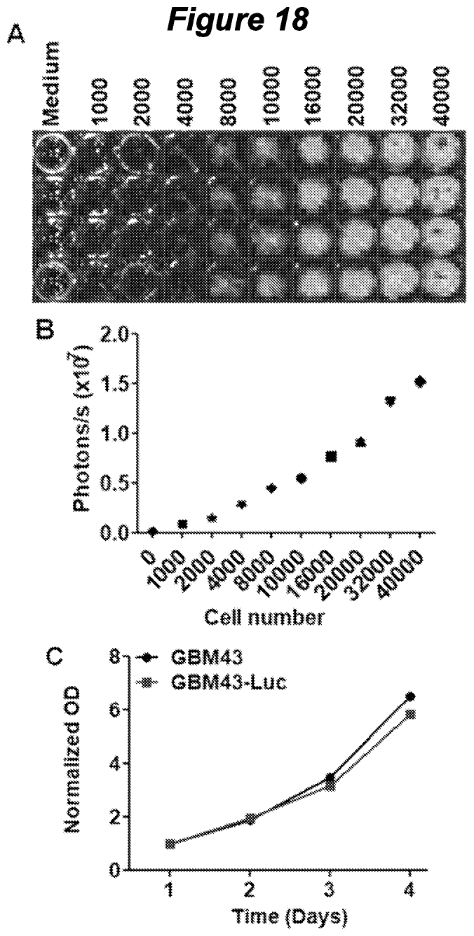

To further analyze the synergistic effect achieved by combination of CQ with CD73.mCAR-pNK cells (expressing Construct 1), a GBM43 xenograft orthotopic model in NSG mice was established. First, GBM43 cells were genetically manipulated to express firefly luciferase (GBM43-Luc) to enable the monitoring of tumor growth via in vivo bioluminescence imaging (see FIG. 18). More specifically, cells (1000) were grown for 4 days in regular growth medium without puromycin. Total viability of cells over time was measured using the CCK-8 assay and plotted in a logarithmic scale. Both cells showed similar growth patterns and doubling times.

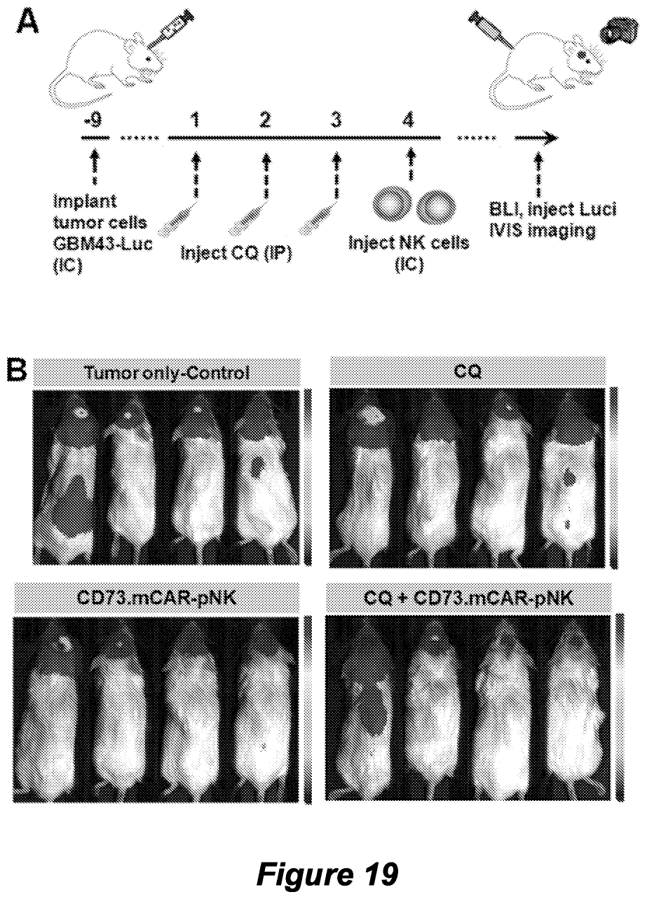

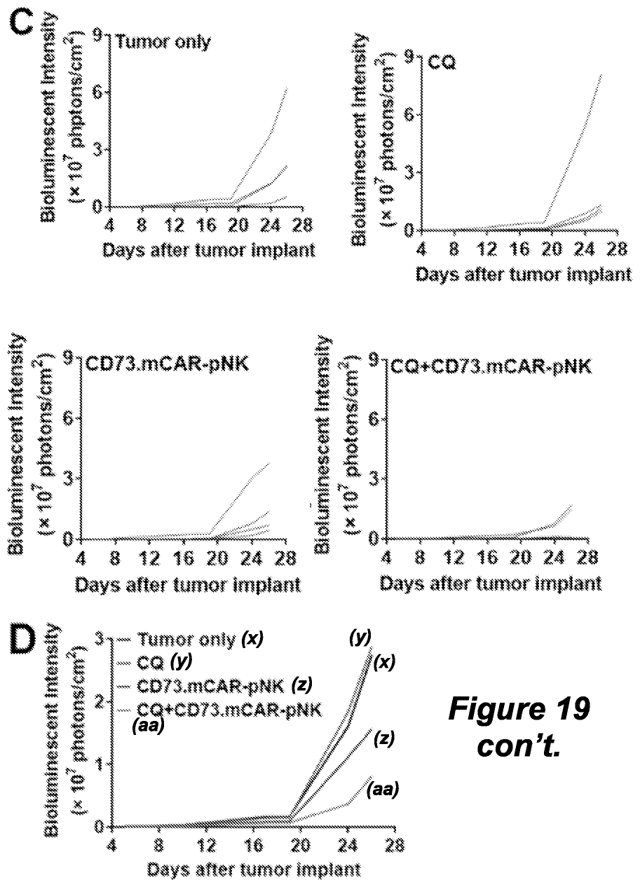

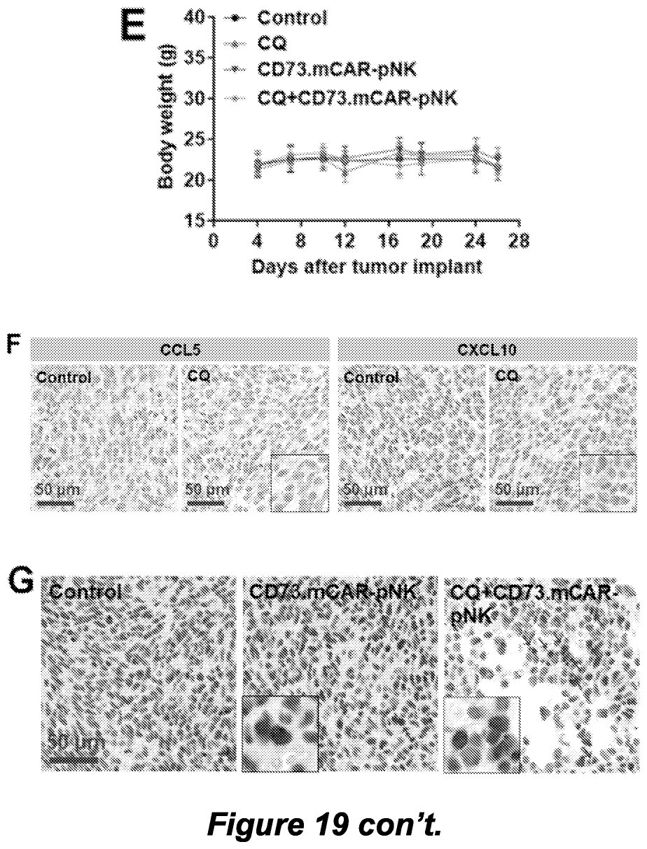

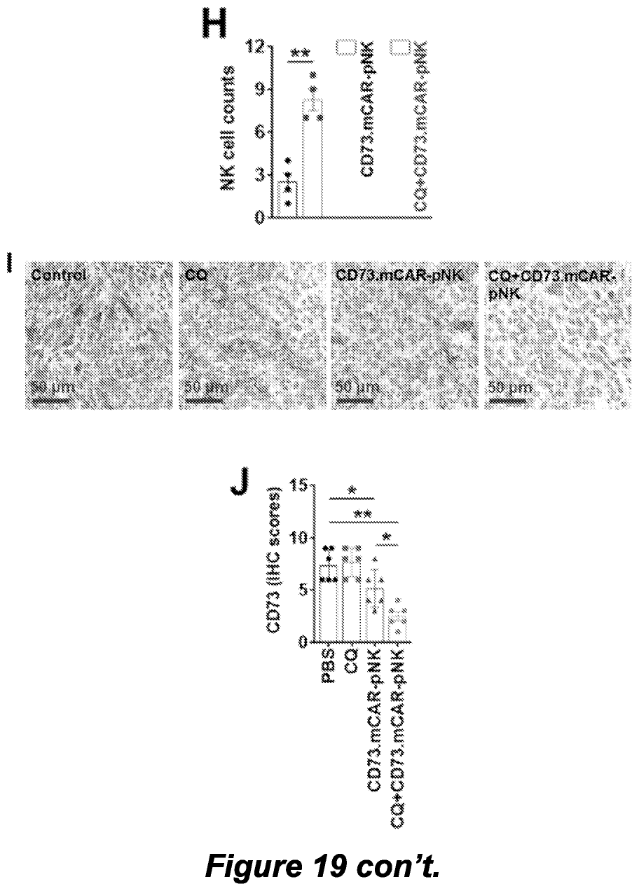

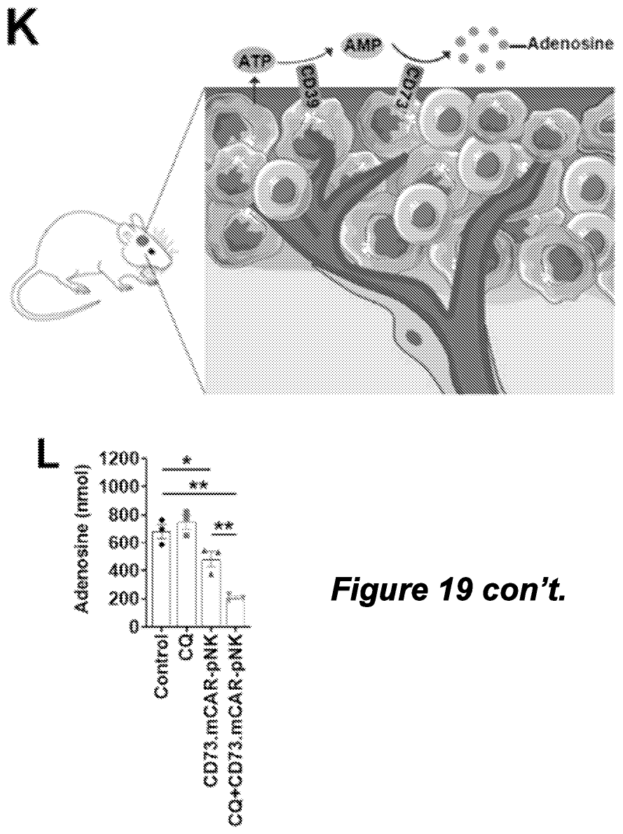

A schematic diagram illustrating the in vivo treatment program used herein is shown in FIG. 19, subpart A. Briefly, NSG mice were orthotopically implanted with GBM43-Luc cells (˜8×10⁴) and treated on day 10 post-implantation with weekly intraperitoneal (IP) injections of CQ for three weeks (50 mg/kg per day, 3 continuous days) and/or intracranial (IC) injections of CD73.mCAR-pNK cells (2×10⁴). When compared with those in the control group, mice treated with CQ alone showed no significant effect on tumor growth (see FIG. 19, subparts B-D).

Conversely, mice that were treated with either CD73.mCAR-pNK cells or CQ+CD73.mCAR-pNK cells showed obviously reduced tumor growth as determined by bioluminescence imaging (see FIG. 19, subpart B). The most potent anti-tumor response was seen with CQ+CD73.mCAR-pNK cells treated-mice. In particular, tumors on half of the mice treated with CQ+CD73.mCAR-pNK cells showed significant arrest during the treatment period. No significant change in body weight of the mice in all groups were found throughout the entire treatment period (FIG. 19, subpart E).

Similar to subcutaneous xenograft studies, tumor tissues of mice in CQ-treated groups displayed robustly up-regulated chemokine expression, including CCL5 and CXCL10 (FIG. 19, subpart F), which may contribute to the increased NK cell infiltration and lead to improved therapeutic efficacy. As shown in FIG. 19, subparts G and H, a significantly higher NK cell infiltration was found in GBM tumors of CQ+CD73.mCAR-pNK cell-treated mice as compared to mice that received CD73.mCAR-pNK cells alone. Compared to control or CQ-treated groups, the tumors of mice in both CD73.mCAR-pNK cells- and CQ+CD73.mCAR-pNK cells-treated groups displayed decreased CD73 expression, with the latter group showing the most substantial loss of CD73 expression (FIG. 19, subparts I and J). This was associated with a significantly decreased level of extracellular adenosine detected in the tumors of treated mice. Specifically, CD73-mediated adenosine production in local brain tissues was decreased most prominently in mice treated with CQ+CD73.mCAR-pNK cells (FIG. 19, subparts K and L).

When taken together, these in vivo xenograft studies demonstrate potent and specific activity of CD73.mCAR-pNK cells against patient-derived adult GBM. The responses were associated with the induction of significant changes in chemokine secretion and CD73 expression on GBM following co-therapy with CQ, indicating a reorganization of the GBM TME induced upon disabling autophagy.

1. A polynucleotide construct comprising
a first sequence encoding at least a first binding domain or fragment thereof comprising a natural killer (NK) activating receptor or a first protein specific for a first cancer-associated antigen, and
a second sequence encoding at least a second binding domain or fragment thereof and a cleavable linker, the second binding domain specific for an adenosine producing cell surface protein of the target cell or an adenosine-intermediary producing cell surface protein of the target cell and the cleavable linker operably linked to the first binding domain.

2. The polynucleotide construct of claim 1 comprising a third sequence encoding at least a third binding domain or a fragment thereof comprising a NK activating receptor or a second protein specific for a second cancer-associated antigen.

3. The polynucleotide construct of claim 2, wherein one of the first and third binding domains or fragments thereof comprises a first protein specific for a cancer-associated antigen and the other of the first and third binding domains or fragments thereof comprises an NK activating receptor.

4. The polynucleotide construct according to claim 1, wherein the second binding domain comprises an antibody fragment specific for CD73, CD39, or CD38.

5. (canceled)

6. The polynucleotide construct of claim 1, wherein the first sequence encodes an NK activating receptor comprising an extracellular ligand-binding domain comprising a natural killer group 2 member D receptor (NKG2D), NKp30, NKp46, NKp40, or DNAM-1.

7. The polynucleotide construct of claim 2, wherein the first cancer-associated antigen and/or the second cancer-associated antigen is/are selected from a group consisting of disialoganglioside (GD2), ganglioside G3 (GD3), Her 2 (p185), CD19, CD20, CD56, CD123, CD22, CD30, CD33, CD171, CS-1, C-type lectin-like molecule-1, EpCAM, G250, proteoglycans, GD2, MHC II, TAG-72, milk mucin core protein, Lewis A antigen, tyrosine-protein kinase transmembrane receptor (ROR1), c-met, epidermal growth factor receptor (EGFR), EGFR variant III, and carcinoembryonic antigen (CEA).

8. (canceled)

9. The polynucleotide construct of claim 1, wherein the first binding domain or fragment thereof comprises an extracellular ligand-binding domain comprising a natural killer group 2 member D receptor (NKG2D) and the first sequence further encodes upregulated expression of the NKG2D as compared to expression of NKG2D in a wild-type NK cell.

10. (canceled)

11. The polynucleotide construct of claim 2, wherein the first sequence is expressed in a first chimeric antigen receptor (CAR), and the second sequence is expressed in a second CAR.

12. The polynucleotide construct of claim 2, wherein one or both of the first and third sequences encode one or more signaling domains for promoting cytotoxic or cytolytic activity upon activation.

13-15. (canceled)

16. The polynucleotide construct according to claim 12, wherein one or more signaling domains are selected from a group consisting of an immunoglobulin γ-Fc region receptor III-A (FcγRIIIA), a cluster of differentiation 28 (CD28), a tumor necrosis factor receptor superfamily member 9 (TNFRSF9 or 4-1BB), a tumor necrosis factor receptor superfamily member 4 (TNFRSF4 or OX40), a Fas ligand (FasL), a TNF-related apoptosis-inducing ligand (TRAIL), DNAX-activating protein 10 (DAP10), DNAX-activating protein 12 (DAP12), natural cytotoxicity receptor NKp46, natural cytotoxicity receptor NKp44, natural cytotoxicity receptor NKp30, lymphocyte function-associated antigen 1 (LFA-1), cluster of differentiation 244 (CD244), CD137, CD3 zeta (CD3ζ) and a NKG2D-DAP10 receptor complex.

17-18. (canceled)

19. The polynucleotide construct of claim 1, wherein the first sequence further encodes a hinge domain, one or more self-cleaving peptides, or both.

20-21. (canceled)

22. The polynucleotide construct of claim 17, wherein the first sequence encodes a first amino acid that has at least a 90% sequence identity to SEQ ID NO: 7 and the third sequence encodes a second amino acid that has at least 90% sequence identity to SEQ ID NO: 6.

23. An engineered cell or cell line that expresses a polynucleotide construct according to claim 1.

24. The engineered cell or cell line of claim 23 that expresses an amino acid sequence that has at least a 90% sequence identity to SEQ ID NO: 8.

25-27. (canceled)

28. A method of treating a subject suffering from a cancer using an immunotherapy treatment, the method comprising administering, or having administered, to a subject a therapeutically effective amount of a pharmaceutical composition comprising a population of engineered natural killer (NK) cells expressing a polynucleotide construct encoding at least:
a first binding domain or fragment thereof comprising an NK activating receptor or a first protein specific for a first cancer-associated antigen; and
a second binding domain or fragment thereof and a cleavable linker, the second binding domain specific for an adenosine-producing or adenosine-intermediary-producing cell surface protein of a target cell and the cleavable linker operably linked to the first binding domain.

29. The method of claim 28, wherein the polynucleotide construct further encodes a third binding domain or fragment thereof comprising a NK activating receptor or a second protein specific for a second cancer-associated antigen.

30. The method of claim 29, wherein one of the first and third binding domains or fragments thereof comprises a first protein specific for a cancer-associated antigen and the other of the first and third binding domains or fragments thereof comprises an NK activating receptor, and
the polynucleotide construct further encodes one or more signaling domains operably linked to the first binding domain to promote cytotoxic or cytolytic activity of the engineered NK cells upon activation.

31. The method according to claim 28, wherein the population of engineered NK cells express an amino acid sequence that has at least a 80%, 85%, or 90% amino acid sequence identity to SEQ ID NO: 8.

32. (canceled)

33. The method of claim 30, wherein the cancer is glioblastoma, the first binding domain or fragment thereof comprises a first protein specific for disialoganglioside (GD2), the third binding domain or fragment thereof comprises an extracellular ligand-binding domain comprising a natural killer group 2 member D receptor (NKG2D), and the third binding domain or fragment thereof is specific for CD73.

34. The method of claim 31, further comprising administering, or having administered, to the subject an additional therapeutic treatment comprising an autophagy inhibitor.

35-44. (canceled)
